(19) United States
(12) Patent Application Publication
(10) Pub. No.: US 2024/0012011 A1
(43) Pub. Date: Jan. 11, 2024

(54) METHODS AND ASSAYS FOR SECRETOME ACTIVITY ANALYSIS

(71) Applicant: Combangio, Inc., Menlo Park, CA (US)

(72) Inventors: Spencer Alford, Newark, CA (US); Peter Baciu, Arlington, MA (US)

(21) Appl. No.: 18/349,107

(22) Filed: Jul. 7, 2023

Related U.S. Application Data
(60) Provisional application No. 63/359,743, filed on Jul. 8, 2022.

(51) Int. Cl. G01N 33/68; A61P 27/02; A61K 38/20; A61K 45/06; A61K 38/18; A61K 38/17; A61K 38/57

(52) U.S. Cl. CPC G01N 33/6893; A61P 27/02; A61K 38/2053; A61K 45/06; A61K 38/1866; A61K 38/1833; A61K 38/1729; A61K 38/57

(57) ABSTRACT

The present application provides methods and assays for assessing a mesenchymal stem cell secretome in order to use the MSC secretome in methods of treating ocular conditions and/disorders.

### CROSS-REFERENCE TO RELATED APPLICATIONS

This application claims priority to U.S. Provisional Application No. 63/359,743, filed Jul. 8, 2022, which is herein incorporated by reference in its entirety.

### BACKGROUND OF THE INVENTION

Regenerative medicine is an area of medicine that is concerned with the replacement or regeneration of human cells, tissues, or organs, in order to restore or establish normal functions. For example, stem cell therapies can be utilized in order to treat, prevent, or cure a variety of diseases and disorders.

Stem cells are cells that have the ability to divide without limit and that, under certain specific conditions, can differentiate into a variety of different cell types. Totipotent stem cells are stem cells that have the potential to generate all of the cells and tissues that make up an embryo. Pluripotent stem cells are stem cells that give rise to cells of the mesoderm, endoderm, and ectoderm. Multipotent stem cells are stem cells that have the ability to differentiate into two or more cell types, whereas unipotent stem cells are stem cells that differentiate into only one cell type. One type of such stem cells are mesenchymal stem cells. See, for example U.S. Patent Application US20190046576.

However, it is difficult to produce and store live stem cell-based therapies on a clinically relevant scale. (See, Trainor et al., Nature Biotechnology 32(1) (2014)). Moreover, the therapeutic potency and regenerative capacity of such therapies is often variable and the cells can die before or during transplantation. (See, Newell, Seminars in Immunopathology 33(2):91 (2011)). Implanted stem cells are also susceptible to host immune system attack and/or rejection, and it is often difficult to assess potency and/or control “dosing”. Thus, there is a need in the art for additional regenerative therapies that can overcome the cost, storage, and manufacturing quality control limitations that are currently associated with cell-based regenerative medicine therapies. In particular in the context of ocular conditions.

Blast and blunt injuries to the eye can cause a series of mechanical disruptions to the ocular contents including commotio retinae, traumatic cataract, disruption of the zonular attachments to the lens, angle recession, iris dialysis, and rupture of the pupillary sphincter and disruption to optic nerve. Treatment of these injuries has been limited to mechanical repair (when possible) of the iris, replacement of the crystalline lens with plastic lens implants, and repair of retinal detachments. There has been no treatment to repair the cellular architecture of the retina or the anterior chamber as a result of injury, or disease, or inherited genetic conditions such as retinitis pigmentosis. Furthermore, traumatic optic neuropathy and optic nerve avulsion are among the six leading types of ocular injury that required specialized ophthalmic care during Operation Iraqi Freedom (Cho and Savitsky, “Ocular Trauma Chapter 7”, in Combat Casualty Care: Lessons learned from Oef and Oif, by Brian Eastbridge and Eric Savitsky, pp. 299-342, Ft. Detrick, Md.: Borden Institute (US) Government Printing Office, 2012), incorporated herein by reference in its entirety. Sixty percent of traumatic head injuries result in neuro-ophthalmic abnormalities (Van Stavern, et al., J Neuro-Ophthamol 21(2):112-117, 2001) (incorporated herein by reference in its entirety) half of which involve the optic nerves or visual pathways. Traumatic injury to neurons results in axonal damage and irreversible neuronal loss resulting in permanent deficits. While a number of potential neuroprotective therapies have been identified in animals, these single agents have generally failed to translate to therapies in human clinical trials (Turner, et al., J Neurosurg 118(5):1072-1085, 2013, incorporated herein by reference in its entirety). Combination therapies that affect several cellular targets are likely needed to prevent neuronal damage or restore neuronal function.

The cornea serves a protective role as the outermost tissue of the eye, however it is highly vulnerable to severe injury and disease. Its lack of blood vessels enables its transparency but also limits its ability to heal. Corneal injury, due to its potential to cause irreversible blindness, requires prompt intervention and aggressive treatment. The critical need for improved ocular surface healing therapies is particularly apparent for chemical burns and in severe corneal diseases, such as ocular manifestations of acute Chronic Graft v. Host Disease (GvHD), Stevens-Johnson Syndrome, Ocular Mucous Membrane Pemphigoid and other conditions giving rise to persistent corneal epithelial defect, which collectively comprise an incidence of over 100,000 cases per year. (See, Dietrich-Ntoukas et al. Cornea. 2012, 31(3):299-310; Stevenson W, et al., Clin Ophthalmol. 2013, 7:2153-2158; White K D, et al., J Allergy Clin Immunol Pract. 2018; 6(1):38-69; Tauber J. (2002) Autoimmune Diseases Affecting the Ocular Surface. In: Ocular Surface Disease Medical and Surgical Management. Springer, New York, NY.; and Wirostko B, et al., Ocul Surf 2015 July; 13(3): 204-21; and Haring, R S., et al., JAMA Ophthalmol. 2016 Oct. 1; 134(10):1119-1124.)

Moreover, topical ophthalmic drug development is impeded by many anatomical constraints including tear turnover and dilution, nasolacrimal drainage, and reflex blinking with often less than 5% of the topically administered dose reaching deeper ocular tissues (Gaudana et al., 2009). In the case of corneal wounds, the initial insult causes rifts in the corneal epithelium thereby enabling the passage of topically applied MSC-S to penetrate the epithelial layers.

Accordingly, there is a large unmet need in the art for ocular therapies that can target the eye and deliver a therapeutic payload to difficult-to-reach sensory tissue which may have degenerated due to inflammation secondary to trauma (such as for example, burns, acute inflammation, age, and/or oxidative stress). The present invention meets this need by providing mesenchymal stem cell secretome compositions for use in such treatments, as well as methods for making such compositions.

### BRIEF SUMMARY OF THE INVENTION

The present invention provides methods and assays for mesenchymal stem cell (MSC)-derived secretome activity analysis.

In some embodiments, the present invention provides herein a method for characterizing a MSC secretome, wherein the method comprises:
(i) subjecting an MSC secretome to one or more characterization assays, wherein characterization assays are selected from the group consisting of physical component characterizations, oxidative stress assays, misfolded protein response assays, ER stress assays, cellular response assays, safety analyses, stability assays, proliferation assays, migration assays, adhesion assays, neovascularization assays, differentiation/scarring assays, inflammation/immune assays, gliosis assay, tissue explant survival and function assays, organoid development or survival/function assays, epithelial barrier integrity assays, retinal degeneration assays, and/or assays of inherited retinal disease including human and animal retinal explants, neural protection/neurotrophic assays, wherein the MSC secretome is optionally preconditioned; and
(ii) determining the results from the one or more assays in (i).

In some embodiments, the present invention provides herein a method determining biopotency and stability of a MSC secretome comprising, wherein the method comprises:
(i) subjecting an MSC secretome to one or more characterization assays, wherein characterization assays are selected from the group consisting of physical component characterizations, oxidative stress assays, misfolded protein response assays, ER stress assays, cellular response assays, safety analyses, stability assays, proliferation assays, migration assays, adhesion assays, neovascularization assays, differentiation/scarring assays, inflammation assays, in vivo response to oxidative stress (for example, retinal ischemia reperfusion), epithelial barrier integrity assays, retinal degeneration assays, and/or assays of inherited retinal disease including human and animal retinal explants, neural protection/neurotrophic assays, wherein the MSC secretome is optionally preconditioned; and
(ii) determining the results from the one or more assays in (i).

In some embodiments, the present invention provides herein a method for determining MSC secretome lot consistency between a plurality of MSC secretome lots, wherein the method comprises:
(i) subjecting an MSC secretome to one or more characterization assays, wherein characterization assays are selected from the group consisting of physical component characterizations, oxidative stress assays, misfolded protein response assays, ER stress assays, cellular response assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays, wherein the MSC secretome is optionally preconditioned; and
(ii) determining the results from the one or more assays in (i).

In some embodiments, the results in (ii) from a physical component characterization identify an anti-angiogenic MSC secretome or a composition made comprising said secretome.

In some embodiments, the results in (ii) from a safety analyses provides for a MSC secretome that exhibits blood compatibility, and low and/or no pyrogens and/or endotoxins.

In some embodiments, the results in (ii) from a stability assay provides for a MSC secretome that exhibits stability at −20° C., 4° C., and/or 20° C. (for example, room temperature) for at least 7 days or for at least 14 days.

In some embodiments, the results in (ii) from a proliferation assay provides for a MSC secretome that induces proliferation.

In some embodiments, the results in (ii) from a migration assay provides for a MSC secretome that induces migration.

In some embodiments, the results in (ii) from a neovascularization assay provides for a MSC secretome that inhibits or does not promote neovascularization.

In some embodiments, the results in (ii) from a differentiation/scarring assay provides for a MSC secretome that inhibits differentiation and/or scarring.

In some embodiments, the results in (ii) from an inflammation assay provides for a MSC secretome that inhibits inflammation or alters immune response.

In some embodiments, the method further comprises:
(iii) identifying a MSC secretome lot based on the results in (ii).

In some embodiments, the method comprises a preconditioning step to the secretome.

In some embodiments, the present invention provides a panel of tests and/or assays for characterizing a MSC secretome, wherein the panel comprises at least two characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays, wherein the secretome is optionally preconditioned.

In some embodiments, the present invention provides a panel of tests and/or assays for determining consistency between MSC secretome lots, wherein the panel comprises one or more characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays, wherein the secretome is optionally preconditioned.

In some embodiments, the physical component characterization identifies a MSC secretome as characterized herein or a composition made of said secretome.

In some embodiments, the results in (ii) from a safety analyses provides for a MSC secretome that exhibits blood compatibility, and low and/or no pyrogens and/or endotoxins.

In some embodiments, the stability assay identifies for a MSC secretome that exhibits stability at −20° C., 4° C., and/or 20° C. (for example, room temperature) for at least 7 days or for at least 14 days.

In some embodiments, the proliferation assay identifies for a MSC secretome that induces proliferation.

In some embodiments, the migration assay identifies a MSC secretome that induces migration.

In some embodiments, the neovascularization assay identifies for a MSC secretome that inhibits or does not promote neovascularization.

In some embodiments, the differentiation/scarring assay identifies a MSC secretome that inhibits differentiation and/or scarring.

In some embodiments, the inflammation assay identifies a MSC secretome that inhibits inflammation or alters immune response.

In some embodiments, physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, and/or epithelial barrier integrity assays, retinal degeneration assays, and/or assays of inherited retinal disease including human and animal retinal explants, neural protection/neurotrophic assays are all performed.

In some embodiments, the tests and/or assays identify a MSC secretome.

In some embodiments, the MSC secretome is an anti-angiogenic MSC secretome and/or an anti-scarring MSC secretome.

In some embodiments, the MSC secretome is an anti-angiogenic MSC secretome and/or or an anti-scarring MSC secretome.

In some embodiments, the panel of assays includes at least one migration assay.

In some embodiments, the panel of assays includes at least one adhesion assay.

In some embodiments, the migration assay is an in vitro wound closure assay.

In some embodiments, the in vitro wound closure assay is selected from the group consisting of a “scratch assay” (also referred to as a “scratch wound assay”), a circular scratch wound method, a circular scratch wound assay, and a circular wound closure assay.

In some embodiments, the panel of assays includes a reactive oxygen species (ROS) measurement.

In some embodiments, the ROS measurement assay is an in vitro assay using antioxidants as a positive control. In some embodiments, the ROS measurement assay is an in vitro assay using an antioxidant as a positive control. In some embodiments, N-acetylcysteine (NAC) or Ebselen is used as a positive control. Non-limiting examples of the antioxidants include vitamins and analogs thereof, including vitamin A, vitamin B3 (e.g., niacin [nicotinic acid] and nicotinamide), vitamin C (ascorbic acid), vitamin E (including tocopherols [e.g., α-tocopherol] and tocotriernols), and vitamin E analogs (e.g., trolox [water-soluble]); carotenoids, including carotenes (e.g., β-carotene), xanthophylls (e.g., lutein, zeaxanthin and meso-zeaxanthin), and carotenoids in saffron (e.g., crocin and crocetin); sulfur-containing antioxidants, including glutathione (GSH), N-acetyl-L-cysteine (NAC), bucillamine, S-nitroso-N-acetyl-L-cysteine (SNAC), S-allyl-L-cysteine (SAC), S-adenosyl-L-methionine (SAM), α-lipoic acid and taurine; scavengers of ROS and radicals, including carnosine, N-acetylcarnosine, curcuminoids (e.g., curcumin, demethoxycurcumin and tetrahydrocurcumin), cysteamine, Ebselen, glutathione, hydroxycinnamic acids and derivatives (e.g., esters and amides) thereof (e.g., caffeic acid, rosmarinic acid and tranilast), melatonin and metabolites thereof, nitrones (e.g., disufenton sodium [NXY-059]), nitroxides (e.g., XJB-5-131), polyphenols (e.g., flavonoids [e.g., apigenin, genistein, luteolin, naringenin and quercetin]), superoxide dismutase mimetics (infra), tirilazad, vitamin C, vitamin E and analogs thereof (e.g., α-tocopherol and trolox), and xanthine derivatives (e.g., pentoxifylline); mitochondrial antioxidants/“vitamins”, including ubiquinone (coenzyme Q, such as CoQ10), ubiquinol (a reduced and more bioavailable form of ubiquinone, such as ubiquinol-10), ubiquinone/ubiquinol analogs (e.g., idebenone and mitoquinone) and derivatives; mitochondria-targeted antioxidants, including DMQ, DMMQ, MitoE, MitoQ, Mito-TEMPO, MitoVitE, and the SkQ class of compounds (e.g., SkQ1, SkQ2, SkQ3, SkQB, SkQR1, SkQT, SkQT1, SkQT1(m), SkQT1(p), SkQTK1, SkQTR1, SkQBerb and SkQPalm); inhibitors of enzymes that produce ROS, including NADPH oxidase (NOX) inhibitors (e.g., apocynin, decursin and decursinol angelate [both inhibit NOX-1, -2 and -4 activity and expression], diphenylene iodonium, and GKT-831 [formerly GKT-137831, a dual NOX1/4 inhibitor]), NADH:ubiquinone oxidoreductase (complex I) inhibitors (e.g., metformin and rotenone), xanthine oxidase inhibitors (e.g., allopurinol, oxypurinol, tisopurine, febuxostat, topiroxostat, myo-inositol, phytic acid, and flavonoids [e.g., kaempferol, myricetin and quercetin]), and myeloperoxidase inhibitors (e.g., azide, 4-aminobenzoic acid hydrazide and PF-06667272, and apoE mimetics such as AEM-28 and AEM-28-14); substances that mimic or increase the activity or production of antioxidant enzymes, including superoxide dismutase (SOD) (e.g., SOD mimetics such as manganese (III)- and zinc (III)-porphyrin complexes (e.g., MnTBAP, MnTMPyP and ZnTBAP), manganese (II) penta-azamacrocyclic complexes (e.g., M40401 and M40403), manganese (III)-salen complexes (e.g., those disclosed in U.S. Pat. No. 7,122,537, incorporated herein by reference in its entirety) and OT-551 (a cyclopropyl ester prodrug of tempol hydroxylamine), and resveratrol and apoA-I mimetics such as 4F (both increase expression)), catalase (e.g., catalase mimetics such as manganese (III)-salen complexes [e.g., those disclosed in U.S. Pat. No. 7,122,537], and zinc [increases activity]), glutathione peroxidase (GPx) (e.g., apomorphine and zinc [both increase activity], and beta-catenin, etoposide and resveratrol [all three increase expression]), glutathione reductase (e.g., 4-tert-butylcatechol and redox cofactors such as flavin adenine dinucleotide [FAD] and NADPH [all three enhance activity]), glutathione S-transferase (GST) (e.g., phenylalkyl isothiocyanate-cysteine conjugates (e.g., S—[N-benzyl(thiocarbamoyl)]-L-cysteine), phenobarbital, rosemary extract and carnosol [all enhance activity]), thioredoxin (Trx) (e.g., geranylgeranylacetone, prostaglandin E1 and sulforaphane [all increase expression]), NADPH-quinone oxidoreductase 1 (NQO1) (e.g., flavones [e.g., β-naphthoflavone (5,6-benzoflavone)] and triterpenoids [e.g., oleanolic acid analogs such as TP-151 (CDDO), TP-155 (CDDO methyl ester), TP-190, TP-218, TP-222, TP-223 (CDDO carboxamide), TP-224 (CDDO monomethylamide), TP-225, TP-226 (CDDO dimethylamide), TP-230, TP-235 (CDDO imidazolide), TP-241, CDDO monoethylamide, CDDO mono(trifluoroethyl)amide, and (+)-TBE-B], all of which increase expression by activating Nrf2), heme oxygenase 1 (HO-1) (e.g., curcuminoids (e.g., curcumin), triterpenoids (e.g., oleanolic acid analogs such as TP-225), and apoA-I mimetics (e.g., 4F), all of which increase expression), and paraoxonase 1 (PON-1) (e.g., apoE mimetics [e.g., AEM-28 and AEM-28-14] and apoA-I mimetics [e.g., 4F], both types increasing activity); activators of transcription factors that upregulate expression of antioxidant enzymes, including activators of nuclear factor (erythroid-derived 2)-like 2 (NFE2L2 or Nrf2) (e.g., bardoxolone methyl, OT-551, fumarates (e.g., dimethyl and monomethyl fumarate), dithiolethiones (e.g., oltipraz), flavones (e.g., β-naphthoflavone), isoflavones (e.g., genistein), sulforaphane, trichostatin A (also upregulates glutathione synthesis), triterpenoids (e.g., oleanolic acid analogs [e.g., TP-225]), and melatonin (increases Nrf2 expression)); other kinds of antioxidants, including anthocyanins, benzenediol abietane diterpenes (e.g., carnosic acid), cyclopentenone prostaglandins (such as 15d-PGJ2, which also upregulate glutathione synthesis), flavonoids (e.g., flavonoids in Ginkgo biloba (e.g., myricetin and quercetin [increases levels of GSH, SOD, catalase, GPx and GST]), prenylflavonoids (e.g., isoxanthohumol), flavones (e.g., apigenin), isoflavones (e.g., genistein), flavanones (e.g., naringenin) and flavanols (e.g., catechin and epigallocatechin-3-gallate)), omega-3 fatty acids and esters thereof (supra), phenylethanoids (e.g., tyrosol and hydroxytyrosol), retinoids (e.g., all-trans retinol [vitamin A]), stilbenoids (e.g., resveratrol), uric acid, apoA-I mimetics (e.g., 4F), apoE mimetics (e.g., AEM-28 and AEM-28-14), and minerals (e.g., selenium and zinc [e.g., zinc monocysteine]); and analogs, derivatives and salts thereof.

In some embodiments, in vitro ROS measurement assay is evaluated using chloromethyl modified H₂DCFDA (CM-H₂DCFDA) and/or MitoSOX Red.

In some embodiments, the panel of assays includes a cell viability assay.

In some embodiments, the cell viability assay is the Microtiter Tetrazolium (MTT) assay.

In some embodiments, the cells are subject to an oxidative stress assay.

In some embodiments, the oxidative stress assay comprises:
a) inducing oxidative stress in cells; and
b) evaluating the extent of the oxidative stress.

In some embodiments, the oxidative stress is induced by one or more inducing agents selected from the group consisting of ketocholesterol, FeCl₃-sodium nitrilotriacetate (Fe-NTA), H₂O₂, tert-butyl hydroperoxide (t-BHP), all-trans retinal, NaIO4, hydroquinone, and Oxidized Cholysterol (OxLDL).

In some embodiments, the oxidative stress is induced by culturing the cells under hypoxic and/or anoxic conditions.

In some embodiments, the oxidative stress is induced by administering to the assayed cells ketocholesterol at a concentration of about 0.1 to 1 mM, about 1 to 10 mM, or about 10 to 100 mM, for about 1 to 24 hours, about 24 to 48 hours, or about 48 to 72 hours.

In some embodiments, the oxidative stress is induced by administering to the assayed cells Fe-NTA at a concentration of about 0.1 to 1 mM, about 1 to 10 mM, or about 10 to 100 mM, for about 1 to 24 hours, about 24 to 48 hours, or about 48 to 72 hours.

In some embodiments, the oxidative stress is induced by administering to the assayed cells H₂O₂ at a concentration of about 0.01 to 0.1 μM, about 0.1 to 1 μM, about 1 to M, about 10 to 100 μM, or about 100 to 1000 μM, for about 0.5 to 6 hours, about 6 to 12 hours, about 12 to 18 hours, or about 18 to 24 hours.

In some embodiments, the oxidative stress is induced by administering to the assayed cells t-BHB at a concentration of about 0.01 to 0.1 μM, about 0.1 to 1 μM, about 1 to 10 μM, about 10 to 100 μM, or about 100 to 1000 μM, for about 0.5 to 6 hours, about 6 to 12 hours, about 12 to 18 hours, or about 18 to 24 hours.

In some embodiments, the oxidative stress is induced by administering to the assayed cells all-trans retinal at a concentration of about 0.01 to 0.1 μM, about 0.1 to 1 μM, about 1 to 10 μM, about 10 to 100 μM, or about 100 to 1000 μM, for about 0.5 to 6 hours, about 6 to 12 hours, about 12 to 18 hours, or about 18 to 24 hours.

In some embodiments, the oxidative stress is induced by administering to the assayed cells NaIO₄ at a concentration of about 0.01 to 0.1 μM, about 0.1 to 1 μM, about 1 to 10 μM, about 10 to 100 μM, or about 100 to 1000 μM, for about 0.5 to 6 hours, about 6 to 12 hours, about 12 to 18 hours, or about 18 to 24 hours.

In some embodiments, the oxidative stress is induced by administering to the assayed cells OxLDL at a concentration of about 10 to 100 μg/mL, about 10 to 100 μg/mL, or about 100 to 500 μg/mL, for about 0.5 to 6 hours, about 6 to 12 hours, about 12 to 18 hours, or about 18 to 24 hours.

In some embodiments, evaluating the extent of the oxidative stress comprises one or more of the following:
i) measuring DNA oxidation with anti-8-oxo-2′-deoxyguanosine (8-oxo-dG);
ii) measuring lipid oxidation with hiobarbituric acid reactive substances (TBARS);
iii) measuring cytoplasmic ROS with CM-H2 DCFDA;
iv) measuring mitochondrial ROS with Mitoxed; and
v) measuring mitochondrial potential such as measuring JC-1.

In some embodiments, the assayed cells is assayed for one or more phenotypes selected from the group consisting of:
alteration in epithelial polarization (for example, Na/K ATPase polarity), tight junctions (for example, ZO-1, trans-epithelial polarity, and trans-epithelial electrical resistance (TEER)), phagocytosis of outer segments, degradation of outer segments, phagosome composition and function, cathepsin D distribution activity, mitochondrial potential, inflammasome activation/activity, HIf-1 alpha activation/activity, apoptosis, ferroptosis, NRF-2 activation/activity, NOQ1 activation/activity, GPX4 activation/activity, SLC7AII activation/activity, Annexin V activation/activity, Propidium Iodine staining, IRE1/XBP1 activation/activity, PERK/ATF4/CHOP activation/activity, UPR activation/activity, ACS24 activation/activity, complement activation/susceptibility, lipid deposition, apoptosis, and necrosis.

In some embodiments, the MSC secretome is preconditioned before the oxidative stress is induced.

In some embodiments, the MSC secretome is preconditioned during the oxidative stress is induced.

In some embodiments, the MSC secretome is preconditioned after the oxidative stress is induced.

In some embodiments, preconditioning the MSC secretome comprises affecting secretory profile for the MSCs comprising one or more of the following: change in culture format (e.g., 2D planar vs. 3D bioreactor), different biomaterial scaffolds, co-culture, addition of pharmacological compounds, growth factors, chemokines, addition of toll-like receptor agonists, inflammatory cytokines, advanced glycation end products (AGEs), oxidized phospholipids, Malondialdehyde, or carboxyethylpyrrole, agitation presence of ECM, culture under sheer stress, agitation or suspension as aggregate or within a matrix, induced misfolded protein response, ER stress, induction of differentiation of the MSCs, culture in the presence of conditioned media and hypoxia/anoxia.

In some embodiments, preconditioning the MSC secretome comprises hypoxia preconditioning comprising culturing the MSCs in low oxygen culture environment.

In some embodiments, the oxygen level is about 0% to 2%.

In some embodiments, the hypoxia preconditioning is performed for about 4 to 12 hours, about 12 to 24 hours, about 24 to 36 hours, about 36 to 48 hours, about 48 to 60 hours, or about 60 to 72 hours.

In some embodiments, preconditioning the MSC secretome comprises treating the MSCs with one or more inflammatory cytokines.

In some embodiments, preconditioning the MSC secretome comprises treating the MSCs with one or more of the following: IL-6, PGE2, IDO, IFNγ, SDF-1, TGF-α, H₂O₂, FGF-2, IGF-1, BMP-2, atorvastatin, oxytocin, curcumin, lipopolysaccharide, and nicotinamide (NIC), vasoactive intestinal peptide (VIP) and/or diazoxide.

In some embodiments, preconditioning the MSC secretome comprises 3D culturing the MSCs (e.g., by using a 3D bioreactor).

In some embodiments, the present invention provides a bone marrow-derived mesenchymal stem cell (MSC) secretome composition comprising: HGF; Pentraxin-3 (TSG-14); VEGF; TIMP-1; Serpin E1; <5 ng/mL IL-8, and a tonicity modifying agent, wherein the MSC secretome is preconditioned.

In some embodiments, the MSC secretome composition further comprises:
i. at least one trophic factors/cytokines selected from the group consisting of TIMP-2, and VEGF-A;
ii. At least one additional factor selected from the group consisting of PEDF (Serpin F1), IGFBP-2, IGFBP-3, SDF-1, Kallikrein 3, MCP-1, Angiogenin, MCP-2, Angio-2, IL-6, IL-17, G-CSF, M-CSF, GM-CSF, IL-8, TNF-beta, and PDGF; and
iii. At least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and Thrombospondin-1.

In some embodiments, the MSC secretome composition comprises 1 ng/mL-100 ng/mL of at least one factor selected from the group consisting of Serpin E1, TIMP-1, Thrombospondin-1, Pentraxin-3 (TSG-14), and Serpin F1.

In some embodiments, the MSC secretome comprises 400 pg/mL-3000 pg/mL of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the MSC secretome composition further comprises at least one factor selected from the group consisting of Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and IFNγ.

In some embodiments, the MSC secretome composition comprises ratios of anti-angiogenic to pro-angiogenic wherein the ratio is >2, >3, >4, or >5.

In some embodiments, the MSC secretome composition comprises 1 pg/mL-400 pg/mL of VEGF.

In some embodiments, the level of VEGF is 5-10 fold lower than the level of Serpin E1.

In some embodiments, the composition comprises one or more anti-angiogenic factors, and wherein the ratio of the sum of the concentration of the one or more anti-angiogenic factors relative to the concentration of VEGF is >2, >3, >4, or >5.

In some embodiments, the MSC secretome comprises less than 1000 pg/mL of bFGF, PLGF, and PDGF.

In some embodiments, the MSC secretome composition has a pH of about 4.7 to about 7.5.

In some embodiments, the MSC secretome composition is formulated in a buffer system selected from the group consisting of di/mono sodium phosphate, sodium citrate/citric acid, boric acid/sodium citrate, boric acid/sodium tetraborate, and citric acid/disodium phosphate.

In some embodiments, the MSC secretome composition further comprises mono/di-sodium phosphate, mannitol, and trehalose, and wherein the composition has a pH of about pH 7.4.

In some embodiments, the MSC secretome composition further comprises divalent cations.

In some embodiments, the divalent cations are selected from the group consisting of Mg2+, Ca2+, and Zn2+.

In some embodiments, the MSC secretome composition further comprises di-sodium phosphate/citric acid, mannitol, and trehalose, wherein the composition has a pH of about pH 6.4.

In some embodiments, the MSC secretome composition further comprises an agent that increases viscosity.

In some embodiments, the adhesive agent is selected from the group consisting of hypromellose, Poloxamer 407, Poloxamer 188, Poloxomer 237, Poloxomer 338, Hypromellose, (HPMC), polycarbophil, polyvinylpyrrolidone (PVP), Polyvinyl alcohol (PVA), polyimide, sodium hyaluronate, gellan gum, poly(lactic acid-co-glycolic acid) (PLGA), polysiloxane, polyimide, carboxymethylcellulose (CMC), or hydroxypropyl methylcellulose (HPMC), hydroxy methyl cellulose, hydroxy ethyl cellulose, sodium carboxy methyl cellulose, fibrin glue, polyethyelene glycol, and GelCORE.

In some embodiments, the MSC secretome composition does not comprise one or more components selected from the group consisting of: xenobiotic components; Phenol red; peptides and biomolecules <3 kDa; antibiotics; protein aggregates >200 nm; cells; non-exosome/non-Extracellular Vesicles cell debris; hormones; and L-glutamine.

In some embodiments, the MSC secretome composition comprises:
i. 0.3-4.5 ng/mL HGF;
ii. 0.5-20 ng/mL Pentraxin-3 (TSG-14);
iii. 100-600 pg/mL VEGF;
iv. 10-200 ng/mL TIMP-1;
v. 20-80 ng/mL Serpin E1; and
vi. <5 ng/mL IL-8.

In some embodiments, the MSC secretome composition comprises an anti-angiogenic MSC secretome or an anti-scarring MSC secretome.

In some embodiments, the tonicity modifying agent is selected from the group consisting of NaCl, KCl, mannitol, dextrose, sucrose, sorbitol, and glycerin.

In some embodiments, the present invention provides a stable bone marrow-derived mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg hypromellose per mL;
and wherein the pH is about 4.7 to about 7.5, wherein the MSC secretome is preconditioned.

In some embodiments, the present invention provides a stable bone marrow-derived mesenchymal stem cell (MSC) secretome formulation comprising: i. 0.004%-0.08% w/w of MSC secretome; ii. 4%-5% w/w monobasic sodium phosphate; iii. 21.5%-23% w/w dibasic sodium phosphate; iv. 23%-25% w/w mannitol; v. 46%-48% w/w trehalose dehydrate; vi. 1%-3% w/w hypromellose; and wherein the pH is about 4.7 to about 7.5, wherein the MSC secretome is preconditioned.

In some embodiments, the present invention provides a method of treatment for an ocular condition in a subject in need thereof comprising administering to the subject a bone marrow-derived mesenchymal stem cell (MSC) secretome composition, wherein the MSC secretome composition comprises: HGF; Pentraxin-3 (TSG-14); VEGF; TIMP-1; Serpin E1; and <5 ng/mL IL-8, wherein the MSC secretome is preconditioned.

In some embodiments, the MSC secretome composition further comprises:
i. at least one trophic factors/cytokines selected from the group consisting of TIMP-2, and VEGF-A;
ii. At least one additional factor selected from the group consisting of PEDF (Serpin F1), IGFBP-2, IGFBP-3, SDF-1, Kallikrein 3, MCP-1, Angiogenin, MCP-2, Angio-2, IL-6, IL-17, G-CSF, M-CSF, GM-CSF, IL-8, TNF-beta, and PDGF; and
iii. At least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and Thrombospondin-1.

In some embodiments, the MSC secretome composition comprises 1 ng/mL-100 ng/mL of at least one factor selected from the group consisting of Serpin E1, TIMP-1, Thrombospondin-1, Pentraxin-3 (TSG-14), and Serpin F1.

In some embodiments, the MSC secretome composition comprises 400 pg/mL-3000 pg/mL of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the MSC secretome composition further comprises at least one factor selected from the group consisting of Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and IFNγ.

In some embodiments, the MSC secretome composition comprises ratios of anti-angiogenic to pro-angiogenic wherein the ratio is >2, >3, >4, or >5.

In some embodiments, the MSC secretome comprises 1 pg/mL-400 pg/mL of VEGF.

In some embodiments, the level of VEGF is 5-10 fold lower than the level of Serpin E1.

In some embodiments, the MSC secretome composition comprises one or more anti-angiogenic factor, and wherein the sum of the concentration of the one or more anti-angiogenic factors relative to the concentration of VEGF is >2, >3, >4, or >5.

In some embodiments, the MSC secretome comprises less than 1000 pg/mL of bFGF, PLGF, and PDGF.

In some embodiments, the MSC secretome composition has a pH of about 4.7 to about 7.5.

In some embodiments, the MSC secretome composition is formulated in a buffer system selected from the group consisting of di/mono sodium phosphate, sodium citrate/citric acid, boric acid/sodium citrate, boric acid/sodium tetraborate, and citric acid/disodium phosphate.

In some embodiments, the MSC secretome composition further comprises a tonicity modifying agent.

In some embodiments, the tonicity modifying agent is selected from the group consisting of NaCl, KCl, mannitol, dextrose, sucrose, sorbitol, and glycerin.

In some embodiments, the MSC secretome composition further comprises mono/di-sodium phosphate, mannitol, and trehalose, and wherein the composition has a pH of about pH 7.4.

In some embodiments, the MSC secretome composition further comprises divalent cations.

In some embodiments, the divalent cations are selected from the group consisting of Mg2+, Ca2+, and Zn2+.

In some embodiments, the MSC secretome composition further comprises di-sodium phosphate/citric acid, mannitol, and trehalose, and wherein the composition has a pH of about pH 6.4.

In some embodiments, the MSC secretome composition does not comprise one or more components selected from the group consisting of: xenobiotic components; Phenol red; peptides and biomolecules <3 kDa; antibiotics; protein aggregates >200 nm; cells; non-exosome/non-Extracellular Vesicles cell debris; hormones; and L-glutamine.

In some embodiments, the MSC secretome composition comprise an anti-angiogenic MSC secretome or an anti-scarring MSC secretome.

In some embodiments, the MSC secretome composition further comprises an agent that increases viscosity.

In some embodiments, the adhesive agent is selected from the group consisting of hypromellose, Poloxamer 407, Poloxamer 188, Poloxomer 237, Poloxomer 338, Hypromellose, (HPMC), polycarbophil, polyvinylpyrrolidone (PVP), Polyvinyl alcohol (PVA), polyimide, sodium hyaluronate, gellan gum, poly(lactic acid-co-glycolic acid) (PLGA), polysiloxane, polyimide, carboxymethylcellulose (CMC), or hydroxypropyl methylcellulose (HPMC), hydroxy methyl cellulose, hydroxy ethyl cellulose, sodium carboxy methyl cellulose, fibrin glue, polyethyelene glycol, and GelCORE.

In some embodiments, the MSC secretome composition comprises:
i. 0.3-4.5 ng/mL HGF;
ii. 0.5-20 ng/mL Pentraxin-3 (TSG-14);
iii. 100-600 pg/mL VEGF;
iv. 10-200 ng/mL TIMP-1;
v. 20-80 ng/mL Serpin E1; and
vi. <5 ng/mL IL-8.

In some embodiments, the present invention provides a method of treatment for an ocular condition in a subject in need thereof comprising administering to the subject a bone marrow-derived mesenchymal stem cell (MSC) secretome composition, wherein the MSC secretome composition is a stable mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg hypromellose per mL;
and wherein the pH is about 4.7 to about 7.5, wherein the MSC secretome is preconditioned.

In some embodiments, the present invention provides a method of treatment for an ocular condition in a subject in need thereof comprising administering to the subject a bone marrow-derived mesenchymal stem cell (MSC) secretome composition, wherein the MSC secretome composition is a stable mesenchymal stem cell (MSC) secretome formulation comprising:
i. 0.004%-0.08% w/w of MSC secretome
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate; and
vi. 1%-3% w/w hypromellose; and
wherein the pH is about 4.7 to about 7.5, wherein the MSC secretome is preconditioned.

In some embodiments, the present invention provides a composition that induces ocular wound healing comprising a mesenchymal stem cell (MSC) secretome and a tonicity modifying agent, wherein the MSC secretome is preconditioned, wherein the ability of the composition to promote ocular wound healing is indicated by a wound healing assay comprising:
a) providing a layer of corneal cells;
b) introducing a wound gap to the layer of corneal cells; and
c) determining whether the wound gap heals in the presence of the composition,
wherein the composition is administered to the corneal cells either before or after step b);
wherein closure of the wound gap is indicative of the ability of the composition to induce ocular wound healing.

In some embodiments, the corneal cells are corneal epithelial cells.

In some embodiments, the corneal cells are corneal keratocytes (or fibroblasts).

In some embodiments, the layer of corneal cells is a confluent monolayer.

In some embodiments, the wound gap is introduced by mechanically disrupting the layer of corneal cells.

In some embodiments, the wound gap is introduced by chemically disrupting the layer of corneal cells.

In some embodiments, the wound gap comprises a linear gap.

In some embodiments, the wound gap comprises a circular gap.

In some embodiments, the determining whether the wound gap closes in step c) comprises detecting and quantitating daily the migration and/or proliferation of the corneal cells within the wound gap.

In some embodiments, the migration and/or proliferation of the corneal cells is characterized as the number of corneal cells migrated and/or proliferated within the wound gap.

In some embodiments, determining whether the wound gap heals in step c) comprises measuring the size of the wound gap daily, wherein the size of the wound gap is expressed as a percentage of the initial size of the wound gap measured immediately after the wound gap is introduced.

In some embodiments, the size of the wound gap is characterized as the surface area of the wound gap.

In some embodiments, the size of the wound gap is characterized as the width of the wound gap.

In some embodiments, the step c) is performed within a period of 2-4 days after the completion of step b).

In some embodiments, the step c) is performed within a period of 2 days after the completion of step b).

In some embodiments, the step c) is performed within a period of 3 days after the completion of step b).

In some embodiments, the wound healing assay further comprises, prior to administering the composition to the corneal cells, a step of concentrating the composition, and optionally a step of buffer exchanging for the composition.

In some embodiments, the wound healing assay further comprises, prior to administering the composition to the corneal cells, a step of diluting the composition, and optionally a step of buffer exchanging for the composition.

In some embodiments, the composition comprises at least 45 μg/ml secretome proteins.

In some embodiments, the present invention provides a composition that induces ocular wound healing comprising a mesenchymal stem cell (MSC) secretome and a tonicity modifying agent, wherein the MSC secretome is preconditioned, wherein the ability of the composition to promote ocular wound healing is indicated by a wound healing assay comprising:
a) providing a layer of corneal cells;
b) introducing a wound gap to the layer of corneal cells; and
c) determining whether the wound gap heals in the presence of the composition,
wherein the composition is administered to the corneal cells either before or after step b);
wherein closure of the wound gap is indicative of the ability of the composition to induce ocular wound healing.

In some embodiments, the corneal cells are corneal epithelial cells.

In some embodiments, the corneal cells are corneal keratocytes (or fibroblasts).

In some embodiments, the layer of corneal cells is a confluent monolayer.

In some embodiments, the wound gap is introduced by mechanically disrupting the layer of corneal cells.

In some embodiments, the wound gap is introduced by chemically disrupting the layer of corneal cells.

In some embodiments, the wound gap comprises a linear gap.

In some embodiments, the wound gap comprises a circular gap.

In some embodiments, the determining whether the wound gap closes in step c) comprises detecting and quantitating daily the migration and/or proliferation of the corneal cells within the wound gap.

In some embodiments, the migration and/or proliferation of the corneal cells is characterized as the number of corneal cells migrated and/or proliferated within the wound gap.

In some embodiments, determining whether the wound gap heals in step c) comprises measuring the size of the wound gap daily, wherein the size of the wound gap is expressed as a percentage of the initial size of the wound gap measured immediately after the wound gap is introduced.

In some embodiments, the size of the wound gap is characterized as the surface area of the wound gap.

In some embodiments, the size of the wound gap is characterized as the width of the wound gap.

In some embodiments, the step c) is performed within a period of 2-4 days after the completion of step b).

In some embodiments, the step c) is performed within a period of 2 days after the completion of step b).

In some embodiments, the step c) is performed within a period of 3 days after the completion of step b).

In some embodiments, the wound healing assay further comprises, prior to administering the composition to the corneal cells, a step of concentrating the composition, and optionally a step of buffer exchanging for the composition.

In some embodiments, the wound healing assay further comprises, prior to administering the composition to the corneal cells, a step of diluting the composition, and optionally a step of buffer exchanging for the composition.

In some embodiments, the composition comprises at least 45 μg/ml secretome proteins.

In some embodiments, the present invention provides a method of preconditioning a MSC secretome, comprising affecting secretory profile for the MSCs comprising one or more of the following: change in culture format (e.g., 2D planar vs. 3D bioreactor), different biomaterial scaffolds, co-culture, addition of pharmacological compounds, growth factors, chemokines, addition of toll-like receptor agonists, inflammatory cytokines, advanced glycation end products (AGEs), oxidized phospholipids, Malondialdehyde, or carboxyethylpyrrole, agitation presence of ECM, culture under sheer stress, agitation or suspension as aggregate or within a matrix, induced misfolded protein response, ER stress, induction of differentiation of the MSCs, culture in the presence of conditioned media and hypoxia/anoxia.

In some embodiments, hypoxia preconditioning comprising culturing the MSCs in low oxygen culture environment.

In some embodiments, the oxygen level is about 0% to 2%.

In some embodiments, the hypoxia preconditioning is performed for about 4 to 12 hours, about 12 to 24 hours, about 24 to 36 hours, about 36 to 48 hours, about 48 to 60 hours, or about 60 to 72 hours.

In some embodiments, preconditioning the MSC secretome comprises treating the MSCs with one or more inflammatory cytokines.

In some embodiments, preconditioning the MSC secretome comprises treating the MSCs with one or more of the following: IL-6, PGE2, IDO, IFNγ, SDF-1, TGF-α, H₂O₂, FGF-2, IGF-1, BMP-2, atorvastatin, oxytocin, curcumin, lipopolysaccharide, and nicotinamide (NIC), vasoactive intestinal peptide (VIP) and/or diazoxide.

In some embodiments, preconditioning the MSC secretome comprises 3D culturing the MSCs (e.g., by using a 3D bioreactor).

In some embodiments, preconditioning the MSC secretome comprises inducing misfolded protein response in the MSCs.

In some embodiments, preconditioning the MSC secretome comprises culturing the MSCs under suitable conditions to induce ER stress.

In some embodiments, preconditioning the MSC secretome comprises culturing the MSCs under suitable conditions to induce differentiation of the MSCs into a mature retinal cell type or a precursor thereof.

In some embodiments, the present invention provides a bone marrow-derived mesenchymal stem cell (MSC) secretome composition comprising differentiating MSC to a pre-cursor of a mature retinal cell type or a mature retinal cell type. In some embodiments, the bone marrow-derived MSC secretome composition are obtained from an MSC culture, wherein the MSCs are cultured under conditions suitable to induce differentiation of the MSCs into a mature retinal cell type or a precursor thereof.

In some embodiments, the present invention provides a method of treating an ocular condition in a patient in need thereof, comprising administering to the patient the preconditioned MSC secretome disclosed herein.

In some embodiments, the present invention provides use of the preconditioned MSC secretome as disclosed herein for treating an ocular condition in a patient in need thereof.

In some embodiments, the ocular condition is selected from the group consisting of retinal condition, macular disease, Chronic Graft v. Host Disease (GvHD), Stevens-Johnson Syndrome, Ocular Mucous Membrane Pemphigoid, Persistent Corneal Epithelial Defect (PCED), limbal stem cell deficiency (LSCD), dry eye, ocular nerve tissue damage, and concussive injury to the eye (such as concussive injury, ocular contusion, or chemical burn).

### DETAILED DESCRIPTION OF THE INVENTION

### I. Introduction

The present invention provides methods, assays and protocols for mesenchymal stem cell secretome activity analysis, as well as uses for such characterized compositions. Such methods and assays are described in further detail below.

### A. Definitions

Terms used in the claims and specification are defined as set forth below unless otherwise specified. In the case of direct conflict with a term used in a parent provisional patent application, the term used in the instant specification shall control.

As used herein “isolated” refers to material removed from its original environment and is thus altered “by the hand of man” from its natural state.

As used herein, “enriched” means to selectively concentrate or to increase the amount of one or more materials by elimination of the unwanted materials or selection and separation of desirable materials from a mixture (e.g., separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers. As used herein, the term “multipotent stem cells” are true stem cells but can only differentiate into a limited number of types. For example, the bone marrow contains multipotent stem cells that give rise to all the cells of the blood but may not be able to differentiate into other cells types.

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Generally, clinical grade materials, such as recombinantly produced human proteins, are used in the preparation, growth, culturing, expansion, storage and/or formulation of such compositions and/or processes.

By the term “expanded”, in reference to cell compositions, means that the cell population constitutes a significantly higher concentration of cells than is obtained using previous methods. For example, the level of cells per gram of amniotic tissue in expanded compositions of AMP cells is at least 50-fold and up to 150-fold higher than the number of cells in the primary culture after 5 passages, as compared to about a 20-fold increase in such cells using previous methods. In another example, the level of cells per gram of amniotic tissue in expanded compositions of AMP cells is at least 30-fold and up to 100-fold higher than the number of cells in the primary culture after 3 passages. Accordingly, an “expanded” population has at least a 2-fold, and up to a 10-fold, improvement in cell numbers per gram of amniotic tissue over previous methods. The term “expanded” is meant to cover only those situations in which a person has intervened to elevate the number of the cells.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to, hormones, cytokines, extracellular matrix (ECM), proteins, vesicles, antibodies, chemokines, receptors, inhibitors and granules. The medium containing the cellular factors is the conditioned medium. Examples of methods of preparing conditioned media have been described in U.S. Pat. No. 6,372,494 which is incorporated by reference in its entirety herein. As used herein, conditioned medium also refers to components, such as proteins, that are recovered and/or purified from conditioned medium or from for example, MSC cells.

As used herein, the term “mesenchymal stem cell composition” or “MSC composition” means conditioned medium that has been derived from MSCs and in some instances has undergone further processing. In some embodiments, “MSC secretome” can refer to the crude conditioned media derived from the MSC. In some embodiments, “MSC secretome” can refer to the composition obtained from the crude conditioned media after it has been subjected to further processing as described herein.

As used herein, the term “suspension” means a liquid containing dispersed components, e.g., cytokines. The dispersed components may be fully solubilized, partially solubilized, suspended or otherwise dispersed in the liquid. Suitable liquids include, but are not limited to, water, osmotic solutions such as salt and/or sugar solutions, cell culture media, and other aqueous or non-aqueous solutions.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene. Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “secretome composition” refers to a composition comprising one or more substances which are secreted from a cell. In certain embodiments, a secretome composition may include one or more cytokines, one or more exosomes, and/or one or more microvesicles. A secretome composition may be purified or unpurified. In some embodiments, a secretome composition may further comprise one or more substances that are not secreted from a cell (e.g., culture media, additives, nutrients, etc.). In some a secretome composition does not comprise and or comprises only trace amounts of one or more substances that are not secreted from a cell (e.g., culture media, additives, nutrients, etc.).

The terms “treatment,” “treat,” or “treating,” and the like, as used herein covers any treatment of a human or nonhuman mammal (e.g., rodent, cat, dog, horse, cattle, sheep, and primates etc.), and includes preventing the disease or condition from occurring in a subject who may be predisposed to the disease or condition but has not yet been diagnosed as having it. It also includes inhibiting (arresting development of), relieving or ameliorating (causing regression of), or curing (permanently stopping development or progression) the disease, condition and/or any related symptoms. The terms “treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, e.g., arresting its development; (c) relieving and or ameliorating the disease or condition, e.g., causing regression of the disease or condition; or (d) curing the disease or condition, e.g., stopping its development or progression. The population of subjects treated by the methods of the invention includes subjects suffering from the undesirable condition or disease, as well as subjects at risk for development of the condition or disease. In some embodiments, “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder, and/or condition, and/or of a subject who exhibits only early signs of the disease, disorder, and/or condition. Alternatively and/or additionally, such treatment may be of a subject who exhibits one or more established signs of the relevant disease, disorder, and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, a “wound” is any disruption, from whatever cause, of normal anatomy (internal and/or external anatomy) including but not limited to traumatic injuries such as mechanical (e.g. contusion, penetrating), thermal, chemical, electrical, radiation, concussive and incisional injuries; elective injuries such as operative surgery and resultant incisional hernias, fistulas, etc.; acute wounds, chronic wounds, infected wounds, and sterile wounds, as well as wounds associated with disease states (e.g. ocular contusion). A wound is dynamic and the process of healing is a continuum requiring a series of integrated and interrelated cellular processes that begin at the time of wounding and proceed beyond initial wound closure through arrival at a stable wound closure. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scaring and/or the wounded area possesses tissue strength that is closer to that of uninjured tissue and/or the wounded tissue attains some degree of functional recovery.

As used herein, the terms “a” or “an” means one or more or at least one.

As used herein, a “therapeutically effective” or “effective” dosage or amount of a composition is an amount sufficient to have a positive effect on a given medical condition. If not immediate, the therapeutically effective or effective dosage or amount may, over period of time, provide a noticeable or measurable effect on a patient's health and well-being.

As used herein a “pharmaceutical composition” refers to an effective amount of the compositions described herein in combination with a delivery components. The pharmaceutical composition may optionally contain other components such as pharmaceutically suitable carriers and excipients, which may facilitate administration of a composition and/or its individual components to a subject.

The term “pharmaceutically acceptable carrier” refers to a carrier or a diluent that does not cause significant irritation to a subject and does not abrogate the biological activity and properties of the administered compounds.

The term “excipient” refers to an inert substance added to a pharmaceutical composition to further facilitate administration of a compound.

As used herein, the terms “mix”, “mixing”, and the like describe a mechanical process or a mechanical treatment of the components. For example, mixing can be in the sense of carrying out repeated cycles of pressing and folding or comparable processing steps which lead to an intense compression and mixing of the provided hydrophobic matrices.

Adult stem cells can be harvested from a variety of adult tissues, including bone marrow, fat, and dental pulp tissue. While all adult stem cells are cable of self-renewal and are considered multipotent, their therapeutic functions vary depending on their origin. As a result, each type of adult stem cell has unique characteristics that make them suitable for certain diseases. Mesenchymal stem cells (MSCs) are typically derived from the mesoderm and are multipotent, nonhematopoietic (non-blood) stem cells isolated from (derived from) capable of differentiating into a variety of tissues, including osteoblasts (e.g., bone cells), chondrocytes (e.g., cartilage cells), myocytes (e.g., muscle cells) and adipocytes (e.g., fat cells which give rise to marrow adipose tissue). As used herein, “isolated” refers to cells removed from their original environment. Stem cells produce factors, such as growth factors, that regulate or are important for regulating multiple biological processes. A growth factor is an agent, such as a naturally occurring substance capable of stimulating cellular growth and/or proliferation and/or cellular differentiation. Typically, growth factors are proteins or steroid hormones. While the terms “growth factor” and “factor” and the like are used interchangeably herein, the term “biological factor” is not limited to growth factors.

Human mesenchymal stem cells (MSCs), can be characterized by the surface marker profile of CD45−/CD31−/CD73+/CD90+/CD105+/CD44+(or any suitable subset thereof). (See Bourin et al., Cytotherapy 15(6):641-648 (2013)). Further, appropriate stem cells display the CD34+ positive at the time of isolation, but lose this marker during culturing. Therefore, the full marker profile for one stem cell type that may be used according to the present application includes CD45−/CD31−/CD73+/CD90+/CD105+. In another embodiment utilizing mouse stem cells, the stem cells are characterized by the Sca-1 marker, instead of CD34, to define what appears to be a homologue to the human cells described above, with the remaining markers remaining the same.

The phrase “conditioned medium” or “CM” refers to media which includes biological factors secreted by MSCs. This can also be referred to herein as the “secretome”, “MSC-CM”, “MSC secretome” and/or “MSC derived secretome”. Also provided are processed “conditioned medium” which included biological factors secreted by MSCs and which has been further processed by, for example, filtration, purification, and/or concentration procedures. The “conditioned medium” is obtained by culturing stem cells in media, as described herein in detail, and separating the resulting media, which contains stem cells and their secreted stem cell products (secretome) into conditioned medium that contains biological factors and fewer stem cells than were present prior to separation. The conditioned medium may be used in the methods described herein and is substantially free of stem cells (may contain a small percentage of stem cells) or free of stem cells. Biological factors that may be in the conditioned medium include, but are not limited to, proteins (e.g., cytokines, chemokines, growth factors, enzymes), nucleic acids (e.g., miRNA), lipids (e.g., phospholipids), polysaccharides, and/or combinations thereof. Any combination(s) of these biological factors may be either bound within or on the surface of extracellular vesicles (e.g., exosomes) or separate from extracellular vesicles.

The phrase “cellular pre-conditioning”, “MSC pre-conditioning’, or “secretome pre-conditioning” refers to any manipulation done to the MSCs or secretome-producing cells or secretome-producing cells prior to secretome harvest. Such manipulations include, but are not limited to change in culture format (e.g., 2D planar vs. 3D bioreactor), different biomaterial scaffolds, co-culture, addition of pharmacological compounds, growth factors, chemokines, addition of toll-like receptor agonists, inflammatory cytokines, Advanced glycation end products (AGEs), oxidized phospholipids, Malondialdehyde, or carboxyethylpyrrole, agitation presence of ECM, culture under sheer stress, agitation or suspension as aggregate or within a matrix, induced misfolded protein response, ER stress, induction of differentiation of the MSCs, culture in the presence of conditioned media and/or hypoxia/anoxia.

As used herein, the term “mature retinal cell” refers to a cell that may be contained in the retinal tissue of a human adult. Specific examples of the mature retinal cell include differentiated cells such as photoreceptor cells (rod and cone photoreceptor cells), bipolar cells, horizontal cells, amacrine cells, intervening nerve cells, retinal ganglion cells (ganglion cells), bipolar cells (rod bipolar cells, cone bipolar cells), Müller glial cells, retinal pigment epithelial (RPE) cells, and ciliary marginal zone cells.

As used herein, the term “retinal precursor cell” or “retinal progenitor cell” means a progenitor cell that has been determined to differentiate into a mature retinal cell. Non-limiting examples of the retinal precursor cell include photoreceptor progenitor cells, bipolar progenitor cells, retinal progenitor cells, horizontal progenitor cells, amacrine progenitor cells, retinal ganglion progenitor cells, Müller glial progenitor cells, and retinal pigment epithelial progenitor cells.

### B. Compositions and Formulations

According to the present description, compositions comprising conditioned medium comprising mesenchymal stem cell (MSC) secretome and/or mesenchymal stem cell (MSC) secretome (including processed MSC secretome) are provided herein.

In some embodiments, the MSC secretome is generally low for angiogenic factors. In some embodiments, the MSC secretome does not promote angiogenesis. In some embodiments, the MSC secretome exhibits anti-angiogenic properties. In some embodiments, the MSC secretome provides for reduced angiogenesis as compared to other secretome. In some embodiments, the MSC secretome provides for a 10%, 20%, 30%, 40%, 50%, 60%, 70%, 80%, 90%, 95%, or 99% reduction in angiogenesis. In some embodiments, the MSC secretome provides for a 10%, 20%, 30%, 40%, 50%, 60%, 70%, 80%, 90%, 95%, or 99% reduction in angiogenesis as compared to another secretome. In some embodiments, the MSC secretome provides for a 10%, 20%, 30%, 40%, 50%, 60%, 70%, 80%, 90%, 95%, or 99% reduction in angiogenesis as compared to the conditioned media prior to processing into the MSC secretome. In some embodiments, the MSC secretome has low angiogenesis induction. In some embodiments, the MSC secretome has reduced angiogenic response. In some embodiments, the MSC secretome has reduced angiogenic capacity. In some embodiments, the MSC secretome impairs and/or reduces the normal formation of blood vessels in presence of media supportive of angiogenesis. In some embodiments, the MSC secretome has reduced angiogenic capacity when the MSC secretome is compared to untreated control. In some embodiments, the MSC secretome has reduced angiogenic capacity as compared to a sample treated with serum containing media. In some embodiments, the MSC secretome attenuates an angiogenic response. In some embodiments, the MSC secretome reduces the angiogenic response induce by serum containing media. In some embodiments, a reduction in angiogenic response is induced by the MSC secretome when secretome plus serum containing media (reduced or no angiogenic response) is compared to serum containing media (angiogenic response). In some embodiments, an angiogenic response is indicated by tube formation in a cell based assay. In some embodiments, an angiogenic response is indicated by tube formation in an endothelial cell tube formation assay. In some embodiments, an angiogenic response is indicated by blood vessel formation in a CAM (Chick Chorioallantoic membrane) assay. In some embodiments, an angiogenic response is indicated by blood vessel formation in any blood vessel formation assay known in the art.

In some embodiments, the mesenchymal stem cell (MSC) secretome composition comprises:
i. IDO (Indoleamine-2,3-dioxygenase) enzyme activity;
ii. “threshold” ppm levels for at least one trophic factors/cytokines selected from the group consisting of HGF, FGF-7, TIMP-1, TIMP-2, PAI-1 (Serpin E1), VEGF-A, and b-NGF;
iii. “threshold” ppm levels for at least one additional factor selected from the group consisting of sFLT-1, PEDF (Serpin F1), IGFBP-2, IGFBP-3, SDF-1, TSG-14, Kallikrein 3, MCP-1, bFGF, Angiogenin, MCP-2, Angio-2, IL-6, IL-17, G-CSF, M-CSF, GM-CSF, IL-8, TNF-beta, PDGF, SOD1, SOD2, SOD3, and HO-1; and
iv. “threshold” ppm levels for at least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and Thrombospondin-1.

In some embodiments, the mesenchymal stem cell (MSC) secretome composition comprises:
i. less than about 250 μM IDO (Indoleamine-2,3-dioxygenase) enzyme activity;
ii. at least one trophic factors/cytokines selected from the group consisting of HGF, FGF-7, TIMP-1, TIMP-2, PAI-1 (Serpin E1), VEGF-A, and/or b-NGF;
iii. at least one additional factor selected from the group consisting of sFLT-1, PEDF (Serpin F1), IGFBP-2, IGFBP-3, SDF-1, TSG-14, Kallikrein 3, MCP-1, bFGF, Angiogenin, MCP-2, Angio-2, IL-6, IL-17, G-CSF, M-CSF, GM-CSF, IL-8, TNF-beta, PDGF, SOD1, SOD2, SOD3, and/or HO-1; and
iv. at least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and/or Thrombospondin-1.

A mesenchymal stem cell (MSC) secretome composition comprising:
i. at least one trophic factors/cytokines selected from the group consisting of HGF, TIMP-1, TIMP-2, PAI-1 (Serpin E1), VEGF-A, and b-NGF;
ii. at least one additional factor selected from the group consisting of PEDF (Serpin F1), IGFBP-2, IGFBP-3, SDF-1, TSG-14, Kallikrein 3, MCP-1, Angiogenin, MCP-2, Angio-2, IL-6, IL-17, G-CSF, M-CSF, GM-CSF, IL-8, TNF-beta, and PDGF; and
iii. at least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and Thrombospondin-1.

In some embodiments, the mesenchymal stem cell (MSC) secretome composition comprises at least one additional factor which includes but is not limited to Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and/or IFNγ.

In some embodiments, the mesenchymal stem cell (MSC) secretome composition comprises at least one additional factor selected from the group consisting of Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and IFNγ.

In some embodiments, the mesenchymal stem cell (MSC) secretome composition comprises at least one additional factor which includes but is not limited to a serpin family member, including serine protease inhibitors: Serpin F1, Serpin E1, Serpin A1, Serpin G1, Serpin H1, Serpin B6, Serpin E2, Serpin A3, Serpin C1, Serpin F2, Serpin I1), Serpin B1, Serpin B7, Serpin D1, Serpin B3, Serpin B8, Serpin B2, Serpin B12, Serpin A7, Serpin A4, and/or Serpin A6. In some embodiments, the mesenchymal stem cell (MSC) secretome composition comprises at least one additional factor which includes but is not limited to Serpin F1 (also referred to as PEDF), Serpin E1, and Serpin A1. In some embodiments, the mesenchymal stem cell (MSC) secretome composition comprises Serpin F1 (also referred to as PEDF). In some embodiments, the mesenchymal stem cell (MSC) secretome composition comprises Serpin E1. In some embodiments, the mesenchymal stem cell (MSC) secretome composition comprises Serpin A1.

In some embodiments, the mesenchymal stem cell (MSC) secretome composition comprises at least one additional factor which includes but is not limited to proteins involved in anti-oxidation. In some embodiments, the mesenchymal stem cell (MSC) secretome composition comprises at least one additional factor which includes but is not limited to Catalase, Protein disulfide-isomerase, Protein disulfide-isomerase A3, Protein disulfide-isomerase A4, Protein disulfide-isomerase A6, Peroxiredoxin-6, Peroxiredoxin-1, Peroxiredoxin-2, and/or Peroxiredoxin-4. In some embodiments, the mesenchymal stem cell (MSC) secretome composition comprises Catalase. In some embodiments, the mesenchymal stem cell (MSC) secretome composition comprises Protein disulfide-isomerase, In some embodiments, the mesenchymal stem cell (MSC) secretome composition comprises Protein disulfide-isomerase A3. In some embodiments, the mesenchymal stem cell (MSC) secretome composition comprises Protein disulfide-isomerase A4. In some embodiments, the mesenchymal stem cell (MSC) secretome composition comprises Protein disulfide-isomerase A6, In some embodiments, the mesenchymal stem cell (MSC) secretome composition comprises Peroxiredoxin-6. In some embodiments, the mesenchymal stem cell (MSC) secretome composition comprises Peroxiredoxin-1. In some embodiments, the mesenchymal stem cell (MSC) secretome composition comprises Peroxiredoxin-2. In some embodiments, the mesenchymal stem cell (MSC) secretome composition comprises Peroxiredoxin-4.

In some embodiments, the mesenchymal stem cell (MSC) secretome composition comprises at least one additional factor which includes but is not limited to a matrix metalloproteinases. In some embodiments, the mesenchymal stem cell (MSC) secretome composition comprises at least one additional factor which includes but is not limited to MMP2, MMP1, and/or MMP14. In some embodiments, the mesenchymal stem cell (MSC) secretome composition comprises MMP2. In some embodiments, the mesenchymal stem cell (MSC) secretome composition comprises MMP1. In some embodiments, the mesenchymal stem cell (MSC) secretome composition comprises MMP14.

In some embodiments, the mesenchymal stem cell (MSC) secretome composition comprises at least one additional factor which includes but is not limited to a protein selected from the group consisting of soluble scavenger receptor cysteine-rich domain-containing protein SSC5D, tumor necrosis factor-inducible gene 6 protein (aka TSG-6), serum albumin, and latent transforming growth factor binding protein (LTGFBP-1), including various isoforms, LTGFBP-2, LTGFBP-3, and LTGFBP-4. In some embodiments, the mesenchymal stem cell (MSC) secretome composition comprises soluble scavenger receptor cysteine-rich domain-containing protein SSC5D. In some embodiments, the mesenchymal stem cell (MSC) secretome composition comprises tumor necrosis factor-inducible gene 6 protein (aka TSG-6). In some embodiments, the mesenchymal stem cell (MSC) secretome composition comprises serum albumin. In some embodiments, the mesenchymal stem cell (MSC) secretome composition comprises LTGFBP-1. In some embodiments, the mesenchymal stem cell (MSC) secretome composition comprises LTGFBP-2. In some embodiments, the mesenchymal stem cell (MSC) secretome composition comprises LTGFBP-3. In some embodiments, the mesenchymal stem cell (MSC) secretome composition comprises LTGFBP-4.

In some embodiments, the mesenchymal stem cell (MSC) secretome composition comprises Pentraxin-3, TIMP-1, Serpin E1, TSP-1, and HGF.

In some embodiments, the mesenchymal stem cell (MSC) secretome composition comprises 2-16 ng/mL, or 9.8+/−0.5 ng/ml Pentraxin-3.

In some embodiments, the mesenchymal stem cell (MSC) secretome composition comprises 10-200 ng/mL, or 90+/−21.5 ng/ml TIMP-1.

In some embodiments, the mesenchymal stem cell (MSC) secretome composition comprises 10-100 ng/mL, or 49.2+/−9.8 ng/ml Serpin E1.

In some embodiments, the mesenchymal stem cell (MSC) secretome composition comprises 0.1-10 ng/mL, or 2.0+/−0.3 ng/ml HGF.

In some embodiments, the mesenchymal stem cell (MSC) secretome composition comprises 100-800 pg/mL, or 304+/−44 pg/ml VEGF.

In some embodiments, the mesenchymal stem cell (MSC) secretome composition comprises 0.1-100 pg/mL, or <1 ng/ml IL-8.

In some embodiments, the IDO (Indoleamine-2,3-dioxygenase) enzyme activity less than about 250 μM. In some embodiments, the IDO (Indoleamine-2,3-dioxygenase) enzyme activity is from 0 μM to about 250 μM. In some embodiments, the IDO (Indoleamine-2,3-dioxygenase) enzyme activity is from 50 μM to about 250 μM L-Kynurenine/million MSC. In some embodiments, the IDO (Indoleamine-2,3-dioxygenase) enzyme activity is from 50 μM to about 200 μM L-Kynurenine/million MSC. In some embodiments, the IDO (Indoleamine-2,3-dioxygenase) enzyme activity is from 100 μM to about 250 μM L-Kynurenine/million MSC. In some embodiments, the IDO (Indoleamine-2,3-dioxygenase) enzyme activity is from 100 μM to about 200 μM L-Kynurenine/million MSC. In some embodiments, the IDO (Indoleamine-2,3-dioxygenase) enzyme activity is about 0 μM, about 10 μM, about 20 μM, about 30 μM, about 40 μM, about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, about 200 μM, about 210 μM, about 220 μM, about 230 μM, about 240 μM, or about 250 μM L-Kynurenine/million MSC.

In some embodiments, the MSC secretome further comprises “threshold” ppm levels for at least one additional factor which includes but is not limited to sFLT-1, PEDF (Serpin F1), IGFBP-2, IGFBP-3, SDF-1, TSG-14, Kallikrein 3, MCP-1, bFGF, Angiogenin, MCP-2, Angio-2, IL-6, IL-17, G-CSF, M-CSF, GM-CSF, IL-8, TNF-beta, PDGF, SOD1, SOD2, SOD3, and/or HO-1. In some embodiments, the MSC secretome further comprises “threshold” ppm levels for at least one additional factor selected from the group consisting of sFLT-1, PEDF (Serpin F1), IGFBP-2, IGFBP-3, SDF-1, TSG-14, Kallikrein 3, MCP-1, bFGF, Angiogenin, MCP-2, Angio-2, IL-6, IL-17, G-CSF, M-CSF, GM-CSF, IL-8, TNF-beta, PDGF, SOD1, SOD2, SOD3, and HO-1. In some embodiments, the MSC secretome further comprises one additional factor in a concertation range of 200 pg/mL to 5000 pg/mL, wherein the one additional factor includes but is not limited to sFLT-1, PEDF (Serpin F1), IGFBP-2, IGFBP-3, SDF-1, TSG-14, Kallikrein 3, MCP-1, bFGF, Angiogenin, MCP-2, Angio-2, IL-6, IL-17, G-CSF, M-CSF, GM-CSF, IL-8, TNF-beta, PDGF, SOD1, SOD2, SOD3, and/or HO-1. In some embodiments, the MSC secretome further comprises 1000-3000 pg/mL of sFLT-1. In some embodiments, the MSC secretome further comprises 400-800 pg/mL of TSG-6.

In some embodiments, the MSC secretome further comprises 2000-8000 pg/mL of PEDF. In some embodiments, the MSC secretome further comprises 2000-7000 pg/mL of PEDF. In some embodiments, the MSC secretome further comprises 2000-6000 pg/mL of PEDF. In some embodiments, the MSC secretome further comprises 2000-5000 pg/mL of PEDF. In some embodiments, the MSC secretome further comprises 2000-4000 pg/mL of PEDF. In some embodiments, the MSC secretome further comprises 2000-3000 pg/mL of PEDF. In some embodiments, the MSC secretome further comprises 150-300 ng/mL PEDF. In some embodiments, the MSC secretome further comprises 200-300 ng/mL of PEDF. In some embodiments, the MSC secretome further comprises 200-275 ng/mL of PEDF. In some embodiments, the MSC secretome further comprises 225-275 ng/mL of PEDF. In some embodiments, the MSC secretome further comprises 150-300 ng/mL of PEDF. In some embodiments, the MSC secretome further comprises 273±27 ng/mL of PEDF.

In some embodiments, the MSC secretome further comprises “higher” levels of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome further comprises “higher” levels of Serpin E1. In some embodiments, the MSC secretome further comprises “higher” levels of Serpin A1. In some embodiments, the MSC secretome further comprises “higher” levels of TIMP-1. In some embodiments, the MSC secretome further comprises “higher” levels of Thrombospondin-1. In some embodiments, the MSC secretome further comprises “higher” levels of Pentraxin-3 (TSG-14). In some embodiments, the MSC secretome further comprises “higher” levels of Platelet Factor 4. In some embodiments, the MSC secretome further comprises “higher” levels of Serpin F1. In some embodiments, the MSC secretome comprises 1 ng/mL to 20 ng/mL at least one factor selected from the group consisting of Serpin E1, TIMP-1, Thrombospondin-1, Pentraxin-3 (TSG-14), Platelet Factor 4, and Serpin F1. In some embodiments, the MSC secretome comprises 1 ng/mL to 8 ng/mL at least one factor selected from the group consisting of Serpin E1, TIMP-1, Thrombospondin-1, Pentraxin-3 (TSG-14), Platelet Factor 4, and Serpin F1. In some embodiments, the MSC secretome comprises 2 ng/mL to 8 ng/mL at least one factor selected from the group consisting of Serpin E1, TIMP-1, Thrombospondin-1, Pentraxin-3 (TSG-14), Platelet Factor 4, and Serpin F1. In some embodiments, the MSC secretome comprises 3 ng/mL to 8 ng/mL at least one factor selected from the group consisting of Serpin E1, TIMP-1, Thrombospondin-1, Pentraxin-3 (TSG-14), Platelet Factor 4, and Serpin F1. In some embodiments, the MSC secretome comprises 4 ng/mL to 8 ng/mL at least one factor selected from the group consisting of Serpin E1, TIMP-1, Thrombospondin-1, Pentraxin-3 (TSG-14), Platelet Factor 4, and Serpin F1. In some embodiments, the MSC secretome comprises 5 ng/mL to 8 ng/mL at least one factor selected from the group consisting of Serpin E1, TIMP-1, Thrombospondin-1, Pentraxin-3 (TSG-14), Platelet Factor 4, and Serpin F1. In some embodiments, the MSC secretome comprises 6 ng/mL to 8 ng/mL at least one factor selected from the group consisting of Serpin E1, TIMP-1, Thrombospondin-1, Pentraxin-3 (TSG-14), Platelet Factor 4, and Serpin F1. In some embodiments, the MSC secretome comprises 2 ng/mL to 7 ng/mL at least one factor selected from the group consisting of Serpin E1, TIMP-1, Thrombospondin-1, Pentraxin-3 (TSG-14), Platelet Factor 4, and Serpin F1.

In some embodiments, the MSC secretome composition further comprises “mid-range” levels of at least one factor including but not limited to Angiopoietin-1, Angiopoietin-2, Amphiregulin, Endostatin, Endothelin-1, Thrombospondin-2, and Thrombospondin-1, Angiogenin, DPPIV (Dipeptidyl peptidase-4), IGFBP-3, and/or uPA. In some embodiments, the MSC secretome composition further comprises “mid-range” levels of at least one factor selected from the group consisting of Angiopoietin-1, Angiopoietin-2, Amphiregulin, Endostatin, Endothelin-1, Thrombospondin-2, and Thrombospondin-1, Angiogenin, DPPIV, IGFBP-3, and uPA. In some embodiments, the MSC secretome composition further comprises “mid-range” levels of at least one factor selected from the group consisting of Angiogenin, DPPIV, IGFBP-3, and uPA. In some embodiments, the MSC secretome composition further comprises about 200 pg/mL to about 800 pg/mL of at least one factor selected from the group consisting of Angiogenin, DPPIV, IGFBP-3, and uPA. In some embodiments, he MSC secretome composition further comprises about 200 pg/mL to about 700 pg/mL, about 300 pg/mL to about 800 pg/mL, about 200 pg/mL to about 500 pg/mL, or about 300 pg/mL to about 500 pg/mL of at least one factor selected from the group consisting of Angiopoietin-1, Angiopoietin-2, Amphiregulin, Endostatin, Endothelin-1, Thrombospondin-2, and Thrombospondin-1, Angiogenin, DPPIV, IGFBP-3, and uPA. In some embodiments, the MSC secretome composition further comprises about 200 pg/mL, about 300 pg/mL, about 400 pg/mL, about 500 pg/mL, about 600 pg/mL, about 700 pg/mL, or about 800 pg/mL of at least one factor selected from the group consisting of Angiopoietin-1, Angiopoietin-2, Amphiregulin, Endostatin, Endothelin-1, Thrombospondin-2, and Thrombospondin-1, Angiogenin, DPPIV, IGFBP-3, and uPA. In some embodiments, the MSC secretome composition comprises about 200 pg/mL to about 800 pg/mL, about 300 pg/mL to 800 pg/mL, about 200 pg/mL to about 500 pg/mL, or about 300 pg/mL to about 500 pg/mL of Angiogenin. In some embodiments, the MSC secretome composition further comprises about 200 pg/mL to about 800 pg/mL, about 300 pg/mL to about 800 pg/mL, about 200 pg/mL to about 500 pg/mL, or about 300 pg/mL to about 500 pg/mL of DPPIV. In some embodiments, the MSC secretome composition comprises about 200 pg/mL to about 800 pg/mL, about 300 pg/mL to about 800 pg/mL, about 200 pg/mL to 500 pg/mL, or about 300 pg/mL to about 500 pg/mL of IGFBP-3. In some embodiments, the MSC secretome composition comprises 200 pg/mL to about 800 pg/mL, about 300 pg/mL to 800 pg/mL, about 200 pg/mL to 500 pg/mL, or about 300 pg/mL to about 500 pg/mL of uPA.

In some embodiments, the MSC secretome further comprises “low” levels of VEGF. In some embodiments, the MSC secretome further comprises about 1 pg/mL of VEGF. In some embodiments, the MSC secretome further comprises about 10 pg/mL of VEGF. In some embodiments, the MSC secretome further comprises about 20 pg/mL of VEGF. In some embodiments, the MSC secretome further comprises about 30 pg/mL of VEGF. In some embodiments, the MSC secretome further comprises about 40 pg/mL of VEGF. In some embodiments, the MSC secretome further comprises about 50 pg/mL of VEGF. In some embodiments, the MSC secretome further comprises about 60 pg/mL of VEGF. In some embodiments, the MSC secretome further comprises about 70 pg/mL of VEGF. In some embodiments, the MSC secretome further comprises about 80 pg/mL of VEGF.

In some embodiments, the MSC secretome further comprises about 90 pg/mL of VEGF. In some embodiments, the MSC secretome further comprises about 100 pg/mL of VEGF. In some embodiments, the MSC secretome further comprises about 125 pg/mL of VEGF. In some embodiments, the MSC secretome further comprises about 150 pg/mL of VEGF. In some embodiments, the MSC secretome further comprises about 175 pg/mL of VEGF. In some embodiments, the MSC secretome further comprises 1 pg/mL to about 400 pg/mL of VEGF. In some embodiments, the MSC secretome further comprises 10 pg/mL to about 400 pg/mL of VEGF. In some embodiments, the MSC secretome further comprises 50 pg/mL to about 350 pg/mL of VEGF. In some embodiments, the MSC secretome further comprises 50 pg/mL to about 300 pg/mL of VEGF. In some embodiments, the MSC secretome further comprises 10 pg/mL to about 300 pg/mL of VEGF. In some embodiments, the MSC secretome further comprises 100 pg/mL to about 300 pg/mL of VEGF. In some embodiments, the MSC secretome further comprises less than about 200 pg/mL of VEGF. In some embodiments, the MSC secretome further comprises less than about 200 pg/mL of VEGF. In some embodiments, the MSC secretome further comprises 0 pg/mL to about 200 pg/mL of VEGF. In some embodiments, the MSC secretome further comprises 0 pg/mL to about 200 pg/mL of VEGF. In some embodiments, the MSC secretome further comprises 10 pg/mL to about 200 pg/mL of VEGF. In some embodiments, the MSC secretome further comprises 20 pg/mL to about 200 pg/mL of VEGF. In some embodiments, the MSC secretome further comprises 30 pg/mL to about 200 pg/mL of VEGF. In some embodiments, the MSC secretome further comprises 40 pg/mL to about 200 pg/mL of VEGF. In some embodiments, the MSC secretome further comprises 50 pg/mL to about 200 pg/mL of VEGF. In some embodiments, the MSC secretome further comprises 60 pg/mL to about 200 pg/mL of VEGF. In some embodiments, the MSC secretome further comprises 70 pg/mL to about 200 pg/mL of VEGF. In some embodiments, the MSC secretome further comprises 80 pg/mL to about 200 pg/mL of VEGF. In some embodiments, the MSC secretome further comprises 90 pg/mL to about 200 pg/mL of VEGF. In some embodiments, the MSC secretome further comprises 100 pg/mL to about 200 pg/mL of VEGF. In some embodiments, the MSC secretome further comprises 10 pg/mL to about 150 pg/mL of VEGF. In some embodiments, the MSC secretome further comprises 20 pg/mL to about 150 pg/mL of VEGF. In some embodiments, the MSC secretome further comprises 30 pg/mL to about 150 pg/mL of VEGF. In some embodiments, the MSC secretome further comprises 40 pg/mL to about 150 pg/mL of VEGF. In some embodiments, the MSC secretome further comprises 50 pg/mL to about 150 pg/mL of VEGF. In some embodiments, the MSC secretome further comprises 60 pg/mL to about 150 pg/mL of VEGF. In some embodiments, the MSC secretome further comprises 70 pg/mL to about 150 pg/mL of VEGF. In some embodiments, the MSC secretome further comprises 80 pg/mL to about 150 pg/mL of VEGF. In some embodiments, the MSC secretome further comprises 90 pg/mL to about 150 pg/mL of VEGF. In some embodiments, the MSC secretome further comprises 100 pg/mL to about 150 pg/mL of VEGF. In some embodiments, the MSC secretome further comprises 10 pg/mL to about 100 pg/mL of VEGF. In some embodiments, the MSC secretome further comprises 20 pg/mL to about 100 pg/mL of VEGF. In some embodiments, the MSC secretome further comprises 30 pg/mL to about 100 pg/mL of VEGF. In some embodiments, the MSC secretome further comprises 40 pg/mL to about 100 pg/mL of VEGF. In some embodiments, the MSC secretome further comprises 50 pg/mL to about 100 pg/mL of VEGF. In some embodiments, the MSC secretome further comprises 60 pg/mL to about 100 pg/mL of VEGF. In some embodiments, the MSC secretome further comprises 70 pg/mL to about 100 pg/mL of VEGF. In some embodiments, the MSC secretome further comprises 80 pg/mL to about 100 pg/mL of VEGF. In some embodiments, the MSC secretome further comprises 90 pg/mL to about 100 pg/mL of VEGF. In some embodiments, the MSC secretome further comprises 100 pg/mL to about 100 pg/mL of VEGF.

In some embodiments of the MSC secretome composition the level of VEGF is 5-10 fold lower than the level of Serpin E1. In some embodiments of the MSC secretome composition the level of VEGF is 6-10 fold lower than the level of Serpin E1. In some embodiments of the MSC secretome composition the level of VEGF is 7-10 fold lower than the level of Serpin E1. In some embodiments of the MSC secretome composition the level of VEGF is 8-10 fold lower than the level of Serpin E1. In some embodiments of the MSC secretome composition the level of VEGF is 9-10 fold lower than the level of Serpin E1. In some embodiments of the MSC secretome composition the level of VEGF is 5-fold lower than the level of Serpin E1. In some embodiments of the MSC secretome composition the level of VEGF is 6-fold lower than the level of Serpin E1. In some embodiments of the MSC secretome composition the level of VEGF is 7-fold lower than the level of Serpin E1. In some embodiments of the MSC secretome composition the level of VEGF is 8-fold lower than the level of Serpin E1. In some embodiments of the MSC secretome composition the level of VEGF is 9-fold lower than the level of Serpin E1. In some embodiments of the MSC secretome composition the level of VEGF is 10-fold lower than the level of Serpin E1.

In some embodiments, the MSC secretome composition does not comprise and/or comprises very low levels of bFGF, PLGF, and PDGF. In some embodiments, the MSC secretome composition comprises less than about 200 pg/mL, less than about 150 pg/mL, less than about 100 pg/mL, less than about 75 pg/mL, less than about 50 pg/mL, or less than about 25 pg/mL bFGF, PLGF, and/or PDGF. In some embodiments, the MSC secretome composition comprises less than about 200 pg/mL, less than about 150 pg/mL, less than about 100 pg/mL, less than about 75 pg/mL, less than about 50 pg/mL, or less than about 25 pg/mL bFGF, PLGF, and PDGF. In some embodiments, the MSC secretome composition does not comprise bFGF, PLGF, and/or PDGF. In some embodiments, the MSC secretome composition does not comprise bFGF, PLGF, and PDGF. In some embodiments, the MSC secretome composition comprises less than about 200 pg/mL, less than about 150 pg/mL, less than about 100 pg/mL, less than about 75 pg/mL, less than about 50 pg/mL, or less than about 25 pg/mL of bFGF. In some embodiments, the MSC secretome composition does not comprise bFGF. In some embodiments, the MSC secretome composition comprises less than about 200 pg/mL, less than about 150 pg/mL, less than about 100 pg/mL, less than about 75 pg/mL, less than about 50 pg/mL, or less than about 25 pg/mL of PLGF. In some embodiments, the MSC secretome composition does not comprise PLGF. In some embodiments, the MSC secretome composition comprises less than about 200 pg/mL, less than about 150 pg/mL, less than about 100 pg/mL, less than about 75 pg/mL, less than about 50 pg/mL, or less than about 25 pg/mL of PDGF. In some embodiments, the MSC secretome composition does not comprise PDGF. In some embodiments, the MSC secretome composition does not comprise bFGF. In some embodiments, the MSC secretome composition does not comprise PLGF. In some embodiments, the MSC secretome composition does not comprise PDGF. In some embodiments, the MSC secretome composition comprises very low levels of bFGF, PLGF, and PDGF. In some embodiments, the MSC secretome composition comprises very low levels of bFGF. In some embodiments, the MSC secretome composition comprises very low levels of PLGF. In some embodiments, the MSC secretome composition comprises very low levels of PDGF.

In some embodiments, the MSC secretome composition comprises Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and/or IFNγ.

In some embodiments, the MSC secretome further comprises “higher” levels of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 1 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 1 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 1 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 1 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 20 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 20 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 20 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 20 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 30 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 30 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 30 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 30 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 4 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 40 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 40 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 40 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 50 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 50 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 50 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 50 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 60 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 60 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 60 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 60 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 70 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 70 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 70 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 70 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 80 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 80 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 80 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 80 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 90 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 90 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 90 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 90 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 100 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 100 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 100 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 110 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 110 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 110 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 120 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 120 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 120 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 130 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 130 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 130 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 140 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 140 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 140 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 150 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 150 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 150 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 160 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 160 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 160 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 170 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 170 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 170 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 180 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 180 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 180 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 190 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 190 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 190 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 200 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 200 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1 In some embodiments, the MSC secretome composition comprises 210 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 210 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 220 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 220 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 230 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 230 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 240 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 240 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 250 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 250 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 260 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 260 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 270 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 270 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 280 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 280 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 290 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 290 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 310 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 320 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 330 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 340 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 350 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 360 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 370 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 380 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 390 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-90 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-80 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 20 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 30 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 40 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 50 ng/mL-100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-70 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-60 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition comprises 10 ng/mL-50 ng/mL of at least one factor selected from the group consisting of Serpin E1, Serpin A1, TIMP-1, Thrombospondin-1, Pentraxin-3 (TSG-14), Platelet Factor 4, and Serpin F1.

In some embodiments, the MSC secretome composition comprises:
i. 0.3-4.5 ng/mL HGF;
ii. 0.5-20 ng/mL Pentraxin-3 (TSG-14);
iii. 100-600 pg/mL VEGF;
iv. 10-200 ng/mL TIMP-1;
v. 20-80 ng/mL Serpin E1; and
vi. <5 ng/mL IL-8.

In some embodiments, the MSC secretome composition comprises:
i. 1.5-3.5 ng/mL HGF;
ii. 5-15 ng/mL Pentraxin-3 (TSG-14);
iii. 200-400 pg/mL VEGF;
iv. 50-120 ng/mL TIMP-1;
v. 30-70 ng/mL Serpin E1; and
vi. <3 ng/mL IL-8.

In some embodiments, the MSC secretome composition comprises:
i. 1.5-2.5 ng/mL HGF;
ii. 8-12 ng/mL Pentraxin-3 (TSG-14);
iii. 250-350 pg/mL VEGF;
iv. 70-110 ng/mL TIMP-1;
v. 30-70 ng/mL Serpin E1; and
vi. <2 ng/mL IL-8.

In some embodiments, the MSC secretome composition comprises:
i. 2.0+/−0.3 ng/mL HGF;
ii. 9.8+/−0.5 ng/mL Pentraxin-3 (TSG-14);
iii. 304+/−44 pg/mL VEGF;
iv. 90+/−20 ng/mL TIMP-1;
v. 49.2+/−10 ng/mL Serpin E1; and
vi. <1 ng/mL IL-8.

In some embodiments, the MSC secretome composition is formulated at a pH of about pH 4.5 to about pH 8. In some embodiments, the MSC secretome composition is formulated at a pH of about pH 4.7 to about pH 7.8. In some embodiments, the MSC secretome composition is formulated at a pH of about pH 5.0 to about pH 7.5. In some embodiments, the MSC secretome composition is formulated at a pH of about pH 5.5 to about pH 7.5. In some embodiments, the MSC secretome composition is formulated at a pH of about pH 6 to about pH 7.5.

In some embodiments, the MSC secretome composition is formulated at a pH of about pH 4.5, about pH 5.0, about pH 5.5, about pH 6.0, about pH 6.5, about pH 7.0, about pH 7.4, about pH 8.0. In some embodiments, the MSC secretome composition is formulated at a pH of about pH 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In some embodiments, the MSC secretome composition does not comprise certain components. In some embodiments, the MSC secretome composition does not comprise certain components found in cellular media. In some embodiments, the MSC secretome composition does not comprise one or more components selected from the group consisting of xenobiotic components (for example, animal serum); Phenol red; peptides and biomolecules <3 kDa; antibiotics; protein aggregates (for example, protein aggregates >200 nm); cells; cell debris (cell debris do not include exosomes/Extracellular Vesicles (EVs); for example, non-exosome, non-EV cell debris); hormones (for example, hormones include, but are not limited to insulin and/or hydrocortisone); and/or L-glutamine. In some embodiments, the MSC secretome composition does not comprise xenobiotic components. In some embodiments, the MSC secretome composition does not comprise Phenol red. In some embodiments, the MSC secretome composition does not comprise peptides and biomolecules <3 kDa. In some embodiments, the MSC secretome composition does not comprise antibiotics. In some embodiments, the MSC secretome composition does not comprise protein aggregates (for example, protein aggregates >200 nm). In some embodiments, the MSC secretome composition does not comprise cells. In some embodiments, the MSC secretome composition does not comprise cell debris (cell debris do not include exosomes/Evs; for example, non-exosome, non-EV cell debris). In some embodiments, the MSC secretome composition does not comprise hormones (for example, hormones include, but are not limited to insulin and/or hydrocortisone. In some embodiments, the MSC secretome composition does not comprise L-glutamine.

In some embodiments, the MSC secretome further comprises mannitol, lactose, sorbitol, xylitol, sucrose, trehalose, mannose, maltose, lactose, glucose, raffinose, cellobiose, gentiobiose, isomaltose, arabinose, glucosamine, fructose, dextrose, and/or combinations thereof. In some embodiments, the MSC secretome further comprises phosphate. In some embodiments, the phosphate source is sodium phosphate or potassium phosphate. In some embodiments, the phosphate source is sodium phosphate. In some embodiments, the phosphate source is potassium phosphate. In some embodiments, the MSC secretome further comprises mono/di-sodium phosphate, mannitol, and trehalose, wherein the composition has a pH of about pH 7.4.

In some embodiments, the MSC secretome composition can comprise one or more additional agents including but not limited to glycine, glycerol, sodium chloride, potassium chloride, and/or dextrose. In some embodiments, the MSC secretome composition can comprise one or more additional agents selected from the group consisting of glycine, glycerol, sodium chloride, potassium chloride, and dextrose. In some embodiments, the MSC secretome composition can comprise one or more additional agents selected from the group consisting of glycine and glycerol, and dextrose. In some embodiments, the MSC secretome composition can comprise one or more additional agents selected from the group consisting of sodium chloride and potassium chloride.

In some embodiments, the MSC secretome composition is formulated in a buffer system. In some embodiments, the MSC secretome composition is formulated in a buffer system including but not limited to di/mono sodium phosphate, sodium citrate/citric acid, boric acid/sodium citrate, boric acid/sodium tetraborate, and/or citric acid/disodium phosphate. In some embodiments, the MSC secretome composition is formulated in a buffer system selected from the group consisting of di/mono sodium phosphate, sodium citrate/citric acid, boric acid/sodium citrate, boric acid/sodium tetraborate, and/or citric acid/disodium phosphate. In some embodiments, the MSC secretome composition is formulated in a di/mono sodium phosphate buffer system. In some embodiments, the MSC secretome composition is formulated in sodium citrate/citric acid buffer system. In some embodiments, the MSC secretome composition is formulated in a boric acid/sodium citrate buffer system. In some embodiments, the MSC secretome composition is formulated in a boric acid/sodium tetraborate buffer system. In some embodiments, the MSC secretome composition is formulated in a citric acid/disodium phosphate buffer system.

In some embodiments, the phosphate source is sodium phosphate or potassium phosphate. In some embodiments, the phosphate source is sodium phosphate. In some embodiments, the phosphate source is potassium phosphate. In some embodiments, the MSC secretome composition comprises di-sodium phosphate/citric acid, mannitol, and trehalose, wherein the composition has a pH of about pH 6.4.

In some embodiments, the MSC secretome composition further comprises a tonicity adjusting or tonicity modifying agent. In some embodiments, tonicity adjusting or tonicity modifying agent includes but is not limited to NaCl, KCl, mannitol, dextrose, sucrose, sorbitol, and/or glycerin. In some embodiments, tonicity adjusting or tonicity modifying agent is selected from the group consisting of NaCl, KCl, mannitol, dextrose, sucrose, sorbitol, and/or glycerin.

In some embodiments, the MSC secretome composition further comprises an adhesive agent. In some embodiments, the MSC secretome composition further comprises an adhesive agent including but not limited to hypromellose, Poloxamer 407, Poloxamer 188, Poloxomer 237, Poloxomer 338, Hypromellose, (HPMC), HEC, polycarbophil, polyvinylpyrrolidone (PVP), PVA (polyvinyl alcohol, polyimide, sodium hyaluronate, gellan gum, poly(lactic acid-co-glycolic acid) (PLGA), polysiloxane, polyimide, carboxymethylcellulose (CMC), or hydroxypropyl methylcellulose (HPMC), hydroxy methyl cellulose, hydroxy ethyl cellulose, sodium carboxy methyl cellulose, fibrin glue, polyethyelene glycol, and GelCORE. In some embodiments, the adhesive agent is hypromellose. In some embodiments, the adhesive agent is fibrin glue. In some embodiments, the adhesive agent is a polyethyelene glycol. In some embodiments, the adhesive agent is GelCORE (see, Sani, et al., Science Advances, Vol. 5, no. 3 (2019)).

In some embodiments, the MSC secretome composition comprises (a) processed conditioned medium comprising the MSC secretome produced by any one of the methods described herein; and (b) a polymer. In some embodiments, the MSC secretome composition comprises conditioned medium comprising the MSC secretome which is produced as described herein and a polymer. In some embodiments, the MSC secretome composition comprises processed conditioned medium comprising the MSC secretome which is produced as described herein and a polymer. In some embodiments the polymer can be a biodegradable polymer from which the MSC secretome and/or processed MSC secretome components can be released. In some embodiments, the polymer enables sustained (slow) release of the MSC secretome components.

In some embodiments, the MSC secretome compositions provided herein are in the form of a therapeutic bandage (e.g., a polymer impregnated with MSC secretome composition). The therapeutic bandage may be configured as needed, depending on the application. In some embodiments, the bandage is in the form or a patch or is configured as mesh.

In some embodiments, the MSC secretome compositions exhibit bio-penetrance, for example, ocular penetration, corneal penetration, and/or corneal permeation. In some embodiments, the MSC secretome composition exhibits the ability to be absorbed by the eye. In some embodiments, the MSC secretome composition exhibits inherent bio-penetrance. In some embodiments, the MSC secretome composition exhibits excipient-enabled bio-penetrance. In some embodiments, the MSC secretome composition exhibits bio-penetrance due to upregulation of the smaller factors. In some embodiments, the MSC secretome composition exhibits bio-penetrance due to the presence of a biopreservative. In some embodiments, the MSC secretome composition exhibits bio-penetrance due to the presence of the biopreservative benzalkonium chloride.

In some embodiments, the MSC secretome compositions exhibit long half-life and/or have increased stability as compared to other treatments. In some embodiments, the MSC secretome compositions as provided herein allow for an upregulation of proteins that are allow for increased stability of the MSC secretome. In some embodiments, the MSC secretome compositions as provided herein allow for upregulating chaperone proteins to improve stability of other proteins in the MSC secretome.

In some embodiments, the MSC secretome compositions exhibit ultrapotency when administered to a subject in need thereof. In some embodiments, the MSC secretome compositions allow for therapeutic efficacy with one drop or one administration per day. C. METHODS OF PRODUCING/MANUFACTURING

According to the present invention, conditioned medium (and, thus, mesenchymal stem cell secreted factors) can be obtained from mesenchymal stem cells obtained from the patient or individual to be treated (the patient in need thereof) or from another (donor) individual, such as a young and/or healthy donor and/or from mesenchymal stem cells obtained commercially. For example, MSC obtained from the individual to be treated (autologous stem cells) or from a donor (allogeneic stem cells), can be used to produce the conditioned medium described herein, which can then be further processed into a MSC secretome composition as described herein. In some embodiments, MSCs can also be obtained from commercial suppliers. In some embodiments, commercially obtained MSCs can used in MSC secretome production.

According to the present invention, the method of making a anti-angiogenic mesenchymal stem cell (MSC) secretome composition comprising:
i. culturing mesenchymal stem cells (MSCs) in a first culture media;
ii. removing the first culture media from step (i) from the MSCs;
iii. washing the MSCs in step (ii);
iv. adding a second culture media and culturing for about 1-5 days;
v. harvesting the second culture media from step (iv) as conditioned media; and
vi. processing the conditioned media in step (v) into the MSC secretome composition as described herein.

In some embodiments, culturing can be performed using a bioreactor system for culturing cells. In some embodiments, culturing can be performed using a bioreactor system for culturing stem cells. In some embodiments, culturing can be performed using a bioreactor system for culturing mesenchymal stem cells. In some embodiments, culturing can be performed using a media mixing technology. In some embodiments, culturing can be performed using a PBS Vertical Wheel™ Mixing Technology.

In some embodiments, in step (iv) processing the conditioned media in step (v) into the secretome composition comprises:
a) filtering the harvested conditioned media from step (v) to remove cell particulate;
b) concentrating the filtered conditioned media from step (a); and
c) buffer exchanging with the formulation buffer.

In some embodiments, step c) comprises buffer exchanging with a buffer system selected from the group consisting of di/mono sodium phosphate, sodium citrate/citric acid, boric acid/sodium citrate, boric acid/sodium tetraborate, and citric acid/disodium phosphate.

In some embodiments, the filtering step (a) comprises the use of a 0.45 μm filter, a 0.22 μm filter, 0.8 μm filter, and 0.65 micron, a low protein binding PVDF membranes, and/or PES (polyethersulfone). In some embodiments, the filtering step (a) comprises the use of a 0.45 μm filter. In some embodiments, the filtering step (a) comprises the use of a 0.22 μm filter. In some embodiments, the filtering step (a) comprises the use of 0.8 μm filter. In some embodiments, the filtering step (a) comprises the use of 0.65 micron. In some embodiments, the filtering step (a) comprises the use of low protein binding PVDF membranes. In some embodiments, the filtering step (a) comprises the use of PES (polyethersulfone).

In some embodiments, the concentration step (b) comprises using a hollow fiber filters, tangential flow filtration systems, or centrifugation based size exclusion techniques. In some embodiments, the concentration step (b) comprises using a hollow fiber filters technique. In some embodiments, the concentration step (b) comprises using a tangential flow filtration systems. In some embodiments, the concentration step (b) comprises using a centrifugation based size exclusion technique.

In some embodiments, the centrifugation based size exclusion techniques employs a 3-10 kDa MW cutoff. In some embodiments, the centrifugation based size exclusion techniques employs at least a 3 kDa MW cutoff, at least a 4 kDa MW cutoff, at least a 5 kDa MW cutoff, at least a 6 kDa MW cutoff, at least a 7 kDa MW cutoff, at least a 8 kDa MW cutoff, at least a 9 kDa MW cutoff, at least a 10 kDa MW cutoff, at least a 11 kDa MW cutoff, at least a 12 kDa MW cutoff, at least a 13 kDa MW cutoff, at least a 14 kDa MW cutoff, at least a 15 kDa MW cutoff, at least a 16 kDa MW cutoff, at least a 17 kDa MW cutoff, at least a 18 kDa MW cutoff, at least a 19 kDa MW cutoff, at least a 20 kDa MW cutoff, at least a 21 kDa MW cutoff, at least a 22 kDa MW cutoff, at least a 23 kDa MW cutoff, at least a 24 kDa MW cutoff, at least a 25 kDa MW cutoff, at least a 26 kDa MW cutoff, at least a 27 kDa MW cutoff, at least a 28 kDa MW cutoff, at least a 29 kDa MW cutoff, and/or at least a 30 kDa MW cutoff.

In some embodiments, the method produces an MSC secretome composition and/or formulation as described herein above. In some embodiments, the first and/or second culture medium are MSC Media and/or MSC-XF.

MSCs, or cells differentiated from MSCs, can be made to produce a conditioned media comprising the desired secretome, e.g., which comprises desired cytokines and/or desired therapeutic properties as described herein. For example, the secretome can be produced from MSCs of a super donor cell line. The secretome can also be produced from MSCs obtained commercially. In come embodiments, allogeneic MSCs (and/or cells derived therefrom) and/or allogeneic MSC-derived secretome compositions can be prepared and stored for large groups of individuals. Allogeneic MSCs (and/or cells derived therefrom) and/or MSC-derived secretome compositions can be made in advance so that they are ready when people need them. In certain embodiments, MSCs (and/or cells derived therefrom) and/or MSC-derived secretome compositions can be processed to manufacture a more concentrated solution or composition (e.g., a mesenchymal stem cell derived secretome composition or MSC secretome composition as described herein).

In some embodiments, the initial culture medium and the first culture medium are different. In some embodiments, the initial culture medium and the first culture medium are the same. Non-limiting examples of cell culture medium or media useful in culturing MSCs to produce conditioned media comprising the MSC secretome according to the present invention include hMSC Media Booster XFM, hMSC High Performance Basal Media, Minimum Essential Medium Eagle (MEME), ADC-1, LPM (Bovine Serum Albumin-free), F10 (HAM), F12 (HAM), DCCM1, DCCM2, RPMI 1640, BGJ Medium (with and without Fitton-Jackson Modification), StemPro, MSCGro, MesenCult, NutriStem, Basal Medium Eagle (BME—with the addition of Earle's salt base), Dulbecco's Modified Eagle Medium (DMEM—with or without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Alpha (MEM-alpha), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred medium for use in the present invention is MEM-alpha. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62 72, edited by William B. Jakoby and Ira H. Pastan, published by Academic Press, Inc.

In some embodiments, the cell culture medium for mesenchymal stem cells can be a serum-free medium. In some embodiments, the cell culture medium for mesenchymal stem cells can be supplemented with serum. In some embodiments, the cell culture medium for mesenchymal stem cells can be supplemented human platelet lysate. In some embodiments, the serum can include fetal bovine serum (FBS). In some embodiments, the cell culture medium for mesenchymal stem cells can be supplemented with serum such as fetal serum of bovine or other species. In some embodiments, the cell culture medium for mesenchymal stem cells can be supplemented with other components to facilitate cell growth and/or promote cell health, such as mercaptoethanol and/or antibiotics. In some embodiments, the cell culture medium for mesenchymal stem cells is not supplemented with antibiotics.

In some embodiments, the oxygen percentage is varied to facilitate cell growth and/or promote cell health. In some embodiments, the oxygen is at 5%, 10%, 15%, 20%, or 25% volume to facilitate cell growth and/or promote cell health. In some embodiments, the mesenchymal stem cells are grown under partial oxygen pressure to facilitate cell growth and/or promote cell health. In some embodiments, the mesenchymal stem cells are grown under a low oxygen partial pressure environment to facilitate cell growth and/or promote cell health.

In one aspect, the present invention is directed to conditioned medium (CM) comprising biological factors secreted by mesenchymal stem cells, which can be referred to as conditioned media comprising the MSC secretome. The conditioned medium can be obtained by culturing mesenchymal stem cells in media, as described herein, and separating the resulting media, which contains mesenchymal stem cells and their secreted mesenchymal stem cell products (referred to as biological factors and/or the secretome) into the components parts of the conditioned medium contain the secretome and mesenchymal stem cells grown in the conditioned media. The conditioned medium once separated comprises the mesenchymal stem cell secretome and can be further processed and/or used according to the methods described herein and is substantially free of mesenchymal stem cells (may contain a small percentage of stem cells and/or trace amounts of stem cells) or free of mesenchymal stem cells. The MSC secretome comprises a variety of biological factors including hormones, cytokines, extracellular matrix, proteins, vesicles, antibodies, chemokines, receptors, inhibitor, and granules. As described herein, the conditioned medium or media (CM or conditioned media comprising the MSC secretome) comprising the MSC secretome can be further processed, producing concentrated, conditioned medium (pCM or concentrated MSC secretome).

In some embodiments, the conditioned media comprising the MSC secretome or concentrated MSC secretome is produced by culturing mesenchymal stem cells in culture medium, replacing culture medium in which the mesenchymal stem cells have been cultured. In some embodiments, the resultant conditioned media comprising the MSC secretome is harvested (collected), then processed to produce concentrated MSC secretome. In certain embodiments, processing of the harvested conditioned media comprising the MSC secretome includes removal of some, most, or essentially all of the medium, or removal of some, most, or essentially all of selected components of the conditioned medium.

In some embodiments, the harvested conditioned media comprising the MSC secretome is filtered to produce concentrated MSC secretome. In some embodiments, the harvested conditioned media comprising the MSC secretome is ultra-filtered to produce concentrated MSC secretome.

In one aspect, provided herein are methods of producing processed conditioned medium, comprising (a) culturing stem cells in a cell culture medium, thereby generating conditioned medium that comprises factors secreted by the mesenchymal stem cells (e.g., conditioned media comprising the mesenchymal stem cell secretome); (b) harvesting the conditioned medium thereby producing harvested conditioned medium (e.g., harvested mesenchymal stem cell secretome); and (c) filtering harvested conditioned medium (e.g., harvested mesenchymal stem cell secretome) to produce processed conditioned medium (mesenchymal stem cell secretome). In some embodiments, the stem cells of (a) are cultured (have been cultured) in growth medium prior to being cultured in growth factor-free medium. Thus, in some embodiments, the methods comprise: (a) culturing mesenchymal stem cells in a first growth medium; (b) replacing the first growth medium with a second growth medium and culturing the stem cells in the second growth medium, thereby generating conditioned media comprising the mesenchymal stem cell secretome; (c) harvesting the conditioned media comprising the mesenchymal stem cell secretome, thereby producing harvested conditioned medium comprising the mesenchymal stem cell secretome; and (d) filtering harvested conditioned medium to produce processed conditioned medium comprising the mesenchymal stem cell secretome.

In some embodiments, the MSC secretome of the present invention is further processed. In some embodiments, the MSC secretome of the present invention is further processed using techniques known in the art, including but not limited to extraction, freeze-thawing, homogenization, permeabilization, centrifugation, density gradient centrifugation, CsCl gradient centrifugation, iodixanol gradient centrifugation, ultracentrifugation, fractionation, precipitation, SDS-PAGE, native PAGE, size exclusion chromatography, liquid chromatography, gas chromatography, hydrophobic interaction chromatography, ion exchange chromatography, anion exchange chromatography, cation exchange chromatography, affinity chromatography, heparin sulfate affinity chromatography, sialic acid affinity chromatography, immunoaffinity chromatography, metal binding chromatography, nickel column chromatography, epitope tag purification, or lyophilization, or any combination thereof.

In some embodiments, the MSC secretome of the present invention is enriched by one or more of the following methods: affinity based enrichment, size based enrichment, cation or anion based enrichment, and fraction to enrich for favorable attributes.

In some embodiments, the stem cells are mesenchymal stem cells. Mesenchymal stem cells (MSCs) are multipotent (capable of differentiating into multiple, but not all, cell lineages) nonhematopoietic (non-blood) stem cells isolated from (derived from) a variety of adult tissues, including bone marrow and adipose tissue. In certain embodiments, the mesenchymal stem cells are isolated from bone marrow. “Isolated” refers to cells removed from their original environment. MSCs may differentiate into cells of mesodermal lineage, for example, adipocytes, osteoblasts, and chondrocytes. MSCs have a small cell body with few cell processes that are long and thin. The cell body contains a large, round nucleus with a prominent nucleolus, which is surrounded by finely dispersed chromatin particles, giving the nucleus a clear appearance. The remainder of the cell body contains a small amount of Golgi apparatus, rough endoplasmic reticulum, mitochondria, and polyribosomes. The cells, which are long and thin, are widely dispersed and the adjacent extracellular matrix is populated by a few reticular fibrils but is devoid of the other types of collagen fibrils [Brighton, et al. 1991 The Journal of Bone and Joint Surgery 73(6):832-47]. MSCs described herein may express the following molecular marker (protein molecule characteristic of plasma membrane of a cell or cell type) profiles: bone morphogenic protein receptor^{“1”} (BMPR⁺); CD34⁺Scal⁺Lin⁻; CD44⁺; c-kit⁺; Sca-1⁺; Thy-1⁺; NOTCH3; JAG1; ITGA11. MSCs may also express other cell type-specific markers (see, the World Wide Web at stemcells.nih.gov; Kaltz, et al. 2010 Exp Cell Res Oct 1; 316(16):2609-17, incorporated herein by reference). MSCs described herein may be identified based on colony-forming unit assays to detect the multipotent differentiation potential of the MSCs (to what cell types the MSCs give rise). However, cells that are somewhat differentiated (progenitor cells) can also be used.

i. MSC Secretome—Preconditioning

In some embodiments, in vitro pre-conditioning of secretome-producing MSCs can be employed to enhance the therapeutic capacity/potential of MSCs. In some embodiments, this enhancement occurs by affecting the secretory profile for the MSCs. In some embodiments, such pre-conditioning regimens can include but are not limited to change in culture format (e.g., 2D planar vs. 3D bioreactor), different biomaterial scaffolds, co-culture, addition of pharmacological compounds, growth factors, chemokines, addition of toll-like receptor agonists, inflammatory cytokines, advanced glycation end products (AGEs), oxidized phospholipids, malondialdehyde, or carboxyethylpyrrole, agitation presence of ECM, culture under sheer stress, agitation or suspension as aggregate or within a matrix, induced misfolded protein response, ER stress, induction of differentiation of the MSCs, culture in the presence of conditioned media and hypoxia/anoxia. See, Ferreira et al., 2018 Frontiers in Immunol. Vol. 9, Art. 2837, incorporated herein by reference.

The physiological oxygen tension in tissues varies from 1% in cartilage and bone marrow, to 12% in peripheral blood. The 21% oxygen levels routinely used in cell culture incubators are thus much higher than physiological conditions. In some embodiments, hypoxic preconditioning of MSCs enhances their regenerative and cytoprotective effects and/or proliferation rate and can increase the levels of cytoprotective molecules and exosome secretion in general. In some embodiments, the ability of MSCs to be able to switch from aerobic to anaerobic metabolism in vivo, can allow for cellular adaptation. In some embodiments, the MSCs can adapt to very low oxygen tension in vitro. In some embodiments, the low oxygen values can range from anoxia (0% O₂) to 2% O₂. In some embodiments, the low oxygen values can range from anoxia (0% O₂) to 2% O₂ during 4 hours to 72 hours, including 4 hours, 6 hours, 12 hours 18 hours, 24 hours, 36 hours, 48 hours, 60 hours, or 72 hours. In some embodiments, hypoxia-preconditioning can result in MSCs expressing factors associated to neovascularization. In some embodiments, hypoxia-preconditioning can result in MSCs expressing higher levels of HIF-1α, GDNF, BDNF, VEGF, Ang-1, SDF-1 and its receptor CXCR4, as well as EPO and its receptor EPOR. In some embodiments, hypoxia-preconditioning can result in MSCs expressing higher levels of neuroprotective factors and pro-angiogenic factors.

In some embodiments, the MSCs are stimulated with inflammatory cytokines. In some embodiments, the MSCs are stimulated with inflammatory cytokines which promote the production secretome comprising factors involved in the regulation of the immune response. In some embodiments, the MSCs are stimulated with inflammatory cytokines which result in secretomes comprising factors that promote chemoattraction of immune cells, modulation of inflammation, and/or enhancing migration/homing of other cells. Other immunoregulatory abilities of MSC secretome can include inhibition of NK cells, inhibition of complement system activation, monocyte differentiation towards M2 macrophages, suppression of cytotoxic T cell proliferation, and increase in regulatory T cell numbers. In some embodiments, the effector cytokines in these processes can include but are not limited to IL-6, PGE2, and IDO. In some embodiments, IFNγ pretreatment results in MSC secretomes with enhanced immunosuppressive abilities. In some embodiments, IFNγ pretreatment results in MSC secretomes with enhanced immunosuppressive abilities through IDO. In some embodiments, SDF-1 preconditioning can be employed as part of the preconditioning process. In some embodiments, SDF-1 preconditioning provides for MSC secretomes that result in increased angiogenesis and reduce fibrosis. In some embodiments, TGF-α preconditioned MSCs provides for secretomes with increased VEGF production. In some embodiments, melatonin preconditioned MSCs provides for MSC secretomes that result in greater cell survival under oxidative stress, induced misfolded protein response, or ER stress. In some embodiments, H₂O₂ preconditioned MSCs result in MSC secretomes with increased vascularization, higher survival rates, and reduced inflammation in a rat model of ischemia/reperfusion injury. MSCs can also be preconditioned with growth factor cocktails, namely FGF-2, IGF-1, and BMP-2 in order to generate MSC secretomes with these activities.

In some embodiments, the preconditioning approach is of a mechanical nature and involves mimicking the MSCs microenvironment through 3D culture methods. In some embodiments, a 3D cell culture container/bioreactor is employed. In some embodiments, a 3D culture of spheroids is employed, allowing for mimicry of physiological conditions within the bone marrow. In some embodiments, the spheroid cultures create a microenvironment where inner layers are exposed to much lower oxygen and nutrient levels, creating a hypoxic environment. In such embodiments, the MSCs express higher levels of TSG-6, SCT-1, LIF, IL-24, TRAIL, and CXCR4, thus allowing for the production of an MSC secretome with higher concentrations of these factors. In such embodiments, the MSC spheroid cultures show increased survival, proliferation and vascularization. In such embodiments, the MSCs produce MSC secretomes with enhanced immunomodulatory, angiogenic, antifibrotic, and anti-apoptotic activities.

A variety of pharmacological agents can also be used to precondition MSCs. In some embodiments, MSCs can be preconditioned with atorvastatin, oxytocin, curcumin, lipopolysaccharide, nicotinamide (NIC), vasoactive intestinal peptide (VIP) and/or diazoxide. In some embodiments, preconditioning MSCs with nicotinamide (NIC) and/or vasoactive intestinal peptide (VIP) to protect against oxidative stress can be performed. In some embodiments, a pharmaceutical composition based on a MSC secretome from MSCs preconditioned with the neuroprotective drugs VIP, NIC, or both, enhances the proliferative and neuroprotective effect of said secretome. See, Alonso-Alonso et al., (2020) Stem Cell Int Vol. 2020, Article ID 9463548, incorporated herein by reference.

In some embodiments, preconditioning the MSC secretome comprises inducing misfolded protein response in the MSCs.

In some embodiments, preconditioning the MSC secretome comprises culturing the MSCs under suitable conditions to induce ER stress.

In some embodiments, preconditioning the MSC secretome comprises culturing the MSCs under suitable conditions to induce differentiation of the MSCs into a mature retinal cell type or a precursor thereof.

In some embodiments, the retinal precursor cell includes but is not limited to photoreceptor progenitor cells, bipolar progenitor cells, retinal progenitor cells, horizontal progenitor cells, amacrine progenitor cells, retinal ganglion progenitor cells, Müller glial progenitor cells, and retinal pigment epithelial progenitor cells.

The present invention provides a preconditioned mesenchymal stem cell (MSC) secretome composition comprising:
i. less than about 250 μM IDO (Indoleamine-2,3-dioxygenase) enzyme activity;
ii. at least one trophic factors/cytokines selected from the group consisting of HGF, FGF-7, TIMP-1, TIMP-2, PAI-1 (Serpin E1), VEGF-A, and/or b-NGF;
iii. at least one additional factor selected from the group consisting of sFLT-1, PEDF (Serpin F1), Serpin A1, IGFBP-2, IGFBP-3, SDF-1, TSG-14, Kallikrein 3, MCP-1, bFGF, Angiogenin, MCP-2, Angio-2, IL-6, IL-17, G-CSF, M-CSF, GM-CSF, IL-8, TNF-beta, PDGF, SOD1, SOD2, SOD3, and/or HO-1; and
iv. at least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and/or Thrombospondin-1.

In some embodiments, the preconditioned MSC secretome further comprises “higher levels” of at least one factor selected from the group consisting of Serpin E1, Serpin A1, TIMP-1, Thrombospondin-1, Pentraxin-3 (TSG-14), Platelet Factor 4, and/or Serpin F1, optionally 1 ng/mL-8 ng/mL.

In some embodiments, the preconditioned MSC secretome further comprises “mid-range” levels of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and/or uPA, optionally 400 pg/mL-3000 pg/mL.

In some embodiments, the preconditioned MSC secretome further comprises at least one factor selected from the group consisting of Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and/or IFNγ.

In some embodiments, the preconditioned MSC secretome comprises ratios of anti-angiogenic to pro-angiogenic wherein the ratio is >2, >3, >4, or >5.

In some embodiments, the preconditioned MSC secretome further comprises “low” levels for VEGF, optionally 0 pg/mL-200 pg/mL.

In some embodiments, the level of VEGF is 5-10 fold lower than the level of Serpin E1.

In some embodiments, the composition comprises one or more anti-angiogenic factor, and wherein the sum of the concentration of the one or more anti-angiogenic factors relative to the concentration of VEGF is >2, >3, >4, or >5.

In some embodiments, the preconditioned MSC secretome does not comprise and/or comprises very low levels of bFGF, PLGF, and PDGF, optionally less than 1000 pg/mL.

In some embodiments, the preconditioned MSC secretome composition has a pH of about 4.7 to about 7.5.

In some embodiments, the preconditioned MSC secretome is formulated in a buffer system selected from the group consisting of di/mono sodium phosphate, sodium citrate/citric acid, boric acid/sodium citrate, boric acid/sodium tetraborate, and citric acid/disodium phosphate.

In some embodiments, the preconditioned MSC secretome composition further comprises a tonicity modifying agent.

In some embodiments, the tonicity modifying agent is selected from the group consisting of NaCl, KCl, mannitol, dextrose, sucrose, sorbitol, and glycerin.

In some embodiments, the preconditioned MSC secretome further comprises mono/di-sodium phosphate, mannitol, and trehalose, wherein the composition has a pH of about pH 7.4.

In some embodiments, the preconditioned MSC secretome further comprises divalent cations.

In some embodiments, the divalent cations are selected from the group consisting of Mg2+, Ca2+, and Zn2+.

In some embodiments, the preconditioned MSC secretome further comprises di-sodium phosphate/citric acid, mannitol, and trehalose, wherein the composition has a pH of about pH 6.4.

In some embodiments, the composition further comprises an adhesive agent.

In some embodiments, the adhesive agent is selected from the group consisting of hypromellose, Poloxamer 407, Poloxamer 188, Poloxomer 237, Poloxomer 338, Hypromellose, (HPMC), polycarbophil, polyvinylpyrrolidone (PVP), Polyvinyl alcohol (PVA), polyimide, sodium hyaluronate, gellan gum, poly(lactic acid-co-glycolic acid) (PLGA), polysiloxane, polyimide, carboxymethylcellulose (CMC), or hydroxypropyl methylcellulose (HPMC), hydroxy methyl cellulose, hydroxy ethyl cellulose, sodium carboxy methyl cellulose, fibrin glue, polyethyelene glycol, and GelCORE.

The present invention also provides a method of making a preconditioned mesenchymal stem cell (MSC) secretome composition comprising:
i. culturing mesenchymal stem cells (MSCs) in a first culture media;
ii. removing the first culture media from step (i) from the MSCs;
iii. washing the MSCs in step (ii);
iv. adding a second culture media and culturing for about 1-5 days;
v. harvesting the second culture media from step (iv) as conditioned media; and
vi. processing the conditioned media in step (v) into the preconditioned MSC secretome composition as described herein.

In some embodiments, the preconditioned MSC secretome composition is a secretome composition as described herein.

In some embodiments, step (vi) processing the conditioned media in step (v) into the secretome composition comprises:
a) filtering the harvested conditioned media from step (v) to remove cell particulate;
b) concentrating the filtered conditioned media from step (a); and
c) buffer exchanging with the formulation buffer.

In some embodiments, step c) comprises buffer exchanging with a buffer system selected from the group consisting of di/mono sodium phosphate, sodium citrate/citric acid, boric acid/sodium citrate, boric acid/sodium tetraborate, and citric acid/disodium phosphate.

In some embodiments, the filtering step (a) comprises the use of a 0.45 μm filter, a 0.22 μm filter, 0.8 μm filter, and 0.65 μm filter, a low protein binding PVDF membranes, and/or PES (polyethersulfone).

In some embodiments, the concentration step (b) comprises using a hollow fiber filters, tangential flow filtration systems, or centrifugation based size exclusion techniques.

In some embodiments, centrifugation based size exclusion techniques employs a 3-10 kDa MW cutoff.

In some embodiments, the present invention provides a method of treatment of an ocular disease comprising administering to a patient in need thereof therapeutically effective amount of a mesenchymal stem cell secretome composition as described herein or a composition made according to the methods described herein to a patient in need thereof.

In some embodiments, the composition is administered to a target area.

The present invention also provides a method for treating visual dysfunction following traumatic injury to ocular structures in a patient in need thereof comprising administering to the patient a therapeutically effective amount of a mesenchymal stem cell secretome composition as described herein or a composition made according to the methods described herein.

The present invention also provides a method for inducing and/or promoting ocular wound healing in a patient in need thereof comprising administering to the patient a therapeutically effective amount of a mesenchymal stem cell secretome composition as described herein or a composition made according to the methods described herein.

The present invention also provides a method for reducing and/or inhibiting neovascularization, reducing and/or inhibiting scarring, promoting and/or preserving vision, and/or increasing wound closure rate (e.g., decreasing would closure time) in a patient in need thereof comprising administering to the patient a therapeutically effective amount of a mesenchymal stem cell secretome composition as described herein or a composition made according to the methods described herein.

The present invention also provides a method for reducing and/or inhibiting neovascularization and reducing scarring in order to promote vision preservation in a patient in need thereof comprising administering to the patient a therapeutically effective amount of a mesenchymal stem cell secretome composition as described herein or a composition made according to the methods described herein.

In some embodiments, the mesenchymal stem cell secretome composition is formulated for topical administration.

In some embodiments, the mesenchymal stem cell secretome composition is formulated for subconjunctival injection.

In some embodiments, the mesenchymal stem cell secretome composition is formulated for intravitreal injection.

The present invention also provides a method for characterizing a MSC secretome, wherein the method comprises:
(i) subjecting an MSC secretome to one or more characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays; and
(ii) determining the results from the one or more assays in (i).

Exemplary disclosures of the characterization assays can be found in Oslowski C M et al., Methods Enzymol. 2011; 490:71-92, Wagstaff P E, et al., Int J Mol Sci. 2021 Jun. 30; 22(13):7081, Bandyopadhyay M, et al., Mol Vis. 2013 May 29; 19:1149-57, Murali A, et al., Clin Exp Ophthalmol. 2019 March; 47(2):274-285, Srinivasan B, et al., J Lab Autom. 2015; 20(2):107-26, Slijkerman R W, et al., Prog Retin Eye Res. 2015; 48:137-59, Artero Castro A, et al., Stem Cells. 2019 December; 37(12):1496-1504, and Weigle S, et al., J Biol Methods. 2019 Jun. 3; 6(2):e115, each of which is incorporated herein by reference in its entirety.

The present invention also provides a method determining biopotency and stability of a MSC secretome comprising, wherein the method comprises:
(i) subjecting an MSC secretome to one or more characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays; and
(ii) determining the results from the one or more assays in (i).

The present invention also provides a method for determining MSC secretome lot consistency between a plurality of MSC secretome lots, wherein the method comprises:
(i) subjecting an MSC secretome to one or more characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays; and
(ii) determining the results from the one or more assays in (i).

In some embodiments, the results in (ii) from a physical component characterization identify an anti-angiogenic MSC secretome as described herein.

In some embodiments, the results in (ii) from a safety analyses provides for a MSC secretome that exhibits blood compatibility, and low and/or no pyrogens and/or endotoxins.

In some embodiments, the results in (ii) from a stability assay provides for a MSC secretome that exhibits stability at 4° C., 20° C., and/or 25° C. (or room temperature) for at least 7 days.

In some embodiments, the results in (ii) from a proliferation assay provides for a MSC secretome that induces proliferation.

In some embodiments, the results in (ii) from a migration assay provides for a MSC secretome that induces migration.

In some embodiments, the results in (ii) from a adhesion assay provides for a MSC secretome that induces cell adhesion. Adhesion assays can be performed using techniques known in the art. Exemplary disclosures of adhesion assays are provided in US Patent Publication Nos. 20170067061 A1 and 20150050325 A1, and Blue et al., Blood 2008, 111, 1248, each incorporated herein by reference in its entirety.

In some embodiments, the results in (ii) from a neovascularization assay provides for a MSC secretome that inhibits or does not promote neovascularization.

In some embodiments, the results in (ii) from a differentiation/scarring assay provides for a MSC secretome that inhibits differentiation and/or scarring.

In some embodiments, the results in (ii) from an inflammation assay provides for a MSC secretome that inhibits inflammation or alters immune response.

In some embodiments, the method further comprises:
(iii) identifying a MSC secretome lot based on the results in (ii).

The present invention also provides a panel of tests and/or assays for characterizing a MSC secretome, wherein the panel comprises at least two characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, and/or inflammation assays.

The present invention also provides a panel of tests and/or assays for determining consistency between MSC secretome lots, wherein the panel comprises one or more characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, and/or inflammation assays.

In some embodiments, the physical component characterization identifies a MSC secretome as described herein.

In some embodiments, the results in (ii) from a safety analyses provides for a MSC secretome that exhibits blood compatibility, and low and/or no pyrogens and/or endotoxins.

In some embodiments, the stability assay identifies for a MSC secretome that exhibits stability at 4° C., 20° C., and/or 25° C. (or room temperature) for at least 7 days.

In some embodiments, the proliferation assay identifies for a MSC secretome that induces proliferation.

In some embodiments, the migration assay identifies a MSC secretome that induces migration.

In some embodiments, the neovascularization assay identifies for a MSC secretome that inhibits or does not promote neovascularization.

In some embodiments, the differentiation/scarring assay identifies a MSC secretome that inhibits differentiation and/or scarring.

In some embodiments, the inflammation assay identifies a MSC secretome that inhibits inflammation or alters immune response.

In some embodiments, the physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays are all performed.

In some embodiments, the panel of tests and/or assays as described herein identify a MSC secretome as described herein.

In some embodiments, the panel of tests and/or assays as described herein includes at least one migration assay. In some embodiments, the migration assay is an in vitro wound closure assay. In some embodiments, the in vitro wound closure assay is selected from the group consisting of a “scratch assay” (also referred to as a “scratch wound assay”), a circular scratch wound method, a circular scratch wound assay, and a circular wound closure assay. In some embodiments, the preconditioned MSC secretome is an anti-angiogenic MSC secretome and/or an anti-scarring MSC secretome.

In some embodiments, the preconditioned MSC secretome is an anti-angiogenic MSC secretome and/or or an anti-scarring MSC secretome.

In some embodiments, the preconditioned MSC secretome is an anti-angiogenic MSC secretome or an anti-scarring MSC secretome.

The present invention also provides a stable preconditioned mesenchymal stem cell (MSC) secretome formulation comprising:
i. 1-20 μg, optionally 2 μg-8 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg hypromellose per mL; and
wherein the pH is about 4.7 to about 7.5.

The present invention also provides a stable preconditioned mesenchymal stem cell (MSC) secretome formulation comprising:
i. 0.004%-0.0375%, optionally 0.008%-0.015% w/w of MSC secretome;
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w hypromellose; and
wherein the pH is about 4.7 to about 7.5.

The present invention also provides a stable preconditioned mesenchymal stem cell (MSC) secretome formulation comprising:
i. 6 μg of MSC secretome per mL;
ii. 2.28 mg monobasic sodium phosphate per mL;
iii. 11.45 mg dibasic sodium phosphate per mL;
iv. 12.2 mg mannitol per mL;
v. 24 mg trehalose dihydrate per mL;
vi. 1 mg hypromellose per mL; and
wherein the pH is about 7.4.

The present invention also provides a stable preconditioned mesenchymal stem cell (MSC) secretome formulation comprising:
i. 6 μg of MSC secretome per mL;
ii. 1.31 mg monobasic sodium phosphate per mL;
iii. 5.73 mg dibasic sodium phosphate per mL;
iv. 12.2 mg mannitol per mL;
v. 24 mg trehalose dihydrate per mL;
vi. 1 mg hypromellose per mL;
vii. 0.2 mg magnesium chloride per mL;
viii. 1.17 mg sodium chloride per mL; and
wherein the pH is about 7.4.

The present invention also provides a stable preconditioned mesenchymal stem cell (MSC) secretome formulation comprising:
i. 0.012% w/w of MSC secretome;
ii. 4.5% w/w monobasic sodium phosphate;
iii. 22.4% w/w dibasic sodium phosphate;
iv. 24% w/w mannitol;
v. 47.1% w/w trehalose dehydrate;
vi. 2.0% w/w hypromellose; and
wherein the pH is about 7.4.

The present invention also provides a stable preconditioned mesenchymal stem cell (MSC) secretome formulation comprising:
i. sodium phosphate 10 mM;
ii. histidine HCL 10 mM;
iii. trehalose dihydrate 10%;
iv. polysorbate 20 0.01%;
wherein the pH is about 5.5.

The present invention also provides a stable preconditioned mesenchymal stem cell (MSC) secretome formulation comprising:
i. sodium phosphate 10 mM
ii. sucrose 5%
iii. sodium chloride 10 mM
wherein the pH is about 6.2.

The present invention also provides a stable preconditioned mesenchymal stem cell (MSC) secretome formulation comprising:
i. sodium phosphate 10 mM
ii. sucrose 5.8%
iii. polysorbate 80 0.02%
wherein the pH is about 6.2.

The present invention also provides a preconditioned mesenchymal stem cell (MSC) secretome composition comprising:
i. at least one trophic factors/cytokines selected from the group consisting of HGF, TIMP-1, TIMP-2, PAI-1 (Serpin E1), VEGF-A, and b-NGF;
ii. at least one additional factor selected from the group consisting of PEDF (Serpin F1), Serpin A1, IGFBP-2, IGFBP-3, SDF-1, TSG-14, Kallikrein 3, MCP-1, Angiogenin, MCP-2, Angio-2, IL-6, IL-17, G-CSF, M-CSF, GM-CSF, IL-8, TNF-beta, and PDGF; and
iii. at least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and Thrombospondin-1.

In some embodiments, the preconditioned MSC secretome composition further comprises high levels of at least one factor selected from the group consisting of Serpin E1, Serpin A1, TIMP-1, Thrombospondin-1, Pentraxin-3 (TSG-14), Platelet Factor 4, and Serpin F1.

In some embodiments, the preconditioned MSC secretome composition comprises 1 ng/mL-400 ng/mL of at least one factor selected from the group consisting of Serpin E1, TIMP-1, Thrombospondin-1, Pentraxin-3 (TSG-14), Platelet Factor 4, and Serpin F1.

In some embodiments, the preconditioned MSC secretome composition further comprises mid-range levels of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the preconditioned MSC secretome composition 400 pg/mL-3000 pg/mL of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the preconditioned MSC secretome composition further comprises at least one factor selected from the group consisting of Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and IFNγ.

In some embodiments, the preconditioned MSC secretome composition comprises ratios of anti-angiogenic to pro-angiogenic wherein the ratio is >2, >3, >4, or >5.

In some embodiments of the preconditioned MSC secretome composition the anti-angiogenic factors includes one or more factors selected from the group consisting of PEDF, lower levels of VEGF, and Serpin E1 and the pro-angiogenic factors includes one or more factors selected from the group consisting of VEGF, Angiogenin, IGFBP-3, uPA, Angio-1, Angio-2, Endothelin-1.

In some embodiments, the preconditioned MSC secretome composition further comprises low levels for VEGF.

In some embodiments, the preconditioned MSC secretome composition comprises 1 pg/mL-400 pg/mL of VEGF.

In some embodiments of the preconditioned MSC secretome composition the level of VEGF is 5-10 fold lower than the level of Serpin E1.

In some embodiments, the preconditioned MSC secretome composition comprises one or more anti-angiogenic factor, and wherein the sum of the concentration of the one or more anti-angiogenic factors relative to the concentration of VEGF is >2, >3, >4, or >5.

In some embodiments, the preconditioned MSC secretome composition does not comprise and/or comprises very low levels of bFGF, PLGF, and PDGF.

In some embodiments, the preconditioned MSC secretome composition comprises less than 1000 pg/mL of bFGF, PLGF, and PDGF.

In some embodiments, the preconditioned MSC secretome composition has a pH of about 4.7 to about 7.5.

In some embodiments, the preconditioned MSC secretome composition is formulated in a buffer system selected from the group consisting of di/mono sodium phosphate, sodium citrate/citric acid, boric acid/sodium citrate, boric acid/sodium tetraborate, and citric acid/disodium phosphate.

In some embodiments, the preconditioned MSC secretome composition further comprises a tonicity modifying agent.

In some embodiments of the preconditioned MSC secretome composition the tonicity modifying agent is selected from the group consisting of NaCl, KCl, mannitol, dextrose, sucrose, sorbitol, and glycerin.

In some embodiments, the preconditioned MSC secretome composition further comprises mono/di-sodium phosphate, mannitol, and trehalose, and wherein the composition has a pH of about pH 7.4.

In some embodiments, the preconditioned MSC secretome composition further comprises divalent cations.

In some embodiments, the preconditioned MSC secretome composition the divalent cations are selected from the group consisting of Mg2+, Ca2+, and Zn2+.

In some embodiments, the preconditioned MSC secretome composition further comprises di-sodium phosphate/citric acid, mannitol, and trehalose, wherein the composition has a pH of about pH 6.4.

In some embodiments, the preconditioned MSC secretome composition further comprises an adhesive agent.

In some embodiments of the preconditioned MSC secretome composition the adhesive agent is selected from the group consisting of hypromellose, Poloxamer 407, Poloxamer 188, Poloxomer 237, Poloxomer 338, Hypromellose, (HPMC), polycarbophil, polyvinylpyrrolidone (PVP), Polyvinyl alcohol (PVA), polyimide, sodium hyaluronate, gellan gum, poly(lactic acid-co-glycolic acid) (PLGA), polysiloxane, polyimide, carboxymethylcellulose (CMC), or hydroxypropyl methylcellulose (HPMC), hydroxy methyl cellulose, hydroxy ethyl cellulose, sodium carboxy methyl cellulose, fibrin glue, polyethyelene glycol, and GelCORE.

In some embodiments, the preconditioned MSC secretome composition does not comprise one or more components selected from the group consisting of: xenobiotic components; Phenol red; peptides and biomolecules <3 kDa; antibiotics; protein aggregates >200 nm; cells; non-exosome/non-Extracellular Vesicles cell debris; hormones; and L-glutamine.

In some embodiments, the preconditioned MSC secretome composition comprises: HGF; Pentraxin-3 (TSG-14); VEGF; TIMP-1; Serpin E1; and <5 ng/mL IL-8.

In some embodiments, the preconditioned MSC secretome composition comprises:
i. 0.3-4.5 ng/mL HGF;
ii. 0.5-20 ng/mL Pentraxin-3 (TSG-14);
iii. 100-600 pg/mL VEGF;
iv. 10-200 ng/mL TIMP-1;
v. 20-80 ng/mL Serpin E1; and
vi. <5 ng/mL IL-8.

In some embodiments, the preconditioned MSC secretome composition comprises an anti-angiogenic MSC secretome or an anti-scarring MSC secretome.

The present invention also provides a stable preconditioned mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg hypromellose per mL; and
wherein the pH is about 4.7 to about 7.5.

The present invention also provides a stable preconditioned mesenchymal stem cell (MSC) secretome formulation comprising:
i. 0.004%-0.08% w/w of MSC secretome;
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w hypromellose; and
wherein the pH is about 4.7 to about 7.5.

The present invention further provides a method of treatment for an ocular condition in a subject in need thereof comprising administering to the subject a preconditioned mesenchymal stem cell (MSC) secretome composition, wherein the preconditioned MSC secretome composition comprises:
i. at least one trophic factors/cytokines selected from the group consisting of HGF, TIMP-1, TIMP-2, PAI-1 (Serpin E1), VEGF-A, and b-NGF;
ii. at least one additional factor selected from the group consisting of PEDF (Serpin F1), Serpin A1, IGFBP-2, IGFBP-3, SDF-1, TSG-14, Kallikrein 3, MCP-1, Angiogenin, MCP-2, Angio-2, IL-6, IL-17, G-CSF, M-CSF, GM-CSF, IL-8, TNF-beta, and PDGF; and
iii. at least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and Thrombospondin-1.

In some embodiments, the preconditioned MSC secretome composition further comprises high levels of at least one factor selected from the group consisting of Serpin E1, TIMP-1, Thrombospondin-1, Pentraxin-3 (TSG-14), Platelet Factor 4, and Serpin F1.

In some embodiments, the preconditioned MSC secretome composition comprises 1 ng/mL-100 ng/mL of at least one factor selected from the group consisting of Serpin E1, TIMP-1, Thrombospondin-1, Pentraxin-3 (TSG-14), Platelet Factor 4, and Serpin F1.

In some embodiments, the preconditioned MSC secretome composition further comprises mid-range levels of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the preconditioned MSC secretome composition comprises 400 pg/mL-3000 pg/mL of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the preconditioned MSC secretome composition further comprises at least one factor selected from the group consisting of Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and IFNγ.

In some embodiments, the preconditioned MSC secretome composition comprises ratios of anti-angiogenic to pro-angiogenic wherein the ratio is >2, >3, >4, or >5.

In some embodiments, the anti-angiogenic factors includes one or more factors selected from the group consisting of PEDF, lower levels of VEGF, and Serpin E1 and pro-angiogenic: VEGF, Angiogenin, IGFBP-3, uPA, Angio-1, Angio-2, Endothelin-1.

In some embodiments, the preconditioned MSC secretome composition further comprises low levels for VEGF.

In some embodiments, the preconditioned MSC secretome comprises 1 pg/mL -400 pg/mL of VEGF.

In some embodiments, the level of VEGF is 5-10 fold lower than the level of Serpin E1.

In some embodiments, the preconditioned MSC secretome composition comprises one or more anti-angiogenic factor, and wherein the sum of the concentration of the one or more anti-angiogenic factors relative to the concentration of VEGF is >2, >3, >4, or >5.

In some embodiments, the preconditioned MSC secretome composition does not comprise or comprises very low levels of bFGF, PLGF, and PDGF.

In some embodiments, the preconditioned MSC secretome composition comprises less than 1000 pg/mL of bFGF, PLGF, and PDGF.

In some embodiments, the preconditioned MSC secretome composition has a pH of about 4.7 to about 7.5.

In some embodiments, the preconditioned MSC secretome composition is formulated in a buffer system selected from the group consisting of di/mono sodium phosphate, sodium citrate/citric acid, boric acid/sodium citrate, boric acid/sodium tetraborate, and citric acid/disodium phosphate.

In some embodiments, the preconditioned MSC secretome composition further comprises a tonicity modifying agent.

In some embodiments, the tonicity modifying agent is selected from the group consisting of NaCl, KCl, mannitol, dextrose, sucrose, sorbitol, and glycerin.

In some embodiments, the preconditioned MSC secretome composition further comprises mono/di-sodium phosphate, mannitol, and trehalose, and wherein the composition has a pH of about pH 7.4.

In some embodiments, the preconditioned MSC secretome composition further comprises divalent cations.

In some embodiments, the divalent cations are selected from the group consisting of Mg2+, Ca2+, and Zn2+.

In some embodiments, the preconditioned MSC secretome composition further comprises di-sodium phosphate/citric acid, mannitol, and trehalose, and wherein the composition has a pH of about pH 6.4.

In some embodiments, the preconditioned MSC secretome composition further comprises an adhesive agent.

In some embodiments, the adhesive agent is selected from the group consisting of hypromellose, Poloxamer 407, Poloxamer 188, Poloxomer 237, Poloxomer 338, Hypromellose, (HPMC), polycarbophil, polyvinylpyrrolidone (PVP), Polyvinyl alcohol (PVA), polyimide, sodium hyaluronate, gellan gum, poly(lactic acid-co-glycolic acid) (PLGA), polysiloxane, polyimide, carboxymethylcellulose (CMC), or hydroxypropyl methylcellulose (HPMC), hydroxy methyl cellulose, hydroxy ethyl cellulose, sodium carboxy methyl cellulose, fibrin glue, polyethyelene glycol, and GelCORE.

In some embodiments, the preconditioned MSC secretome composition does not comprise one or more components selected from the group consisting of: xenobiotic components; Phenol red; peptides and biomolecules <3 kDa; antibiotics; protein aggregates >200 nm; cells; non-exosome/non-Extracellular Vesicles cell debris; hormones; and L-glutamine.

In some embodiments, the preconditioned MSC secretome composition comprises: HGF; Pentraxin-3 (TSG-14); VEGF; TIMP-1; Serpin E1; and <5 ng/mL IL-8.

In some embodiments, the preconditioned MSC secretome composition comprises:
i. 0.3-4.5 ng/mL HGF;
ii. 0.5-20 ng/mL Pentraxin-3 (TSG-14);
iii. 100-600 pg/mL VEGF;
iv. 10-200 ng/mL TIMP-1;
v. 20-80 ng/mL Serpin E1; and
vi. <5 ng/mL IL-8.

In some embodiments, the preconditioned MSC secretome composition comprise an anti-angiogenic MSC secretome or an anti-scarring MSC secretome.

The present invention also provides a method of treatment for an ocular condition in a subject in need thereof comprising administering to the subject a preconditioned mesenchymal stem cell (MSC) secretome composition, wherein the preconditioned MSC secretome composition is a stable preconditioned mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg hypromellose per mL; and
wherein the pH is about 4.7 to about 7.5.

The present invention also provides a method of treatment for an ocular condition in a subject in need thereof comprising administering to the subject a preconditioned mesenchymal stem cell (MSC) secretome composition, wherein the preconditioned MSC secretome composition is a stable preconditioned mesenchymal stem cell (MSC) secretome formulation comprising:
i. 0.004%-0.08% w/w of MSC secretome
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w hypromellose; and
wherein the pH is about 4.7 to about 7.5.

The present invention also provides a stable preconditioned mesenchymal stem cell (MSC) secretome formulation comprising:
i. 1-20 μg, optionally 2 μg-8 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg optionally hypromellose per mL; and
wherein the pH is about 4.7 to about 7.5.

The present invention also provides a stable preconditioned mesenchymal stem cell (MSC) secretome formulation comprising:
i. 0.004%-0.0375%, optionally 0.008%-0.015% w/w of MSC secretome;
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w optionally hypromellose; and
wherein the pH is about 4.7 to about 7.5.

The present invention also provides a stable preconditioned mesenchymal stem cell (MSC) secretome formulation comprising:
i. 6 μg of MSC secretome per mL;
ii. 2.28 mg monobasic sodium phosphate per mL;
iii. 11.45 mg dibasic sodium phosphate per mL;
iv. 12.2 mg mannitol per mL;
v. 24 mg trehalose dihydrate;
vi. 1 mg optionally hypromellose per mL; and
wherein the pH is about 7.4.

The present invention also provides a stable preconditioned mesenchymal stem cell (MSC) secretome formulation comprising:
i. 6 μg of MSC secretome per mL;
ii. 1.31 mg monobasic sodium phosphate per mL;
iii. 5.73 mg dibasic sodium phosphate per mL;
iv. 12.2 mg mannitol per mL;
v. 24 mg trehalose dihydrate per mL;
vi. 1 mg hypromellose per mL;
vii. 0.2 mg magnesium chloride per mL;
viii. 1.17 mg sodium chloride per mL; and
wherein the pH is about 7.4.

The present invention also provides a stable preconditioned mesenchymal stem cell (MSC) secretome formulation comprising:
i. 0.012% w/w of MSC secretome;
ii. 4.5% w/w monobasic sodium phosphate;
iii. 22.4% w/w dibasic sodium phosphate;
iv. 24% w/w mannitol;
v. 47.1% w/w trehalose dehydrate;
vi. 2.0% w/w optionally hypromellose; and
wherein the pH is about 7.4.

The present invention also provides a stable preconditioned mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg optionally hypromellose per mL; and
wherein the pH is about 4.7 to about 7.5.

The present invention also provides a stable preconditioned mesenchymal stem cell (MSC) secretome formulation comprising:
i. 0.004%-0.08% w/w of MSC secretome;
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w optionally hypromellose; and
wherein the pH is about 4.7 to about 7.5.

In some embodiments of the stable preconditioned mesenchymal stem cell (MSC) secretome formulation the formulation does not comprise hypromellose.

The present invention also provides a method of treatment for an ocular condition in a subject in need thereof comprising administering to the subject a preconditioned mesenchymal stem cell (MSC) secretome composition, wherein the preconditioned MSC secretome composition is a stable preconditioned mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg optionally hypromellose per mL; and
wherein the pH is about 4.7 to about 7.5.

The present invention also provides a method of treatment for an ocular condition in a subject in need thereof comprising administering to the subject a preconditioned mesenchymal stem cell (MSC) secretome composition, wherein the preconditioned MSC secretome composition is a stable preconditioned mesenchymal stem cell (MSC) secretome formulation comprising:
i. 0.004%-0.08% w/w of MSC secretome
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w optionally hypromellose; and
wherein the pH is about 4.7 to about 7.5.

In some embodiments of method of treatment for an ocular condition, the preconditioned MSC secretome composition and/or formulation used for the method of treatment does not comprise hypromellose.

In some embodiments of the methods described herein, the preconditioned MSC secretome composition and/or formulation does not comprise hypromellose.

In some embodiments of the preconditioned MSC secretome composition and/or formulation, the composition and/or formulation does not comprise hypromellose.

ii. MSC Secretome—Processing

The conditioned medium comprising the MSC secretome described herein can in some embodiments be collected and filtered and/or purified to remove cell particulate and/or other detrimental components. For example, as described above under step (v) harvesting the second culture media from step (iv) as conditioned media. The filtration membranes used herein may be selected from any of those known in the art having a suitable membrane and configuration, such that they are capable of retaining the desired MSC secretome components while allowing the cell particulate and/or other detrimental components pass through. Thus, one may employ any suitable membrane which permits the retention of cells under the fluid dynamic conditions selected whilst allowing the detrimental components to pass through for removal. In some embodiments, an upper limit of pore size of about 5 microns and a lower limit of about 0.1 microns would be suitable. In some embodiments, filtration can be performed using a micropore filter. In some embodiments, filtration can be performed using a 0.5 μm to a 0.2 μm filter. In some embodiments, filtration can be performed using a 0.5 μm, 0.45 μm, 0.4 μm, 0.35 μm, 0.3 μm, 0.25 μm, 0.22 μm and/or a 0.2 μm filter. In some embodiments, filtration can be performed using a 0.45 μm filter. In some embodiments, filtration can be performed using a 0.22 μm filter. In some embodiments, filtration/purification can be performed using a low protein binding polyvinylidene difluoride (PVDF) membranes. In some embodiments, filtration/purification can be performed using polyethersulfone (PES).

In some embodiments, the filtering is by ultra-filtration. In some embodiments, the conditioned medium is filtered using a filter size of 3 kD (to achieve purification, desalting, and concentration in the processed conditioned medium of molecules larger than the filter size). In some embodiments, a filter size of less than 3 kD is used to filter the conditioned medium, while in other embodiments a filter size of greater than 3 kD is used, depending on the application for which the processed conditioned medium is used. In other embodiments, ultra-filtration of harvested conditioned medium is carried out using a filter of a different pore size (e.g., 2 kD, <2 kD or >2 kD) selected to determine the size of components of the resulting processed conditioned medium comprising the MSC secretome.

In some embodiments, the detrimental components in the growth supporting media are removed by medium exchange, preferably via “cross-flow filtration”. Cross-flow filtration refers to a mode of filtration where a suspension of MSC secretome cells flows substantially parallel to a filter which is permeable to a component of the suspension other than cells. The cross-flow filtration process is characterized by a set of fluid dynamic parameters including Re=Reynolds number, γw=wall shear rate, ΔP=pressure drop and TMP=transmembrane pressure. Re, γw and ΔP will depend on the geometry of the filtration system, flow conditions and fluid properties. Such cross-flow processes can, in some embodiments, include hollow fiber filtration systems as well. See, for example, U.S. Pat. No. 5,053,334, incorporated herein by reference in its entirety.

In some embodiments, the MSC secretome can be further subject to concentrated in the absence of filtration and/or after filtration. In some embodiments, the MSC secretome can be concentrated using hollow fiber tangential flow technology, or

In some embodiments, the MSC secretome can be concentrated using centrifugation based size exclusion technique, for example, amicons and/or centricons can be employed during the centration step. In some embodiments, the size cutoff is a 3-10 kDa MW cutoff. In some embodiments, the molecular weight cutoff for use during centrifugation based size exclusion technique concentration methods is at least about 3 kDa, at least about 4 kDa, at least about 5 kDa, at least about 6 kDa, at least about 7 kDa, at least about 8 kDa, at least about 9 kDa, or at least about 10 kDa.

In some embodiments, the MSC secretome is concentrated about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, or about 100-fold. In some embodiments, the MSC secretome is concentrated about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, or about 100-fold as compared to the conditioned media prior to concentration

In some embodiments, the MSC secretome is further buffer exchanged after the concentration step into the final formulation buffer. In some embodiments, the MSC secretome is further buffer exchanged after the concentration step into the final formulation buffer without an adhesive agent. In some embodiments, buffer exchange comprises altering the buffer components of the MSC secretome. In some embodiments, the MSC secretome is not diluted during the buffer exchange step. In some embodiments, the MSC secretome is diluted less than 1%, less than 5%, less than 10%, less than 15%, less than 20%, or less than 25% during the buffer exchange step.

In some embodiments, the MSC secretome is buffer exchanged after the concentration step such that the all traces of culture media components are removed. In some embodiments, the MSC secretome is buffer exchanged after the concentration step such that less than about 25%, less than about 20%, less than about 15%, less than about 10%, less than about 5%, less than about 4%, less than about 3%, less than about 2%, or less than about 1% or about 0% of the culture media components remain.

iii. MSC Secretome—Formulating

In some embodiments, the MSC secretome is prepared in a formulation comprising about 2 μg-20 μg per 1 mL of MSC secretome. In some embodiments, the MSC secretome is prepared in a formulation comprising 0.004% to 0.0375% per mL of MSC secretome.

In some embodiments, the MSC secretome is prepared in a formulation comprising about 2 μg-8 μg per 1 mL of MSC secretome. In some embodiments, the MSC secretome is prepared in a formulation comprising 0.008% to 0.015% per mL of MSC secretome.

In some embodiments, the MSC secretome is prepared in a formulation comprising 2 mg-3 mg per mL of monobasic sodium phosphate. In some embodiments, the MSC secretome is prepared in a formulation comprising 4% to 5% per mL of monobasic sodium phosphate.

In some embodiments, the MSC secretome is prepared in a formulation comprising 11 mg-12 mg per mL of dibasic sodium phosphate. In some embodiments, the MSC secretome is prepared in a formulation comprising 21.5% to 23% per mL of dibasic sodium phosphate.

In some embodiments, the MSC secretome is prepared in a formulation comprising 11.5 mg-13 mg per mL of mannitol. In some embodiments, the MSC secretome is prepared in a formulation comprising 23% to 25% per mL of mannitol.

In some embodiments, the MSC secretome is prepared in a formulation comprising 23 mg-25 mg per mL of trehalose dihydrate. In some embodiments, the MSC secretome is prepared in a formulation comprising 46% to 48% per mL of trehalose dihydrate.

In some embodiments, the MSC secretome is prepared in a formulation that does not comprise hypromellose. In some embodiments, the MSC secretome is prepared in a formulation that optionally comprises hypromellose. In some embodiments, the MSC secretome is prepared in a formulation comprising 0.5 mg-2 mg per mL of hypromellose. In some embodiments, the MSC secretome is prepared in a formulation comprising 1% to 3% per mL of hypromellose.

In some embodiments, the MSC secretome is prepared in a formulation comprising hydrochloric acid and/or sodium hydroxide. In some embodiments, the MSC secretome is prepared in a formulation comprising hydrochloric acid. In some embodiments, the MSC secretome is prepared in a formulation comprising sodium hydroxide. In some embodiments, the hydrochloric acid and/or sodium hydroxide is employed to obtain the desired pH.

In some embodiments, the MSC secretome is prepared in a formulation comprising the components as provided in Tables 1-4 below:

TABLE 1a
MSC secretome formulation embodiment.

| | Amount per 1 | | Percent present |
|---|---|---|---|
| Constituent Present | mL of product | | (%) per 1 mL |
| MSC secretome | 0.006 mg (6 μg) | | 0.012 |
| Monobasic sodium phosphate | 2.28 | mg | 4.5 |
| Dibasic sodium phosphate | 11.45 | mg | 22.4 |
| Mannitol | 12.2 | mg | 24.0 |
| Trehalose Dihydrate | 24 | mg | 47.1 |
| Hypromellose | 1 | mg | 2.0 |
| Hydrochloric acid and/or | adjust as required | | adjust as required |
| sodium hydroxide | | | |
| Total quantity | 50.936 | mg | 100% |

TABLE 1b
MSC secretome formulation embodiment.

| | Amount per 1 | | Percent present |
|---|---|---|---|
| Constituent Present | mL of product | | (%) per 1 mL |
| MSC secretome | 0.006 mg (6 μg) | | 0.012 |
| Monobasic sodium phosphate | 1.14 | mg | 2.3 |
| Dibasic sodium phosphate | 5.72 | | 11.2 |
| Mannitol | 12.2 | mg | 24.0 |
| Trehalose Dihydrate | 24 | mg | 47.1 |
| Hypromellose | 1 | mg | 2.0 |
| Hydrochloric acid and/or | adjust as required | | adjust as required |
| sodium hydroxide | | | |

TABLE 1c
MSC secretome formulation embodiment.

| Constituent Present | Amount per 1 mL of product | |
|---|---|---|
| MSC secretome | 0.006 mg (6 μg) | |
| Monobasic sodium phosphate | 1.31 | mg |
| Dibasic sodium phosphate | 5.73 | mg |
| Mannitol | 12.2 | mg |
| Trehalose Dihydrate | 24 | mg |
| Hypromellose | 1 | mg |
| Hydrochloric acid and/or | adjust as required | |
| sodium hydroxide | | |
| Magnesium chloride, | 0.2 | mg |
| hexahydrate | | |
| Sodium chloride | 1.76 | mg |
| Total quantity | 50.936 | mg |

TABLE 2
MSC secretome formulation embodiment.

| Constituent Present | Concentration |
|---|---|
| MSC secretome | 0.006 mg/mL (6 μg/mL) |
| Histidine HCl | 10 mM |
| α,α-trehalose dihydrate | 10% |
| polysorbate 20 | 0.01% |

In some embodiments, the MSC secretome formulation provided in Table 2 has a pH of about 5.5.

TABLE 3
MSC secretome formulation embodiment.

| Constituent Present | Concentration |
|---|---|
| MSC secretome | 0.006 mg/mL (6 μg/mL) |
| sodium phosphate | 10 mM |
| sodium chloride | 40 mM |
| polysorbate 20 | 0.03% |
| sucrose | 5% |

In some embodiments, the MSC secretome formulation provided in Table 3 has a pH of about 6.2.

TABLE 4
MSC secretome formulation embodiment.

| Constituent Present | Concentration |
|---|---|
| MSC secretome | 0.006 mg/mL (6 μg/mL) |
| sodium citrate | 10 mM |
| sucrose | 5.8% |
| polysorbate 20, pH 5.5 | 0.01% |

In some embodiments, the MSC secretome formulation provided in Table 4 has a pH of about 7.2. In some embodiments, the MSC secretome is formulated with Water for Injection in accordance with USP standards.

### D. Assay Methods/Therapeutic Properties

In some embodiments of the invention, the MSC secretome is processed to achieve certain ingredient ratios/concentrations as well as properties for the MSC secretome.

In some embodiments, the MSC secretome composition comprises ratios of anti-angiogenic to pro-angiogenic wherein the ratio is >1. In some embodiments, the MSC secretome composition comprises ratios of anti-angiogenic to pro-angiogenic wherein the ratio is >2, >3, >4, or >5. In some embodiments, the MSC secretome composition comprises an increased concentration of pro-angiogenic factors (relative to the concentration of pro-angiogenic factors in conditioned medium from which the MSC secretome composition is produced). In some embodiments, the MSC secretome composition comprises a sum of several anti-angiogenic factors that exceeds the level of VEGF. In some embodiments, the MSC secretome composition comprises a sum of several anti-angiogenic factors such that the ratio of the more than 1 anti-angiogenic factor to VEGF is >2, >3, >4, or >5. In some embodiments, the MSC secretome composition comprises one or more anti-angiogenic factor, and wherein the sum of the concentration of the one or more anti-angiogenic factors relative to the concentration of VEGF is >2, >3, >4, or >5. In some embodiments, pro-angiogenic factors include but are not limited to Serpin E1 to VEGF-A. In some embodiments, the pro-angiogenic factor is Serpin E1. In some embodiments, the pro-angiogenic factor is VEGF-A.

In some embodiments of the invention, the MSC secretome is processed to achieve certain potency performance criteria. In some embodiments, the buffer exchange step promotes obtaining a potent MSC secretome.

Extracellular Vesicles are membrane bound particles that carry cargo of soluble and insoluble substances mentioned above. The term “Extracellular Vesicles” refers a group of secreted or shedded vesicles of various species. These are generally divided into the following subtypes: 1) microvesicles or Shed microvesicles which typically exhibit a size range of 50-1500 nm; 2) exosomes which typically exhibit a size range of 30-120 nm; and 3) vesicles which typically exhibit a size range of less than 500 nm (i.e., <500 nm). (See, for example, WO2019016799, incorporated by reference herein in its entirety.) In some embodiments, the MSC secretome can be analyzed for particle count and/or to quantitate the extracellular vesicles (EVs) present in the secretome.

In some embodiments, Evs are present in a concentration of about 2.5×10\hat{ }5/uL, 2.6×10\hat{ }5/uL, 2.7×10\hat{ }5/uL, 2.8×10\hat{ }5/uL, 2.9×10\hat{ }5/uL, 3.0×10\hat{ }5/uL, 3.1×10\hat{ }5/uL, 3.2×10\hat{ }5/uL, 3.3×10\hat{ }5/uL, 3.4×10\hat{ }5/uL, 3.5×10\hat{ }5/uL, 3.6×10\hat{ }5/uL, 3.7×10\hat{ }5/uL, 3.8×10\hat{ }5/uL, 3.9×10\hat{ }5/uL, 4.0×10\hat{ }5/uL, 4.1×10\hat{ }5/uL, 4.2×10\hat{ }5/uL, 4.3×10\hat{ }5/uL, 4.4×10\hat{ }5/uL, 4.5×10\hat{ }5/uL, 4.6×10\hat{ }5/uL, 4.7×10\hat{ }5/uL, 4.8×10\hat{ }5/uL, 4.9×10\hat{ }5/uL, or about 5.0×10\hat{ }5/uL. In some embodiments, Evs are present in a concentration of about 3.8×10\hat{ }5/uL+/−0.8×10\hat{ }5.

In some embodiments, Evs are present in a concentration of about 2.5×10\hat{ }5/uL, 2.6×10\hat{ }5/uL, 2.7×10\hat{ }5/uL, 2.8×10\hat{ }5/uL, 2.9×10\hat{ }5/uL, 3.0×10\hat{ }5/uL, 3.1×10\hat{ }5/uL, 3.2×10\hat{ }5/uL, 3.3×10\hat{ }5/uL, 3.4×10\hat{ }5/uL, 3.5×10\hat{ }5/uL, 3.6×10\hat{ }5/uL, 3.7×10\hat{ }5/uL, 3.8×10\hat{ }5/uL, 3.9×10\hat{ }5/uL, 4.0×10\hat{ }5/uL, 4.1×10\hat{ }5/uL, 4.2×10\hat{ }5/uL, 4.3×10\hat{ }5/uL, 4.4×10\hat{ }5/uL, 4.5×10\hat{ }5/uL, 4.6×10\hat{ }5/uL, 4.7×10\hat{ }5/uL, 4.8×10\hat{ }5/uL, 4.9×10\hat{ }5/uL, or about 5.0×10\hat{ }5/uL and average 110-120 nm in diameter. In some embodiments, Evs are present in a concentration of about 2.5×10\hat{ }5/uL, 2.6×10\hat{ }5/uL, 2.7×10\hat{ }5/uL, 2.8×10\hat{ }5/uL, 2.9×10\hat{ }5/uL, 3.0×10\hat{ }5/uL, 3.1×10\hat{ }5/uL, 3.2×10\hat{ }5/uL, 3.3×10\hat{ }5/uL, 3.4×10\hat{ }5/uL, 3.5×10\hat{ }5/uL, 3.6×10\hat{ }5/uL, 3.7×10\hat{ }5/uL, 3.8×10\hat{ }5/uL, 3.9×10\hat{ }5/uL, 4.0×10\hat{ }5/uL, 4.1×10\hat{ }5/uL, 4.2×10\hat{ }5/uL, 4.3×10\hat{ }5/uL, 4.4×10\hat{ }5/uL, 4.5×10\hat{ }5/uL, 4.6×10\hat{ }5/uL, 4.7×10\hat{ }5/uL, 4.8×10\hat{ }5/uL, 4.9×10\hat{ }5/uL, or about 5.0×10\hat{ }5/uL and average 112-116 nm in diameter. In some embodiments, Evs are present in a concentration of about 2.5×10\hat{ }5/uL, 2.6×10\hat{ }5/uL, 2.7×10\hat{ }5/uL, 2.8×10\hat{ }5/uL, 2.9×10\hat{ }5/uL, 3.0×10\hat{ }5/uL, 3.1×10\hat{ }5/uL, 3.2×10\hat{ }5/uL, 3.3×10\hat{ }5/uL, 3.4×10\hat{ }5/uL, 3.5×10\hat{ }5/uL, 3.6×10\hat{ }5/uL, 3.7×10\hat{ }5/uL, 3.8×10\hat{ }5/uL, 3.9×10\hat{ }5/uL, 4.0×10\hat{ }5/uL, 4.1×10\hat{ }5/uL, 4.2×10\hat{ }5/uL, 4.3×10\hat{ }5/uL, 4.4×10\hat{ }5/uL, 4.5×10\hat{ }5/uL, 4.6×10\hat{ }5/uL, 4.7×10\hat{ }5/uL, 4.8×10\hat{ }5/uL, 4.9×10\hat{ }5/uL, or about 5.0×₁₀\hat{ }5/uL and average 114 nm in diameter. In some embodiments, Evs are present in a concentration of about 3.8×10\hat{ }5/uL+/−0.8×10\hat{ }5 and average 114 nm in diameter.

i. MSC Secretome—Therapeutic Properties

The MSC secretome of the present disclosure exhibits a variety of therapeutic properties, including for example, anti-angiogenic properties (blood vessels and/or lymphatic vessels), anti-fibrotic properties, anti-inflammatory properties, properties promoting cell migration and proliferation, mitogenic promoting properties, anti-oxidative stress/damage properties,

In some embodiments, anti-angiogenic (blood vessels and/or lymphatic vessels) properties can be determined by the presence and/or level of one or more factors in the MSC secretome. In some embodiments, the anti-angiogenic factors include but are not limited to one or more of PEDF, sFLT-1, lower levels of VEGF, and/or Serpin E1. In some embodiments, the anti-angiogenic factors include but are not limited to one or more of PEDF, lower levels of VEGF, and/or Serpin E1. In some embodiments, the anti-angiogenic factor is PEDF. In some embodiments, the anti-angiogenic factor is sFLT-1. In some embodiments, the anti-angiogenic factor corresponds to lower levels of VEGF. In some embodiments, the anti-angiogenic factor is Serpin E1.

In some embodiments, pro-angiogenic (blood vessels and/or lymphatic vessels) properties can be determined by the presence and/or level of one or more factors in the MSC secretome. In some embodiments, the pro-angiogenic factors includes one or more factors selected from the group consisting of VEGF, Angiogenin, IGFBP-3, uPA, Angio-1, Angio-2, Endothelin-1. In some embodiments, the pro-angiogenic factor is VEGF. In some embodiments, the pro-angiogenic factor is Angiogenin. In some embodiments, the pro-angiogenic factors is IGFBP-3. In some embodiments, the pro-angiogenic factor is uPA. In some embodiments, the pro-angiogenic factor is Angio-1. In some embodiments, the pro-angiogenic factor is Angio-2. In some embodiments, the pro-angiogenic factor is Endothelin-1.

In some embodiments, the MSC secretome exhibits anti-fibrotic properties. In some embodiments, such anti-fibrotic properties can be assayed for using standard assays. In some embodiments, the present of various factors and/or activities with regard to the MSC secretome are indicative of anti-fibrotic properties. In some embodiments, factors which are indicative of anti-fibrotic properties include but are not limited to FGF7 and/or FGF10. In some embodiments, the factor indicative of anti-fibrotic properties is FGF7. In some embodiments, the factor indicative of anti-fibrotic properties is FGF10. In some embodiments, the factor indicative of anti-fibrotic properties is HGF. In some embodiments, activities indicative of anti-fibrotic properties include, but are not limited to, activation of SMAD, inhibition of TGFβ pathway, inhibition of myofibroblast differentiation, and/or inhibition of excess ECM deposition. In some embodiments, activities indicative of anti-fibrotic properties include activation of SMAD. In some embodiments, activities indicative of anti-fibrotic properties include inhibition of TGFβ pathway. In some embodiments, activities indicative of anti-fibrotic properties include inhibition of myofibroblast differentiation. In some embodiments, activities indicative of anti-fibrotic properties include inhibition of excess ECM deposition.

In some embodiments, the MSC secretome exhibits anti-inflammatory properties. In some embodiments, the MSC secretome inhibits inflammation. In some embodiments, the MSC secretome inhibits inflammation by 5%, 10%, 15%, 20%, 30%, 40%, 50%, 60%, 70%, 80%. 90%, or 100% (e.g., complete reduction in inflammation). In some embodiments, the MSC secretome prevents degranulation of mast cells.

In some embodiments, the MSC secretome promotes cell migration and proliferation, including for example, mitogenic and motogenic activities. In some embodiments, the MSC secretome promotes mitogenic activities. In some embodiments, the MSC secretome promotes motogenic activities. In some embodiments, the MSC secretome comprises FGF7, which provides for the cell migration and proliferation activities of the MSC secretome.

In some embodiments, the MSC secretome comprises FGF7, which provides for the cell migration and proliferation activities of the MSC secretome.

In some embodiments, the MSC secretome comprises HGF, which provides for the cell migration and proliferation activities of the MSC secretome.

In some embodiments, the MSC secretome comprises anti-apoptotic agents, which provides for the cell migration and proliferation activities of the MSC secretome. In some embodiments, the MSC secretome comprises anti-apoptotic agents include but are not limited to FGF-2, HGF and IGF-1, and which provide for the cell migration and proliferation activities of the MSC secretome. In some embodiments, the MSC secretome comprises anti-apoptotic agents selected from the group the consisting of FGF-2, HGF and IGF-1, and which provide for the cell migration and proliferation activities of the MSC secretome.

In some embodiments, the MSC secretome comprises NGF, which provides for the cell migration and proliferation activities of the MSC secretome.

In some embodiments, the MSC secretome provides for anti-oxidative stress and or reduction in cellular damage. In some embodiments, the MSC secretome comprises anti-oxidative stress and reduction in cellular damage factors. In some embodiments, the anti-oxidative stress and reduction in cellular damage factors include but are not limited to SOD-1, SOD-2, SOD-3, HO-1. In some embodiments, the anti-oxidative stress and reduction in cellular damage factor is selected from the group consisting of SOD-1, SOD-2, SOD-3, HO-1.

ii. MSC Secretome—Biophysical/Biochemical Properties

### Biochemical and Biophysical Characterization:

In some embodiments, the present invention provides methods for characterization of the MSC secretome. In some embodiments, the MSC secretome characterization will include: 1) a comprehensive and/or quantitative mapping of the molecular entities in the MSC secretome; 2) measuring the contributions of select factors to biological activity; and 3) measuring biophysical parameters. In some embodiments, in order to determine the properties of the MSC secretome, various potency assays can be performed on the MSC secretome as described herein. In some embodiments, the MSC secretome can be subjected to a comprehensive and/or quantitative mapping of the molecular entities in the MSC secretome; 2) measuring the contributions of select factors to biological activity; and 3) measuring biophysical parameters. In some embodiments, characterization assays include but are not limited to biophysical assays, biochemical assays, and bioassays. In some embodiments, characterization assays can include but are not limited to physical component characterizations, oxidative stress assays, misfolded protein response assays, ER stress assays, safety analysis, stability assays, proliferation assays, migration assays, adhesion assays, neovascularization assays, differentiation/scarring assays, inflammation assays, immune assays, gliosis assays, tissue explant survival and function assays, organoid development or survival/function, epithelial barrier integrity assays, retinal degeneration assays, and/or assays of inherited retinal disease including human and animal retinal explants, neural protection/neurotrophic assays. In some embodiments, characterization assays are selected from the group consisting of physical component characterizations, oxidative stress assays, misfolded protein response assays, ER stress assays, safety analysis, stability assays, proliferation assays, migration assays, adhesion assays, neovascularization assays, differentiation/scarring assays, inflammation assays, immune assays, gliosis assays, tissue explant survival and function assays, organoid development or survival/function, epithelial barrier integrity assays, retinal degeneration assays, and/or assays of inherited retinal disease including human and animal retinal explants, neural protection/neurotrophic assays.

### Physical Component Characterizations:

In some embodiments, the characterization of the MSC secretome comprises a method employing a combination of bioanalytical techniques. In some embodiments, the characterization of the MSC secretome comprises determining the physical components of the MSC secretome. In some embodiments, characterization of the MSC secretome includes employing protein arrays, enzyme-linked immunosorbent assays (ELISAs), mass spectrometry, and immunoblotting. In some embodiments, the MSC secretome characterization can be used to identify the molecules in the MSC secretome. In some embodiments, protein arrays can be employed to identify factors in the MSC secretome. In some embodiments, mass spectrometry can be employed to determine the presence of one or more factors in the MSC secretome. In some embodiments, quantitative techniques can be employed to measure the levels of one or more factors. In some embodiments, quantitative techniques such as ELISA can be employed to measure the levels of each factor.

In some embodiments, the secretome comprises protein factors and extracellular vesicles (EVs). In some embodiments, the MSC secretome comprises trophic factors. In some embodiments, the protein factors of the MSC secretome comprise Pentraxin-3, TIMP-1, Serpin E1, TSP-1, HGF. In some embodiments, the MSC secretome comprises EVs. In some embodiments, the MSC secretome is analyzed for simple lipid content in order to quantitatively measure total lipid. In some embodiments, the EV fraction if the MSC secretome can be evaluated for EV markers. In some embodiments, the EV fraction if the MSC secretome can be evaluated for EV markers, including but not limited to AUX, TSG101, CD63, CD9, and CD8.

In some embodiments, the secretome comprises extracellular vesicles (EVs) in a size range of 30-200 nm and 1×10⁸ to 5×10⁹ EVs per mL.

In some embodiments, depletion studies can be performed to distill the individual contributions of critical factors. In some embodiments, using an antibody-based pulldown method, defined factors can be removed from the MSC secretome. In some embodiments, depletion can be verified by western blot and then evaluated by one or more bioassays, as described herein below. In some embodiments, depletion studies can be performed to evaluate the contributions of the protein fraction and the EV fraction. In some embodiments, TIMP1 and/or Serpin E1 can be depleted. In some embodiments, TIMP1 and/or Serpin E1 can be depleted.

### Oxidative Stress:

In some embodiments, oxidative stress prevention assays can be performed on the MSC secretome. In some embodiments, the MSC secretome prevents corneal epithelium damage. In some embodiments, the MSC secretome reduces the presence of inflammation. In some embodiments, the MSC secretome reduces the presence of inflammation as determined by an increase in the present of anti-inflammation markers. In some embodiments, the MSC secretome reduces the presence of inflammation as determined by an increase in the present of anti-inflammation markers, such as, for example, IL-8.

### Safety Characterization:

In some embodiments, the MSC secretome can be evaluated for blood compatibility and implementing tests for sterility as well as pyrogen and endotoxin levels. In some embodiments, the MSC secretome can be evaluated blood compatibility. In some embodiments, evaluating blood compatibility includes assays for hemolysis and hemagglutination. In some embodiments, the MSC secretome does not exhibit detrimental effects with systemic exposure. In some embodiments, the MSC secretome does not exhibit detrimental effects with systemic exposure, such as with severe ocular burns. In some embodiments, the MSC secretome does not exhibit hemagglutination activity. In some embodiments, the MSC secretome does not induce hemolysis. In some embodiments, the MSC secretome does not induce hemolytic activity.

In some embodiments, the MSC secretome can be sterile such that it can be administered as part of a pharmaceutical formulation. In some embodiments, the MSC secretome can be free or substantially free of endotoxins. In some embodiments, the MSC secretome can be free or substantially free of microorganisms.

### Stability:

In some embodiments, the biophysical characteristics of the MSC secretome can be evaluated and/or determined. In some embodiments, the fluorescence, static light scattering and dynamic light scatting to characterize protein stability metrics. In some embodiments, the following parameters can be measured to further characterize the secretome: thermal melting, thermal aggregation, Delta G, and/or viscosity. In some embodiments, a thermal melting assay is employed to determine MSC secretome stability. In some embodiments, a thermal aggregation assay is employed to determine MSC secretome stability. In some embodiments, delta G is employed as a measure for determining MSC secretome stability. In some embodiments, viscosity is measured as an MSC secretome characteristic. In some embodiments, viscosity is to determine MSC secretome stability

In some embodiments, biophysical metrics can be employed to establish stability parameters for characterizing different MSC secretome formulations.

In some embodiments, the MSC secretome is stable at −20° C., 4° C., and room temperature (20° C.), for at least 7 days. In some embodiments, the MSC secretome is stable −20° C., 4° C., and room temperature (20° C.), for at least 14 days. In some embodiments, the MSC secretome is stable for at least 7 days, at least 1 week, at least 2 weeks, at least 3 weeks, or at least 1 month. In some embodiments, the MSC secretome is stable for at least 7 days, at least 14 days, at least 1 week, at least 2 weeks, at least 3 weeks, at least 1 month, at least 2 months, or at least 3 months at about −20° C. In some embodiments, the MSC secretome is stable for at least 7 days, at least 14 days, at least 1 week, at least 2 weeks, at least 3 weeks, or at least 1 month at about 4° C. In some embodiments, the MSC secretome is stable for at least 7 days, at least 14 days, at least 1 week, at least 2 weeks, at least 3 weeks, or at least 1 month at about 20° C. (or room temperature).

In some embodiments, the MSC secretome is stable for at least 7 days at about −20° C. In some embodiments, the MSC secretome is stable for at least 7 days at about 4° C. In some embodiments, the MSC secretome is stable for at least 7 days at about 20° C. In some embodiments, the MSC secretome is stable for at least 7 days at about 25° C. (room temperature).

In some embodiments, the MSC secretome is stable for at least 14 days at about −20° C. In some embodiments, the MSC secretome is stable for at least 14 days at about 4° C. In some embodiments, the MSC secretome is stable for at least 14 days at about 20° C. (or room temperature). In some embodiments, the MSC secretome is stable for at least 14 days at about 25° C. (room temperature).

### Epithelial Barrier Integrity Assay

The corneal epithelium, more precisely, the apical surface of the epithelium has a major contribution to the overall barrier properties of the cornea and change to the corneal barrier serves as a sensitive factor for biocompatibility analysis. In some embodiments, the biophysical characteristics of the MSC secretome can be evaluated and/or determined such as by an epithelial barrier integrity assay. In some embodiments, the epithelial barrier integrity assay is a transepithelial electrical resistance (TEER). In some embodiments, the transepithelial electrical resistance (TEER) can be assessed to measure overall barrierroperties. In some embodiments, 3D tissues can be transferred into 24-well plates containing 2 mL of TEER buffer and incubated for 10 min. In some embodiments, TEER can be measured using an epithelial volt-ohm meter EVOMO and the EndOhm-12 chamber (World Precision, Sarasota, FL). In some embodiments, at the end of the procedure, tissues can be used for tissue viability assessment using the following formula:

% Barrier integrity=100×[TEER(treated tissue)/TEER(placebo control)]

In some embodiments, TEER can be employed to evaluate the effect on barrier integrity after topical application of the MSC secretome. In some embodiments, TEER can be employed to evaluate the effect on barrier integrity after topical application of the MSC secretome following corneal epithelial damage caused by topical exposure to nitrogen mustard (NM) utilizing the EpiCorneal tissue model (MatTek Corp). In some embodiments, MSC secretome can be applied topically, for example at 6 μg/ml (diluted in Placebo solution), as described in Example 6. In some embodiments, EpiCorneal tissues were cultured in 5 ml medium at standard culture conditions for 24 h.

### Bioassays

In some embodiments, bioassays can be employed to characterize the MSC secretome. In some embodiments, bioassays can be related to corneal wound healing: epithelial cell migration and proliferation, stromal cell differentiation (e.g., scarring); neovascularization, and inflammation. In some embodiments, bioassays can be employed to evaluate the ability of the MSC secretome to mediate corneal wound healing: epithelial cell migration and proliferation, stromal cell differentiation (scarring); neovascularization; and inflammation.

### Migration and Proliferation:

In some embodiments, the MSC secretome can be evaluated for the ability of the MSC secretome to promote proliferation and migration. In some embodiments, the MSC secretome can be evaluated for the ability of the MSC secretome to promote proliferation. In some embodiments, the MSC secretome can be evaluated for the ability of the MSC secretome to promote migration. In some embodiments, the MSC secretome promotes proliferation and/or migration. In some embodiments, the MSC secretome promotes proliferation. In some embodiments, the MSC secretome promotes migration. In some embodiments, the MSC secretome can be evaluated use a transwell migration assay to determine proliferation promoting ability.

In some embodiments, a migration assay can be employed to evaluate for the ability of the MSC secretome to promote migration. In some embodiments, a migration assay can be employed to evaluate for the ability of the MSC secretome to promote migration, wherein the migration assay is an in vitro wound closure assay In some embodiments, the migration assay can include a “scratch assay” (also referred to as a “scratch wound assay”). In some embodiments, the MSC secretome promotes migration and this promotion of migration is determined and/or examined utilizing a “scratch assay”. Generally, a scratch assay method is based on when artificial gap, also referred to as a “scratch”, occurs on a confluent cell monolayer. The “scratch” can be monitored for the cells on the edge of the newly created gap migrating toward the opening to close/cover the “scratch”. See, for example, Liang, C., Park, A. & Guan, J. In vitro scratch assay: a convenient and inexpensive method for analysis of cell migration in vitro. Nat Protoc 2, 329-333 (2007).)

In some embodiments, the migration assay can include a transwell migration assay employing corneal epithelial cells (or other cell surrogate once validation)—(e.g., wound closure) can be performed on the MSC secretome. In some embodiments, a transwell migration assay employing corneal epithelial as a test for wound closure potency of the MSC secretome. In some embodiments, the MSC secretome promotes wound closure as determined using a transwell migration assay.

In some embodiments, in vitro wound closure assays include but are not limited to a “scratch assay” (also referred to as a “scratch wound assay”) or a circular scratch wound method or circular scratch wound assay or circular wound closure assay.

In some embodiments, human corneal epithelial cell proliferation assays can be performed on the MSC secretome. In some embodiments, human corneal epithelial cell proliferation assays are indicative of a test for wound closure properties of the MSC secretome. In some embodiments, the MSC secretome promotes wound closure as determined using a human corneal epithelial cell proliferation assay.

In some embodiments, a circular scratch wound method or circular scratch wound assay or circular wound closure assay can be employed. In some embodiments, the Oris™ Cell Migration Assay platform can be employed (see, also, as described herein in Example 6).

In some embodiments, an endothelial cell tube formation assay can be performed on the MSC secretome. In some embodiments, an endothelial cell tube formation assays can be indicative that the MSC secretome is not pro-angiogenic. In some embodiments, an endothelial cell tube formation assay provides a measure of the angiogenic potential of the MSC secretome. In some embodiments, the MSC secretome exhibits anti-angiogenic properties. In some embodiments, the MSC secretome is anti-angiogenic properties. In some embodiments, an endothelial cell tube formation assay provides the ratio of anti-angiogenesis signals and pro-angiogenesis signals. In some embodiments, an endothelial cell tube formation assay a negative result will confirm the anti:pro ratio is high and will ensure the MSC secretome will not promote neovascularization. In some embodiments, an endothelial cell tube formation assay a negative result will confirm the anti:pro ratio is high and will ensure the MSC secretome will not promote CNV (choroidal neovascularization) or neovascularization in general. In some embodiments, an inhibition of TGFb induced myofibroblast differentiation assay can be performed on the MSC secretome. In some embodiments, an inhibition of TGFb induced myofibroblast differentiation assay can be performed on the MSC secretome to show that the MSC secretome prevents scarring. In some embodiments, the MSC secretome prevents scarring. In some embodiments, the MSC secretome prevents scarring corneal opacity. In some embodiments, the MSC secretome has low angiogenesis induction. In some embodiments, the MSC secretome has reduced angiogenic response. In some embodiments, the MSC secretome has reduced angiogenic capacity. In some embodiments, the MSC secretome impairs and/or reduces the normal formation of blood vessels in presence of media supportive of angiogenesis. In some embodiments, the MSC secretome has reduced angiogenic capacity when the MSC secretome is compared to untreated control. In some embodiments, the MSC secretome has reduced angiogenic capacity as compared to a sample treated to serum containing media. In some embodiments, the MSC secretome attenuates an angiogenic response. In some embodiments, the MSC secretome reduces the angiogenic response induce by serum free media. In some embodiments, a reduction in angiogenic response is induced by the MSC secretome when secretome plus serum containing media (reduced or no angiogenic response) is compared to serum containing media (angiogenic response). In some embodiments, an angiogenic response is indicated by tube formation in a cell based assay. In some embodiments, an angiogenic response is indicated by tube formation in an endothelial cell tube formation assay.

### Differentiation Scarring:

In some embodiments, the MSC secretome can be evaluated for the ability to prevent differentiation and prevent scarring. In some embodiments, the MSC secretome prevents and/or impairs scarring. In some embodiments, the MSC secretome prevents scarring. In some embodiments, the MSC secretome reduces scarring as compared to other standard treatments. In some embodiments, the MSC secretome prevents and/or impairs differentiation. In some embodiments, the MSC secretome prevents and/or impairs myofibroblast differentiation. In some embodiments, the MSC secretome reduces the loss of corneal transparency. In some embodiments, the MSC secretome reduces the loss of corneal transparency by preventing and/or impairing myofibroblast differentiation.

In some embodiments, the MSC secretome can be evaluated for the ability of the MSC secretome to modulate factors involved in differentiation. In some embodiments, the MSC secretome can be evaluated the ability of the MSC secretome to modulate factors involved in differentiation, including but not limited to TGFB2, Collagen I, Collagen III (normally upregulated during differentiation), TFGB3, MMP-2, and MMP-9 (normally downregulated during differentiation. In some embodiments, the MSC secretome modulates factors selected from the group consisting of TGFB2, Collagen I, Collagen III (normally upregulated during differentiation), TFGB3, MMP-2, and MMP-9 (normally downregulated during differentiation. In some embodiments, the MSC secretome induces a decrease in factors upregulated during normal differentiation. In some embodiments, the MSC secretome induces an increase in factors downregulated during normal differentiation. In some embodiments, the MSC secretome induces a decrease in expression of factors such as SMA. In some embodiments, the MSC secretome induces a decrease in expression of factors such as SMA which is indicative of MSC secretome potency.

### Neovascularization:

In some embodiments, the MSC secretome can be evaluated for the ability to prevent neovascularization. In some embodiments, the MSC secretome prevents, impairs, inhibits, and/or reduces neovascularization. In some embodiments, the MSC secretome inhibits or does not promote neovascularization. In some embodiments, the MSC secretome can be evaluated for the ability to prevent angiogenesis. In some embodiments, the MSC secretome prevents, impairs, inhibits, and/or reduces angiogenesis. In some embodiments, the MSC secretome inhibits angiogenesis.

In some embodiments, the MSC secretome can be further evaluated using depletion assays. In some embodiments, the MSC secretome can be depleted of specified factors. In some embodiments, the MSC secretome can be depleted of specified factors, including for example, but not limited to TIMP1 and/or Serpin E1. In some embodiments, the MSC secretome can be depleted of TIMP1 and/or Serpin E1. In some embodiments, the MSC secretome can be depleted of TIMP1. In some embodiments, the MSC secretome can be depleted of Serpin E1.

### Inflammation:

In some embodiments, the MSC secretome can be evaluated for the ability to prevent, impair, inhibit, and/or reduce inflammation. In some embodiments, the MSC secretome prevents, impairs, inhibits, and/or reduces inflammation. In some embodiments, the MSC secretome inhibits inflammation. In some embodiments, the MSC secretome is characterized in vitro and/or in vivo to determine the ability to prevent, impair, inhibit, and/or reduce inflammation. In some embodiments, the MSC secretome prevents, impairs, inhibits, and/or reduces inflammation in vitro and/or in vivo. In some embodiments, the MSC secretome prevents, impairs, inhibits, and/or reduces inflammation in vitro. In some embodiments, the MSC secretome prevents, impairs, inhibits, and/or reduces inflammation or in vivo. In some embodiments, a tissue model can be employed to characterizing preventing, impairing, inhibiting, and/or reducing inflammation in vitro. In some embodiments, a 3D tissue model can be employed to characterizing preventing, impairing, inhibiting, and/or reducing inflammation in vitro. In some embodiments, a nitrogen mustard (NM) gas burn model can be used to evaluate preventing, impairing, inhibiting, and/or reducing inflammation in vitro. In some embodiments, a nitrogen mustard (NM) gas burn model can be used to evaluate preventing, impairing, inhibiting, and/or reducing inflammation in vitro and as a surrogate for in vivo conditions. In some embodiments, the cytokine profile in response to treatment with and/or administration of the MSC secretome can be determined. In some embodiments, the levels of specific cytokines can be determined. In some embodiments, the level of IL-8 can be determined. In some embodiments, the level of IL-8 expression can be reduced in tissues treated with the MSC secretome. In some embodiments, the level of IL-8 expression is reduced in tissues treated with the MSC secretome and this is indicative of preventing, impairing, inhibiting, and/or reducing inflammation.

### Oxidative Stress:

In some embodiments, the cell survival is evaluated under oxidative stress conditions, or induced misfolded protein response, e.g., by tunicamycin treatment, or ER stress induced by thapsigargin and/or Brefeldin A treatment. In some embodiments, the oxidative stressor is selected from the group consisting of: FeCl₃-sodium nitrilotriacetate (Fe-NTA), sodium periodate (NaIO₄), 7-ketocholesterol (7-KC), hydrogen peroxide (H₂O₂), all-trans retinoic acid (ATRA), and tert-butyl hydroperoxide (t-BHP). In some embodiments, the misfolded protein response is induced by tunicamycin treatment. In some embodiments, the ER stress response is induced by thapsigargin and Brefeldin A treatment.

In some embodiments, the positive control is selected from the group consisting of vitamin A, vitamin B3 (e.g., niacin [nicotinic acid] and nicotinamide), vitamin C (ascorbic acid), vitamin E (including tocopherols [e.g., α-tocopherol] and tocotriernols), and vitamin E analogs (e.g., trolox [water-soluble]); carotenoids, including carotenes (e.g., β-carotene), xanthophylls (e.g., lutein, zeaxanthin and meso-zeaxanthin), and carotenoids in saffron (e.g., crocin and crocetin); sulfur-containing antioxidants, including glutathione (GSH), N-acetyl-L-cysteine (NAC), bucillamine, S-nitroso-N-acetyl-L-cysteine (SNAC), S-allyl-L-cysteine (SAC), S-adenosyl-L-methionine (SAM), α-lipoic acid and taurine; scavengers of ROS and radicals, including carnosine, N-acetylcarnosine, curcuminoids (e.g., curcumin, demethoxycurcumin and tetrahydrocurcumin), cysteamine, Ebselen, glutathione, hydroxycinnamic acids and derivatives (e.g., esters and amides) thereof (e.g., caffeic acid, rosmarinic acid and tranilast), melatonin and metabolites thereof, nitrones (e.g., disufenton sodium [NXY-059]), nitroxides (e.g., XJB-5-131), polyphenols (e.g., flavonoids [e.g., apigenin, genistein, luteolin, naringenin and quercetin]), superoxide dismutase mimetics (infra), tirilazad, vitamin C, vitamin E and analogs thereof (e.g., α-tocopherol and trolox), and xanthine derivatives (e.g., pentoxifylline); mitochondrial antioxidants/“vitamins”, including ubiquinone (coenzyme Q, such as CoQ10), ubiquinol (a reduced and more bioavailable form of ubiquinone, such as ubiquinol-10), ubiquinone/ubiquinol analogs (e.g., idebenone and mitoquinone) and derivatives; mitochondria-targeted antioxidants, including DMQ, DMMQ, MitoE, MitoQ, Mito-TEMPO, MitoVitE, and the SkQ class of compounds (e.g., SkQ1, SkQ2, SkQ3, SkQB, SkQR1, SkQT, SkQT1, SkQT1(m), SkQT1(p), SkQTK1, SkQTR1, SkQBerb and SkQPalm); inhibitors of enzymes that produce ROS, including NADPH oxidase (NOX) inhibitors (e.g., apocynin, decursin and decursinol angelate [both inhibit NOX-1, -2 and -4 activity and expression], diphenylene iodonium, and GKT-831 [formerly GKT-137831, a dual NOX1/4 inhibitor]), NADH:ubiquinone oxidoreductase (complex I) inhibitors (e.g., metformin and rotenone), xanthine oxidase inhibitors (e.g., allopurinol, oxypurinol, tisopurine, febuxostat, topiroxostat, myo-inositol, phytic acid, and flavonoids [e.g., kaempferol, myricetin and quercetin]), and myeloperoxidase inhibitors (e.g., azide, 4-aminobenzoic acid hydrazide and PF-06667272, and apoE mimetics such as AEM-28 and AEM-28-14); substances that mimic or increase the activity or production of antioxidant enzymes, including superoxide dismutase (SOD) (e.g., SOD mimetics such as manganese (III)- and zinc (III)-porphyrin complexes (e.g., MnTBAP, MnTMPyP and ZnTBAP), manganese (II) penta-azamacrocyclic complexes (e.g., M40401 and M40403), manganese (III)-salen complexes (e.g., those disclosed in U.S. Pat. No. 7,122,537) and OT-551 (a cyclopropyl ester prodrug of tempol hydroxylamine), and resveratrol and apoA-I mimetics such as 4F (both increase expression)), catalase (e.g., catalase mimetics such as manganese (III)-salen complexes [e.g., those disclosed in U.S. Pat. No. 7,122,537], and zinc [increases activity]), glutathione peroxidase (GPx) (e.g., apomorphine and zinc [both increase activity], and beta-catenin, etoposide and resveratrol [all three increase expression]), glutathione reductase (e.g., 4-tert-butylcatechol and redox cofactors such as flavin adenine dinucleotide [FAD] and NADPH [all three enhance activity]), glutathione S-transferase (GST) (e.g., phenylalkyl isothiocyanate-cysteine conjugates (e.g., S—[N-benzyl(thiocarbamoyl)]-L-cysteine), phenobarbital, rosemary extract and carnosol [all enhance activity]), thioredoxin (Trx) (e.g., geranylgeranylacetone, prostaglandin E1 and sulforaphane [all increase expression]), NADPH-quinone oxidoreductase 1 (NQO1) (e.g., flavones [e.g., β-naphthoflavone (5,6-benzoflavone)] and triterpenoids [e.g., oleanolic acid analogs such as TP-151 (CDDO), TP-155 (CDDO methyl ester), TP-190, TP-218, TP-222, TP-223 (CDDO carboxamide), TP-224 (CDDO monomethylamide), TP-225, TP-226 (CDDO dimethylamide), TP-230, TP-235 (CDDO imidazolide), TP-241, CDDO monoethylamide, CDDO mono(trifluoroethyl)amide, and (+)-TBE-B], all of which increase expression by activating Nrf2), heme oxygenase 1 (HO-1) (e.g., curcuminoids (e.g., curcumin), triterpenoids (e.g., oleanolic acid analogs such as TP-225), and apoA-I mimetics (e.g., 4F), all of which increase expression), and paraoxonase 1 (PON-1) (e.g., apoE mimetics [e.g., AEM-28 and AEM-28-14] and apoA-I mimetics [e.g., 4F], both types increasing activity); activators of transcription factors that upregulate expression of antioxidant enzymes, including activators of nuclear factor (erythroid-derived 2)-like 2 (NFE2L2 or Nrf2) (e.g., bardoxolone methyl, OT-551, fumarates (e.g., dimethyl and monomethyl fumarate), dithiolethiones (e.g., oltipraz), flavones (e.g., β-naphthoflavone), isoflavones (e.g., genistein), sulforaphane, trichostatin A (also upregulates glutathione synthesis), triterpenoids (e.g., oleanolic acid analogs [e.g., TP-225]), and melatonin (increases Nrf2 expression)); other kinds of antioxidants, including anthocyanins, benzenediol abietane diterpenes (e.g., carnosic acid), cyclopentenone prostaglandins (such as 15d-PGJ2, which also upregulate glutathione synthesis), flavonoids (e.g., flavonoids in Ginkgo biloba (e.g., myricetin and quercetin [increases levels of GSH, SOD, catalase, GPx and GST]), prenylflavonoids (e.g., isoxanthohumol), flavones (e.g., apigenin), isoflavones (e.g., genistein), flavanones (e.g., naringenin) and flavanols (e.g., catechin and epigallocatechin-3-gallate)), omega-3 fatty acids and esters thereof (supra), phenylethanoids (e.g., tyrosol and hydroxytyrosol), retinoids (e.g., all-trans retinol [vitamin A]), stilbenoids (e.g., resveratrol), uric acid, apoA-I mimetics (e.g., 4F), apoE mimetics (e.g., AEM-28 and AEM-28-14), and minerals (e.g., selenium and zinc [e.g., zinc monocysteine]); and analogs, derivatives and salts thereof.

In some embodiments, the positive control comprises N-acetylcysteine (NAC) or Ebselen.

In some embodiments, the MSC secretome of the present invention induces in the oxidative stress assay a response that is about 5% to 100% or more, of the response induced by the positive control. In some embodiments, the MSC secretome of the present invention induces in the oxidative stress assay a response that is about 10% of the response induced by the positive control. In some embodiments, the MSC secretome of the present invention induces in the oxidative stress assay a response that is about 15% of the response induced by the positive control. In some embodiments, the MSC secretome of the present invention induces in the oxidative stress assay a response that is about 20% of the response induced by the positive control. In some embodiments, the MSC secretome of the present invention induces in the oxidative stress assay a response that is about 25% of the response induced by the positive control. In some embodiments, the MSC secretome of the present invention induces in the oxidative stress assay a response that is about 30% of the response induced by the positive control. In some embodiments, the MSC secretome of the present invention induces in the oxidative stress assay a response that is about 35% of the response induced by the positive control. In some embodiments, the MSC secretome of the present invention induces in the oxidative stress assay a response that is about 40% of the response induced by the positive control. In some embodiments, the MSC secretome of the present invention induces in the oxidative stress assay a response that is about 45% of the response induced by the positive control. In some embodiments, the MSC secretome of the present invention induces in the oxidative stress assay a response that is about 50% of the response induced by the positive control. In some embodiments, the MSC secretome of the present invention induces in the oxidative stress assay a response that is about 55% of the response induced by the positive control. In some embodiments, the MSC secretome of the present invention induces in the oxidative stress assay a response that is about 60% of the response induced by the positive control. In some embodiments, the MSC secretome of the present invention induces in the oxidative stress assay a response that is about 65% of the response induced by the positive control. In some embodiments, the MSC secretome of the present invention induces in the oxidative stress assay a response that is about 70% of the response induced by the positive control. In some embodiments, the MSC secretome of the present invention induces in the oxidative stress assay a response that is about 75% of the response induced by the positive control. In some embodiments, the MSC secretome of the present invention induces in the oxidative stress assay a response that is about 80% of the response induced by the positive control. In some embodiments, the MSC secretome of the present invention induces in the oxidative stress assay a response that is about 85% of the response induced by the positive control. In some embodiments, the MSC secretome of the present invention induces in the oxidative stress assay a response that is about 90% of the response induced by the positive control. In some embodiments, the MSC secretome of the present invention induces in the oxidative stress assay a response that is about 95% of the response induced by the positive control. In some embodiments, the MSC secretome of the present invention induces in the oxidative stress assay a response that is about 100% of the response induced by the positive control. In some embodiments, the MSC secretome of the present invention induces in the oxidative stress assay a response that is about 1-fold to 100-fold or more f the response induced by the positive control. In some embodiments, the MSC secretome of the present invention induces in the oxidative stress assay a response that is about 1-fold the response induced by the positive control. In some embodiments, the MSC secretome of the present invention induces in the oxidative stress assay a response that is about 2-fold the response induced by the positive control. In some embodiments, the MSC secretome of the present invention induces in the oxidative stress assay a response that is about 3-fold the response induced by the positive control. In some embodiments, the MSC secretome of the present invention induces in the oxidative stress assay a response that is about 4-fold the response induced by the positive control. In some embodiments, the MSC secretome of the present invention induces in the oxidative stress assay a response that is about 5-fold the response induced by the positive control. In some embodiments, the MSC secretome of the present invention induces in the oxidative stress assay a response that is about 6-fold the response induced by the positive control. In some embodiments, the MSC secretome of the present invention induces in the oxidative stress assay a response that is about 7-fold the response induced by the positive control. In some embodiments, the MSC secretome of the present invention induces in the oxidative stress assay a response that is about 8-fold the response induced by the positive control. In some embodiments, the MSC secretome of the present invention induces in the oxidative stress assay a response that is about 9-fold the response induced by the positive control. In some embodiments, the MSC secretome of the present invention induces in the oxidative stress assay a response that is about 10-fold the response induced by the positive control. In some embodiments, the MSC secretome of the present invention induces in the oxidative stress assay a response that is about 20-fold the response induced by the positive control. In some embodiments, the MSC secretome of the present invention induces in the oxidative stress assay a response that is about 30-fold the response induced by the positive control. In some embodiments, the MSC secretome of the present invention induces in the oxidative stress assay a response that is about 40-fold the response induced by the positive control. In some embodiments, the MSC secretome of the present invention induces in the oxidative stress assay a response that is about 50-fold the response induced by the positive control. In some embodiments, the MSC secretome of the present invention induces in the oxidative stress assay a response that is about 60-fold the response induced by the positive control. In some embodiments, the MSC secretome of the present invention induces in the oxidative stress assay a response that is about 70-fold the response induced by the positive control. In some embodiments, the MSC secretome of the present invention induces in the oxidative stress assay a response that is about 80-fold the response induced by the positive control. In some embodiments, the MSC secretome of the present invention induces in the oxidative stress assay a response that is about 90-fold the response induced by the positive control. In some embodiments, the MSC secretome of the present invention induces in the oxidative stress assay a response that is about 100-fold or more the response induced by the positive control.

In some embodiments, the positive control for misfolded protein response assay includes but is not limited to GSK2606414 and KIRA6. See, Mahameed, M. et al. Cell death & disease vol. 10, 4 300. 1 Apr. 2019, incorporated herein by reference in its entirety.

In some embodiments, the MSC secretome of the present invention induces in the misfolded protein response assay a response that is about 5% to 100% or more, of the response induced by the positive control. In some embodiments, the MSC secretome of the present invention induces in the misfolded protein response assay a response that is about 10% of the response induced by the positive control. In some embodiments, the MSC secretome of the present invention induces in the misfolded protein response assay a response that is about 15% of the response induced by the positive control. In some embodiments, the MSC secretome of the present invention induces in the misfolded protein response assay a response that is about 20% of the response induced by the positive control. In some embodiments, the MSC secretome of the present invention induces in the misfolded protein response assay a response that is about 25% of the response induced by the positive control. In some embodiments, the MSC secretome of the present invention induces in the misfolded protein response assay a response that is about 30% of the response induced by the positive control. In some embodiments, the MSC secretome of the present invention induces in the misfolded protein response assay a response that is about 35% of the response induced by the positive control. In some embodiments, the MSC secretome of the present invention induces in the misfolded protein response assay a response that is about 40% of the response induced by the positive control. In some embodiments, the MSC secretome of the present invention induces in the misfolded protein response assay a response that is about 45% of the response induced by the positive control. In some embodiments, the MSC secretome of the present invention induces in the misfolded protein response assay a response that is about 50% of the response induced by the positive control. In some embodiments, the MSC secretome of the present invention induces in the misfolded protein response assay a response that is about 55% of the response induced by the positive control. In some embodiments, the MSC secretome of the present invention induces in the misfolded protein response assay a response that is about 60% of the response induced by the positive control. In some embodiments, the MSC secretome of the present invention induces in the misfolded protein response assay a response that is about 65% of the response induced by the positive control. In some embodiments, the MSC secretome of the present invention induces in the misfolded protein response assay a response that is about 70% of the response induced by the positive control. In some embodiments, the MSC secretome of the present invention induces in the misfolded protein response assay a response that is about 75% of the response induced by the positive control. In some embodiments, the MSC secretome of the present invention induces in the misfolded protein response assay a response that is about 80% of the response induced by the positive control. In some embodiments, the MSC secretome of the present invention induces in the misfolded protein response assay a response that is about 85% of the response induced by the positive control. In some embodiments, the MSC secretome of the present invention induces in the misfolded protein response assay a response that is about 90% of the response induced by the positive control. In some embodiments, the MSC secretome of the present invention induces in the misfolded protein response assay a response that is about 95% of the response induced by the positive control. In some embodiments, the MSC secretome of the present invention induces in the misfolded protein response assay a response that is about 100% of the response induced by the positive control. In some embodiments, the MSC secretome of the present invention induces in the misfolded protein response assay a response that is about 1-fold to 100-fold or more f the response induced by the positive control. In some embodiments, the MSC secretome of the present invention induces in the misfolded protein response assay a response that is about 1-fold the response induced by the positive control. In some embodiments, the MSC secretome of the present invention induces in the misfolded protein response assay a response that is about 2-fold the response induced by the positive control. In some embodiments, the MSC secretome of the present invention induces in the misfolded protein response assay a response that is about 3-fold the response induced by the positive control. In some embodiments, the MSC secretome of the present invention induces in the misfolded protein response assay a response that is about 4-fold the response induced by the positive control. In some embodiments, the MSC secretome of the present invention induces in the misfolded protein response assay a response that is about 5-fold the response induced by the positive control. In some embodiments, the MSC secretome of the present invention induces in the misfolded protein response assay a response that is about 6-fold the response induced by the positive control. In some embodiments, the MSC secretome of the present invention induces in the misfolded protein response assay a response that is about 7-fold the response induced by the positive control. In some embodiments, the MSC secretome of the present invention induces in the misfolded protein response assay a response that is about 8-fold the response induced by the positive control. In some embodiments, the MSC secretome of the present invention induces in the misfolded protein response assay a response that is about 9-fold the response induced by the positive control. In some embodiments, the MSC secretome of the present invention induces in the misfolded protein response assay a response that is about 10-fold the response induced by the positive control. In some embodiments, the MSC secretome of the present invention induces in the misfolded protein response assay a response that is about 20-fold the response induced by the positive control. In some embodiments, the MSC secretome of the present invention induces in the misfolded protein response assay a response that is about 30-fold the response induced by the positive control. In some embodiments, the MSC secretome of the present invention induces in the misfolded protein response assay a response that is about 40-fold the response induced by the positive control. In some embodiments, the MSC secretome of the present invention induces in the misfolded protein response assay a response that is about 50-fold the response induced by the positive control. In some embodiments, the MSC secretome of the present invention induces in the misfolded protein response assay a response that is about 60-fold the response induced by the positive control. In some embodiments, the MSC secretome of the present invention induces in the misfolded protein response assay a response that is about 70-fold the response induced by the positive control. In some embodiments, the MSC secretome of the present invention induces in the misfolded protein response assay a response that is about 80-fold the response induced by the positive control. In some embodiments, the MSC secretome of the present invention induces in the misfolded protein response assay a response that is about 90-fold the response induced by the positive control. In some embodiments, the MSC secretome of the present invention induces in the misfolded protein response assay a response that is about 100-fold or more the response induced by the positive control.

In some embodiments, the positive control for ER stress assay includes but is not limited to 4-phenyl butyric acid. See, Zeng, M. et al. Toxicology letters vol. 271 (2017): 26-37, incorporated herein by reference in its entirety.

In some embodiments, the MSC secretome of the present invention induces in the ER stress assay a response that is about 5% to 100% or more, of the response induced by the positive control. In some embodiments, the MSC secretome of the present invention induces in the ER stress assay a response that is about 10% of the response induced by the positive control. In some embodiments, the MSC secretome of the present invention induces in the ER stress assay a response that is about 15% of the response induced by the positive control. In some embodiments, the MSC secretome of the present invention induces in the ER stress assay a response that is about 20% of the response induced by the positive control. In some embodiments, the MSC secretome of the present invention induces in the ER stress assay a response that is about 25% of the response induced by the positive control. In some embodiments, the MSC secretome of the present invention induces in the ER stress assay a response that is about 30% of the response induced by the positive control. In some embodiments, the MSC secretome of the present invention induces in the ER stress assay a response that is about 35% of the response induced by the positive control. In some embodiments, the MSC secretome of the present invention induces in the ER stress assay a response that is about 40% of the response induced by the positive control. In some embodiments, the MSC secretome of the present invention induces in the ER stress assay a response that is about 45% of the response induced by the positive control. In some embodiments, the MSC secretome of the present invention induces in the ER stress assay a response that is about 50% of the response induced by the positive control. In some embodiments, the MSC secretome of the present invention induces in the ER stress assay a response that is about 55% of the response induced by the positive control. In some embodiments, the MSC secretome of the present invention induces in the ER stress assay a response that is about 60% of the response induced by the positive control. In some embodiments, the MSC secretome of the present invention induces in the ER stress assay a response that is about 65% of the response induced by the positive control. In some embodiments, the MSC secretome of the present invention induces in the ER stress assay a response that is about 70% of the response induced by the positive control. In some embodiments, the MSC secretome of the present invention induces in the ER stress assay a response that is about 75% of the response induced by the positive control. In some embodiments, the MSC secretome of the present invention induces in the ER stress assay a response that is about 80% of the response induced by the positive control. In some embodiments, the MSC secretome of the present invention induces in the ER stress assay a response that is about 85% of the response induced by the positive control. In some embodiments, the MSC secretome of the present invention induces in the ER stress assay a response that is about 90% of the response induced by the positive control. In some embodiments, the MSC secretome of the present invention induces in the ER stress assay a response that is about 95% of the response induced by the positive control. In some embodiments, the MSC secretome of the present invention induces in the ER stress assay a response that is about 100% of the response induced by the positive control. In some embodiments, the MSC secretome of the present invention induces in the ER stress assay a response that is about 1-fold to 100-fold or more f the response induced by the positive control. In some embodiments, the MSC secretome of the present invention induces in the ER stress assay a response that is about 1-fold the response induced by the positive control. In some embodiments, the MSC secretome of the present invention induces in the ER stress assay a response that is about 2-fold the response induced by the positive control. In some embodiments, the MSC secretome of the present invention induces in the ER stress assay a response that is about 3-fold the response induced by the positive control. In some embodiments, the MSC secretome of the present invention induces in the ER stress assay a response that is about 4-fold the response induced by the positive control. In some embodiments, the MSC secretome of the present invention induces in the ER stress assay a response that is about 5-fold the response induced by the positive control. In some embodiments, the MSC secretome of the present invention induces in the ER stress assay a response that is about 6-fold the response induced by the positive control. In some embodiments, the MSC secretome of the present invention induces in the ER stress assay a response that is about 7-fold the response induced by the positive control. In some embodiments, the MSC secretome of the present invention induces in the ER stress assay a response that is about 8-fold the response induced by the positive control. In some embodiments, the MSC secretome of the present invention induces in the ER stress assay a response that is about 9-fold the response induced by the positive control. In some embodiments, the MSC secretome of the present invention induces in the ER stress assay a response that is about 10-fold the response induced by the positive control. In some embodiments, the MSC secretome of the present invention induces in the ER stress assay a response that is about 20-fold the response induced by the positive control. In some embodiments, the MSC secretome of the present invention induces in the ER stress assay a response that is about 30-fold the response induced by the positive control. In some embodiments, the MSC secretome of the present invention induces in the ER stress assay a response that is about 40-fold the response induced by the positive control. In some embodiments, the MSC secretome of the present invention induces in the ER stress assay a response that is about 50-fold the response induced by the positive control. In some embodiments, the MSC secretome of the present invention induces in the ER stress assay a response that is about 60-fold the response induced by the positive control. In some embodiments, the MSC secretome of the present invention induces in the ER stress assay a response that is about 70-fold the response induced by the positive control. In some embodiments, the MSC secretome of the present invention induces in the ER stress assay a response that is about 80-fold the response induced by the positive control. In some embodiments, the MSC secretome of the present invention induces in the ER stress assay a response that is about 90-fold the response induced by the positive control. In some embodiments, the MSC secretome of the present invention induces in the ER stress assay a response that is about 100-fold or more the response induced by the positive control.

In some embodiments, the cells used to evaluate cell survival under oxidative stress, misfolded protein response, or ER stress response are selected from the group consisting of ARPE-19, primary human RPE, 661 W, and MIO-M1 human Müller cell lines.

In some embodiments, the cells are seeded in 96-well plates at 30% confluency and allowed to adhere overnight, before loading said cells with chloromethyl derivative of H2DCFDA (CM-H2DCFDA) and Mitosoxred for 30 minutes. In some embodiments, the cells are washed prior to treatment with various concentrations of NaIO₄, H₂O₂, 7-KC, ATRA, or t-BHP in the absence/presence of NAC for 1 hour. In some embodiments, the relative levels of reactive oxygen species (ROS) production are measured in parallel to cell viability after 1 hour, 2 hours, 3 hours, 6 hours, 12 hours, 24 hours, or more, post treatment. In some embodiments, the level of ROS production is increased by 10 to 1000 fold by the oxidative stressor. In some embodiments, the level of ROS production is increased by at least 10-fold by the oxidative stressor. In some embodiments, the level of ROS production is increased by at least 100-fold by the oxidative stressor. In some embodiments, the level of ROS production is increased by at least 1000-fold by the oxidative stressor. In some embodiments, the dose-dependent elevation in ROS is evaluated using CM-H2DCFDA and MitoSOX Red, then correlated with cell survival 24 hours post insult. In some embodiments, the dose-dependent elevation in ROS is compared to that inhibited by NAC (anti-oxidant positive control).

In some embodiments, an MSC secretome provides a protective benefit.

In some embodiments, the benefits of the secretome treatment are evaluated based on the results and findings described herein. In some embodiments, the potency or activity of the secretome is evaluated based on the results and findings described herein. In some embodiments, one, two, or three conditions of oxidative stress are selected to test secretome activity. In most embodiments, the positive control is NAC. In some embodiments, initial studies are run in parallel to monitor ROS levels achieved in the study. In some embodiments, the endpoint is cell survival my Microtiter Tetrazolium (MTT) assay. In some embodiments, cells exposed to selected oxidative insults are treated with various concentrations of secretome, and their survival is evaluated at 24 h post insult. In other embodiments, cells are treated with various concentrations of secretome prior to being exposed to selected oxidative insult, and their survival at 24 h post insult is evaluated. In some embodiments, the negative controls consist of a basal medium containing 1% serum condition, and a heat-denatured secretome condition; the positive control is NAC. In some embodiments, the impact of secretome on cellular ROS levels is evaluated under protective conditions.

### Oxidative Stress Induction Agents

In some embodiments, one or more oxidative stress inducing agents are administered to assayed cells to induce oxidative stress and/or cellular ROS production and the extent of the oxidative stress and/or ROS production in the assayed cells is evaluated.

In some embodiments, the assayed cells are RPE cells including but not limited to ARPE-19, primary human RPE, 661W cells, and MIO-M1 human Müller cells, ocular explant tissue including but not limited to retina, cornea, iris, trabecular meshwork, and/or ciliary body.

In some embodiments, the oxidative stress inducing agents include but are not limited to ketocholesterol, FeCl₃-sodium nitrilotriacetate (Fe-NTA), H₂O₂, T-BHP, all-trans retinal, NaIO4, hydroquinone, and Oxidized Cholysterol (OxLDL).

In some embodiments, the oxidative stress is induced by culturing the cells under hypoxic/anoxic conditions.

In some embodiments, inducing oxidative stress in assayed cell comprises treating the cells with one or more inducing agents for about 0.5 to 96 hours. In some embodiments, inducing oxidative stress in assayed cell comprises treating the cells with one or more inducing agents for about 0.5 to 72 hours. In some embodiments, inducing oxidative stress in assayed cell comprises treating the cells with one or more inducing agents for about 0.5 to 48 hours. In some embodiments, inducing oxidative stress in assayed cell comprises treating the cells with one or more inducing agents for about 0.5 to 24 hours. In some embodiments, inducing oxidative stress in assayed cell comprises treating the cells with one or more inducing agents for about 0.5 to 12 hours. In some embodiments, inducing oxidative stress in assayed cell comprises treating the cells with one or more inducing agents for about 0.5 to 6 hours. In some embodiments, inducing oxidative stress in assayed cell comprises treating the cells with one or more inducing agents for about 6 to 96 hours. In some embodiments, inducing oxidative stress in assayed cell comprises treating the cells with one or more inducing agents for about 12 to 96 hours. In some embodiments, inducing oxidative stress in assayed cell comprises treating the cells with one or more inducing agents for about 24 to 96 hours. In some embodiments, inducing oxidative stress in assayed cell comprises treating the cells with one or more inducing agents for about 36 to 96 hours. In some embodiments, inducing oxidative stress in assayed cell comprises treating the cells with one or more inducing agents for about 48 to 96 hours. In some embodiments, inducing oxidative stress in assayed cell comprises treating the cells with one or more inducing agents for about 60 to 96 hours. In some embodiments, inducing oxidative stress in assayed cell comprises treating the cells with one or more inducing agents for about 72 to 96 hours. In some embodiments, inducing oxidative stress in assayed cell comprises treating the cells with one or more inducing agents for about 84 to 96 hours.

In some embodiments, inducing oxidative stress in assayed cell comprises treating the cells with one or more inducing agents for about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 24, 36, 48, 60, 72, 84, 96 hours or longer.

In some embodiments, inducing oxidative stress in assayed cell comprises culturing the cells under hypoxic/anoxic conditions for about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, 24, 36, 48, 60, 72, 84, 96 hours or longer.

In some embodiments, one or more oxidative stress inducing agents are administered to the assayed cells at a concentration of about 0.01 μM to about 100 mM. In some embodiments, one or more oxidative stress inducing agents are administered to the assayed cells at a concentration of about 0.01 μM to about 0.1 uM. In some embodiments, one or more oxidative stress inducing agents are administered to the assayed cells at a concentration of about 0.1 μM to about 1 μM. In some embodiments, one or more oxidative stress inducing agents are administered to the assayed cells at a concentration of about 1 μM to about 10 μM. In some embodiments, one or more oxidative stress inducing agents are administered to the assayed cells at a concentration of about 10 μM to about 100 μM. In some embodiments, one or more oxidative stress inducing agents are administered to the assayed cells at a concentration of about 100 μM to about 1 mM. In some embodiments, one or more oxidative stress inducing agents are administered to the assayed cells at a concentration of about 1 mM to about 10 mM. In some embodiments, one or more oxidative stress inducing agents are administered to the assayed cells at a concentration of about 10 mM to about 100 mM.

In some embodiments, the oxidative stress inducing agent is ketocholesterol nitrilotriacetate (Fe-NTA) administered at a concentration from 0.1 to 100 mM for 1-72 hours. In some embodiments, the oxidative stress is induced by administering to the assayed cells Fe-NTA at a concentration of about 0.1 to 1 mM, about 1 to 10 mM, or about 10 to 100 mM, for about 1 to 24 hours, about 24 to 48 hours, or about 48 to 72 hours.

In some embodiments, the oxidative stress inducing agent is FeCl₃-sodium nitrilotriacetate (Fe-NTA) administered at a concentration from 0.1 to 100 mM for 1-72 hours. In some embodiments, the oxidative stress is induced by administering to the assayed cells Fe-NTA at a concentration of about 0.1 to 1 mM, about 1 to 10 mM, or about 10 to 100 mM, for about 1 to 24 hours, about 24 to 48 hours, or about 48 to 72 hours.

In some embodiments, the oxidative stress inducing agent is H₂O₂ administered at a concentration from 0.01 to 1000 μM with exposure between 0.5 to 24 hours. In some embodiments, the oxidative stress is induced by administering to the assayed cells H₂O₂ at a concentration of about 0.01 to 0.1 μM, about 0.1 to 1 μM, about 1 to 10 μM, about 10 to 100 μM, or about 100 to 1000 μM, for about 0.5 to 6 hours, about 6 to 12 hours, about 12 to 18 hours, or about 18 to 24 hours.

In some embodiments, the oxidative stress inducing agent is T-BHP at a concentration from 0.01 to 1000 μM with exposure between 0.5 to 24 hours. In some embodiments, the oxidative stress is induced by administering to the assayed cells t-BHB at a concentration of about 0.01 to 0.1 μM, about 0.1 to 1 μM, about 1 to 10 μM, about 10 to 100 μM, or about 100 to 1000 μM, for about 0.5 to 6 hours, about 6 to 12 hours, about 12 to 18 hours, or about 18 to 24 hours.

In some embodiments, the oxidative stress inducing agent is all-trans retinal, administered at a concentration from 0.01 to 1000 μM with exposure between 0.5 to 24 hours. In some embodiments, the oxidative stress is induced by administering to the assayed cells all-trans retinal at a concentration of about 0.01 to 0.1 μM, about 0.1 to 1 μM, about 1 to 10 μM, about 10 to 100 μM, or about 100 to 1000 μM, for about 0.5 to 6 hours, about 6 to 12 hours, about 12 to 18 hours, or about 18 to 24 hours.

In some embodiments, the oxidative stress inducing agent is NaIO₄, administered at a concentration from 0.01 to 1000 μM with exposure between 0.5 to 24 hours. In some embodiments, the oxidative stress is induced by administering to the assayed cells NaIO₄ at a concentration of about 0.01 to 0.1 μM, about 0.1 to 1 μM, about 1 to 10 μM, about 10 to 100 μM, or about 100 to 1000 μM, for about 0.5 to 6 hours, about 6 to 12 hours, about 12 to 18 hours, or about 18 to 24 hours.

In some embodiments, the oxidative stress inducing agent is Oxidized Cholysterol/OxLDL at a concentration from 10 to 500 ug/ml for 0.5 to 24 hours. In some embodiments, the oxidative stress inducing agent is glucose oxidase (GOx) at a concentration of 1-100 mU/ml for about 24 hours. In some embodiments, the oxidative stress is induced by administering to the assayed cells OxLDL at a concentration of about 10 to 100 pg/mL, about 10 to 100 pg/mL, or about 100 to 500 pg/mL, for about 0.5 to 6 hours, about 6 to 12 hours, about 12 to 18 hours, or about 18 to 24 hours.

### Evaluation of Extent of Oxidative Stress

In some embodiments, the oxidative stress and/or ROS production is evaluated based on measurement of DNA oxidation with anti-8-oxo-2′-deoxyguanosine (8-oxo-dG).

In some embodiments, the oxidative stress and/or ROS production is evaluated based on measurement of lipid oxidation with hiobarbituric acid reactive substances, TBARS).

In some embodiments, the oxidative stress and/or ROS production is evaluated based on measurement of cytoplasmic ROS with CM-H₂ DCFDA.

In some embodiments, the oxidative stress and/or ROS production is evaluated based on measurement of Mitochondrial ROS with Mitoxed.

In some embodiments, the oxidative stress and/or ROS production is evaluated based on measurement of mitochondrial potential (including for example, but not limited to, evaluating JC-1).

In some embodiments, the oxidative stress is induced by one or more of 7-ketocholesterol, FeCl₃-sodium nitrilotriacetate (Fe-NTA), H₂O₂, tert-butyl hydroperoxide, (t-BHP), all-trans retinal, NaIO4, hydroquinone, Oxidized Cholysterol (OxLDL), and the extent of the induced oxidative stress and/or ROS production is evaluated based on one or more of DNA oxidation (anti-8-oxo-2′-deoxyguanosine (8-oxo-dG), lipid oxidation (hiobarbituric acid reactive substances, TBARS), cytoplasmic ROS (CM-H₂ DCFDA), Mitochondrial (ROS) (Mitoxed), and/or mitochondrial potential (JC-1).

In some embodiments, the oxidative stress is induced by ketocholesterol and the extent of the induced oxidative stress and/or ROS production is evaluated based on DNA oxidation. In some embodiments, the oxidative stress is induced by ketocholesterol and the extent of the induced oxidative stress and/or ROS production is evaluated based on lipid oxidation. In some embodiments, the oxidative stress is induced by ketocholesterol and the extent of the induced oxidative stress and/or ROS production is evaluated based on cytoplasmic ROS. In some embodiments, the oxidative stress is induced by ketocholesterol and the extent of the induced oxidative stress and/or ROS production is evaluated based on Mitochondrial (ROS). In some embodiments, the oxidative stress is induced by ketocholesterol and the extent of the induced oxidative stress and/or ROS production is evaluated based on mitochondrial potential.

In some embodiments, the oxidative stress is induced by Fe-NTA and the extent of the induced oxidative stress and/or ROS production is evaluated based on DNA oxidation. In some embodiments, the oxidative stress is induced by Fe-NTA and the extent of the induced oxidative stress and/or ROS production is evaluated based on lipid oxidation. In some embodiments, the oxidative stress is induced by Fe-NTA and the extent of the induced oxidative stress and/or ROS production is evaluated based on cytoplasmic ROS. In some embodiments, the oxidative stress is induced by Fe-NTA and the extent of the induced oxidative stress and/or ROS production is evaluated based on Mitochondrial (ROS). In some embodiments, the oxidative stress is induced by Fe-NTA and the extent of the induced oxidative stress and/or ROS production is evaluated based on mitochondrial potential.

In some embodiments, the oxidative stress is induced by H₂O₂ and the extent of the induced oxidative stress is evaluated based on DNA oxidation. In some embodiments, the oxidative stress is induced by H₂O₂ and the extent of the induced oxidative stress and/or ROS production is evaluated based on lipid oxidation. In some embodiments, the oxidative stress is induced by H₂O₂ and the extent of the induced oxidative stress and/or ROS production is evaluated based on cytoplasmic ROS. In some embodiments, the oxidative stress is induced by H₂O₂ and the extent of the induced oxidative stress and/or ROS production is evaluated based on Mitochondrial (ROS). In some embodiments, the oxidative stress is induced by H₂O₂ and the extent of the induced oxidative stress and/or ROS production is evaluated based on mitochondrial potential.

In some embodiments, the oxidative stress is induced by T-BHP and the extent of the induced oxidative stress and/or ROS production is evaluated based on DNA oxidation. In some embodiments, the oxidative stress is induced by T-BHP and the extent of the induced oxidative stress and/or ROS production is evaluated based on lipid oxidation. In some embodiments, the oxidative stress is induced by T-BHP and the extent of the induced oxidative stress and/or ROS production is evaluated based on cytoplasmic ROS. In some embodiments, the oxidative stress is induced by T-BHP and the extent of the induced oxidative stress and/or ROS production is evaluated based on Mitochondrial (ROS). In some embodiments, the oxidative stress is induced by T-BHP and the extent of the induced oxidative stress and/or ROS production is evaluated based on mitochondrial potential.

In some embodiments, the oxidative stress is induced by all-trans retinal and the extent of the induced oxidative stress and/or ROS production is evaluated based on DNA oxidation. In some embodiments, the oxidative stress is induced by all-trans retinal and the extent of the induced oxidative stress and/or ROS production is evaluated based on lipid oxidation. In some embodiments, the oxidative stress is induced by all-trans retinal and the extent of the induced oxidative stress and/or ROS production is evaluated based on cytoplasmic ROS. In some embodiments, the oxidative stress is induced by all-trans retinal and the extent of the induced oxidative stress and/or ROS production is evaluated based on Mitochondrial (ROS). In some embodiments, the oxidative stress is induced by all-trans retinal and the extent of the induced oxidative stress and/or ROS production is evaluated based on mitochondrial potential.

In some embodiments, the oxidative stress is induced by NaIO₄ and the extent of the induced oxidative stress and/or ROS production is evaluated based on DNA oxidation. In some embodiments, the oxidative stress is induced by NaIO₄ and the extent of the induced oxidative stress and/or ROS production is evaluated based on lipid oxidation. In some embodiments, the oxidative stress is induced by NaIO₄ and the extent of the induced oxidative stress and/or ROS production is evaluated based on cytoplasmic ROS. In some embodiments, the oxidative stress is induced by NaIO₄ and the extent of the induced oxidative stress and/or ROS production is evaluated based on Mitochondrial (ROS). In some embodiments, the oxidative stress is induced by NaIO₄ and the extent of the induced oxidative stress and/or ROS production is evaluated based on mitochondrial potential.

In some embodiments, the oxidative stress is induced by OxLDL and the extent of the induced oxidative stress and/or ROS production is evaluated based on DNA oxidation. In some embodiments, the oxidative stress is induced by OxLDL and the extent of the induced oxidative stress and/or ROS production is evaluated based on lipid oxidation. In some embodiments, the oxidative stress is induced by OxLDL and the extent of the induced oxidative stress and/or ROS production is evaluated based on cytoplasmic ROS. In some embodiments, the oxidative stress is induced by OxLDL and the extent of the induced oxidative stress and/or ROS production is evaluated based on Mitochondrial (ROS). In some embodiments, the oxidative stress is induced by OxLDL and the extent of the induced oxidative stress and/or ROS production is evaluated based on mitochondrial potential.

In some embodiments, the assayed cells is subject to one or more characterization assays including but not limited to physical component characterizations, oxidative stress assays, misfolded protein response assays, ER stress assays, cellular response assays, safety analyses, stability assays, proliferation assay, migration assay, cell survival assay, cell viability assay, MTT assay, morphometry assay (e.g., cell rounding and/or shrinking post-oxidative stress), wound healing assay, neovascularization assays, differentiation/scarring assays, inflammation assays, tissue explant survival and function assays, organoid development or survival/function assays, epithelial barrier integrity assays, retinal degeneration assays, and/or assays of inherited retinal disease including human and animal retinal explants, neural protection/neurotrophic assays.

### Assay Endpoints

In some embodiments, the assayed cells are further evaluated for one or more of phenotypes:
Alteration in epithelial polarization (for example, Na/K ATPase polarity)
Tight junctions (for example, ZO-1, trans-epithelial polarity, and trans-epithelial electrical resistance (TEER))
Phagocytosis of outer segments
Degradation of outer segments
Phagosome composition and function
Cathepsin D distribution activity
Mitochondrial potential
Inflammasome activation/activity
HIf-1 alpha activation/activity
Apoptosis
Ferroptosis
NRF-2 activation/activity
NOQ1 activation/activity
GPX4 activation/activity
SLC7AII activation/activity
Annexin V reactivity
Propidium Iodine staining
IRE1/XBP1 activation/activity
PERK/ATF4/CHOP activation/activity
UPR activation/activity
ACS24 activation/activity
Complement activation/susceptibility
Lipid deposition
Lipid/protein oxidation
Necrosis

### E. Methods of Treatment

The present disclosure also provides methods of treatment using the MSC secretome of the present disclosure. In particular, the MSC secretome finds use in the treatment of ocular conditions. In particular, the MSC secretome finds use in the treatment of ocular conditions, including but not limited to ocular diseases. In some embodiments, the ocular disease is associated with the ocular surface. In some embodiments, the ocular disease is associated with damaged ocular tissue and/or damaged ocular tissue indications. In some embodiments, the MSC secretome finds use in the treatment of ocular conditions, including accelerating wound healing. In some embodiments, the MSC secretome finds use in the treatment of ocular conditions, including reducing scarring. In some embodiments, the MSC secretome finds use in the treatment of ocular conditions, including reducing inflammation. In some embodiments, the MSC secretome finds use in the treatment of ocular conditions, including reducing inflammation and thus promoting growth. In some embodiments, the MSC secretome finds use in treating ocular conditions such as reducing inflammation at the ocular surface. In some embodiments, the MSC secretome finds use in the treatment of ocular conditions, including reducing neovascularization. In some embodiments, the MSC secretome finds use in the treatment of ocular conditions, including reducing neovascularization in the cornea. In some embodiments, the MSC secretome finds use in the treatment of ocular conditions, including dry eye treatment (including, for example, treatment of severe dry eye, including where the epithelial cells are damaged). In some embodiments, the MSC secretome finds use in the treatment of ocular conditions, such as restoring the integrity to damaged ocular tissue. In some embodiments, the MSC secretome finds use in the treatment of ocular conditions, such as accelerating the healing of damaged ocular tissue. In some embodiments, the MSC secretome finds use in the treatment of ocular conditions, such as treating a retinal condition. In some embodiments, the MSC secretome finds use in the treatment of ocular conditions, such as treating a macular disease. In some embodiments, the MSC secretome finds use in the treatment of ocular conditions, such as regenerating damaged ocular nerve tissue. In some embodiments, the MSC secretome finds use in the treatment of ocular conditions, such as regenerating damaged ocular nerve tissue associated with PCED. In some embodiments, the MSC secretome finds use in the treatment of ocular conditions, such as PCED. In some embodiments, the MSC secretome finds use in the treatment of ocular conditions, such as inflammatory damage to the eye surface. In some embodiments, the MSC secretome finds use in the treatment of ocular conditions, such as for example GvHD and/or Sjogren's syndrome. In some embodiments, the MSC secretome finds use in the treatment of ocular conditions, such as limbal stem cell deficiency (LSCD).

In some embodiments, the MSC secretome finds use in accelerating wound healing. In some embodiments, the MSC secretome finds use in reducing scarring. In some embodiments, the MSC secretome finds use in reducing inflammation. In some embodiments, the MSC secretome finds use in reducing inflammation and thus promoting growth. In some embodiments, the MSC secretome finds use in reducing inflammation at the ocular surface. In some embodiments, the MSC secretome finds use in reducing neovascularization. In some embodiments, the MSC secretome finds use in reducing neovascularization in the cornea. In some embodiments, the MSC secretome finds use in the protection and repair of retinal epithelial cells and retinal ganglion cells. In some embodiments, the MSC secretome finds use in induction of trabecular meshwork regeneration and reduction of intraocular pressure.

In some embodiments, the mesenchymal stem cell secretome is administered for the treatment of an ocular disease. In some embodiments, treatment comprises administering to a patient in need thereof therapeutically effective amount of a mesenchymal stem cell secretome composition as described herein to a patient in need thereof. In some embodiments, the mesenchymal stem cell secretome is administered to a patient in need thereof in order to promote or induce ocular wound healing. In some embodiments, the mesenchymal stem cell secretome is administered to a patient in need thereof in order to reduce and/or inhibit neovascularization, reduce and/or inhibit scarring, promote and/or preserve vision, and/or increasing wound closure rate (e.g., decreasing wound closure time). In some embodiments, the mesenchymal stem cell secretome is administered to a patient in need thereof in order to prevent, reduce, and/or inhibit neovascularization. In some embodiments, the mesenchymal stem cell secretome is administered to a patient in need thereof in order to prevent, reduce, and/or inhibit reducing scarring. In some embodiments, the mesenchymal stem cell secretome is administered to a patient in need thereof in order to promote and/or preserve vision. In some embodiments, the mesenchymal stem cell secretome is administered to promote and/or induce closing wound faster wound closure (e.g., reduce the amount of time required for wound closure). In some embodiments, the mesenchymal stem cell secretome prevents, reduces, and/or inhibits or does not promote neovascularization and reducing scarring in order to promote vision preservation. In some embodiments, the mesenchymal stem cell secretome is administered to a patient in need thereof in order to prevent, reduce, and/or inhibit neovascularization and reducing scarring in order to promote vision preservation. In some embodiments, the mesenchymal stem cell secretome prevents, reduces, and/or inhibits inflammation. In some embodiments, the mesenchymal stem cell secretome is administered to a patient in need thereof in order to prevent, reduce, and/or inhibit inflammation.

In some embodiments, the mesenchymal stem cell secretome is administered for the treatment of a visual dysfunction following traumatic injury to ocular structures. In some embodiments, treatment comprises administering to a patient in need thereof a therapeutically effective amount of a mesenchymal stem cell secretome composition as described herein

In some embodiments, the mesenchymal stem cell secretome is administered for the treatment of a traumatic injury of the optic nerve degeneration following concussive injury. In some embodiments, the concussive injury to the eye is selected from the group consisting of ocular contusion and blunt injury to the eye. In some embodiments, the mesenchymal stem cell secretome is administered for the treatment of a traumatic injury of the optic nerve. In some embodiments, treatment comprises administering to a patient in need thereof a therapeutically effective amount of a mesenchymal stem cell secretome composition as described herein.

In some embodiments, the mesenchymal stem cell secretome is administered for ameliorating optic nerve degeneration following concussive injury to the eye. In some embodiments the method for ameliorating optic nerve degeneration comprises administering to the patient a therapeutically effective amount of a mesenchymal stem cell secretome composition as described herein. In some embodiments, the concussive injury to the eye is selected from the group consisting of ocular contusion and blunt injury to the eye. In some embodiments, the concussive injury to the eye an ocular contusion. In some embodiments, the concussive injury to the eye a blunt injury to the eye.

Efficacy readouts can include a reduced in symptoms and/or decreased disease state, including for example, increased quality of life. In some embodiments, reduced in symptoms and/or decreased disease state by 10%, 20%, 30%, 40%, 50%, 60%, 70%, 80%, 90%, 95%, or 100% is indicative of therapeutic efficacy. In some embodiments, reduction in inflammation by 10%, 20%, 30%, 40%, 50%, 60%, 70%, 80%, 90%, 95%, or 100% is indicative of therapeutic efficacy. In some embodiments, a reduction in scarring by 10%, 20%, 30%, 40%, 50%, 60%, 70%, 80%, 90%, 95%, or 100% is indicative of therapeutic efficacy. In some embodiments, a reduction in neovascularization by 10%, 20%, 30%, 40%, 50%, 60%, 70%, 80%, 90%, 95%, or 100% is indicative of therapeutic efficacy.

In some embodiments, the disease or conditions an ocular disease or ocular condition. In some embodiments, the disease or condition is a visual dysfunction following traumatic injury to ocular structures. In some embodiments, the disease or condition is a concussive (e.g., blunt or non-blunt) injury to the eye. In some embodiments, the disease or condition is a burn, including a chemical burn to the eye.

In some embodiments, the mesenchymal stem cell secretome is administered to a particular targeted area. In some embodiments, the particular targeted area is the eye. In some embodiments, the mesenchymal stem cell secretome is administered to a particular targeted area and is formulated so as not to spread to other surrounding areas.

In some embodiments, the mesenchymal stem cell secretome is administered to a particular targeted area and is formulated so as not to spread to other surrounding areas.

In some embodiments, the mesenchymal stem cell secretome is administered to a particular targeted area and is formulated to stay in the targeted area for at least 1 minute, at least about 2 minutes, 3 at least about minutes, at least about 4 minutes, at least about 5 minutes, at least about 10 minutes, at least about 15 minutes, at least about 20 minutes, at least about 30 minutes, at least about 40 minutes, at least about 50 minutes, at least about 60 minutes, at least about 70 minutes, at least about 80 minutes, at least about 90 minutes, or at least about 2 hours.

In some embodiments, the mesenchymal stem cell secretome is administered to an affected area immediately after the wound or injury. In some embodiments, the mesenchymal stem cell secretome is administered to an affected area within 15 seconds, 30 seconds, 1 minutes, 2 minutes, 3 minutes, 4 minutes, 5 minutes, 10 minutes, 15 minutes, 20 minutes, 30 minutes, 40 minutes, 50 minutes, 60 minutes, 70 minutes, 80 minutes, 90 minutes, 2 hours, 4 hours, 8 hours, 12 hours, 24 hours, 36 hours, 48 hours, or 96 hours.

In some embodiments, the mesenchymal stem cell secretome is administered topically. In some embodiments, the mesenchymal stem cell secretome is administered by subconjunctival injection. In some embodiments, the mesenchymal stem cell secretome is administered by intravitreal injection. In some embodiments, the MSC secretome compositions exhibit ultrapotency when administered to a subject in need thereof. In some embodiments, the mesenchymal stem cell secretome is administered topically once, two, three, four, five, and/or up to six times daily. In some embodiments, the MSC secretome compositions allow for therapeutic efficacy with one drop or one administration per day. In some embodiments, one drop is administered 1, 2, 3, 4, 5, or 6 times per day. In some embodiments, one drop is administered at 1 hour, 2 hour, 3 hour, or 4 hour intervals. In some embodiments, one drop is administered at least once per day for 1 week, 2 weeks, 3 weeks, 4 weeks, 5 weeks, 6 weeks, 7 weeks, 8 weeks, 9 weeks, or 10 weeks. In some embodiments, one drop is administered at least twice per day for 1 week, 2 weeks, 3 weeks, 4 weeks, 5 weeks, 6 weeks, 7 weeks, 8 weeks, 9 weeks, or 10 weeks. In some embodiments, one drop is administered at least 3 times per day for 1 week, 2 weeks, 3 weeks, 4 weeks, 5 weeks, 6 weeks, 7 weeks, 8 weeks, 9 weeks, or 10 weeks. In some embodiments, one drop is administered at least 4 times per day for 1 week, 2 weeks, 3 weeks, 4 weeks, 5 weeks, 6 weeks, 7 weeks, 8 weeks, 9 weeks, or 10 weeks. In some embodiments, one drop is administered at least 5 times per day for 1 week, 2 weeks, 3 weeks, 4 weeks, 5 weeks, 6 weeks, 7 weeks, 8 weeks, 9 weeks, or 10 weeks. In some embodiments, one drop is administered at least 6 times per day for 1 week, 2 weeks, 3 weeks, 4 weeks, 5 weeks, 6 weeks, 7 weeks, 8 weeks, 9 weeks, or 10 weeks.

In some embodiments of the method of treatment for an ocular condition in a subject in need thereof comprising administering to the subject a mesenchymal stem cell (MSC) secretome composition, wherein the MSC secretome composition comprises:
i. at least one trophic factors/cytokines selected from the group consisting of HGF, TIMP-1, TIMP-2, PAI-1 (Serpin E1), VEGF-A, and b-NGF;
ii. at least one additional factor selected from the group consisting of PEDF (Serpin F1), IGFBP-2, IGFBP-3, SDF-1, TSG-14, Kallikrein 3, MCP-1, Angiogenin, MCP-2, Angio-2, IL-6, IL-17, G-CSF, M-CSF, GM-CSF, IL-8, TNF-beta, and PDGF; and
iii. at least one additional factor selected from the group consisting of DPPIV (dipeptidyl peptidase-4), uPA, Angiopoietin-1, Angiopoietin-2, Amphiregulin, Endostatin, Endothelin-1, Thrombospondin-2, and Thrombospondin-1.

In some embodiments, the MSC secretome composition for use in the methods of treatment further comprises high levels of at least one factor selected from the group consisting of Serpin E1, Serpin A1, TIMP-1, Thrombospondin-1, Pentraxin-3 (TSG-14), Platelet Factor 4, and Serpin F1.

In some embodiments, the MSC secretome composition for use in the methods of treatment comprises 1 ng/mL—100 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition for use in the methods of treatment comprises 1 ng/mL-200 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition for use in the methods of treatment comprises 1 ng/mL-300 ng/mL of at least one factor selected from the group consisting of Serpin E1, Serpin A1, TIMP-1, Thrombospondin-1, Pentraxin-3 (TSG-14), Platelet Factor 4, and Serpin F1. In some embodiments, the MSC secretome composition for use in the methods of treatment comprises 1 ng/mL-400 ng/mL of at least one factor selected from the group consisting of Serpin E1, Serpin A1, TIMP-1, Thrombospondin-1, Pentraxin-3 (TSG-14), Platelet Factor 4, and Serpin F1.

In some embodiments, the MSC secretome composition for use in the methods of treatment further comprises mid-range levels of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the MSC secretome composition for use in the methods of treatment comprises 400 pg/mL-3000 pg/mL of at least one factor selected from the group consisting of Angiopoietin-1, Angiopoietin-2, Amphiregulin, Endostatin, Endothelin-1, Thrombospondin-2, Thrombospondin-1, Angiogenin, DPPIV, IGFBP-3, and uPA.

In some embodiments, the MSC secretome composition for use in the methods of treatment further comprises at least one factor selected from the group consisting of Apolipoprotein A1, Complement Factor D, Complement factor H, Complement factor I, C1 esterase inhibitor (C1-INH), C4b-binding protein (C4BP), CD46, Complement receptor type 1 (CR1), C-reactive protein, Cystatin C, DKK-1, Emmprin, Osteopontin, vitamin D BP, MIF, RANTES, uPAR, IL-17a, GDF-15, and IFNγ.

In some embodiments, the MSC secretome composition for use in the methods of treatment comprises ratios of anti-angiogenic to pro-angiogenic wherein the ratio is >2, >3, >4, or >5. In some embodiments, the anti-angiogenic factors includes one or more factors selected from the group consisting of PEDF, lower levels of VEGF, and Serpin E1 and pro-angiogenic: VEGF, Angiogenin, IGFBP-3, uPA, Angio-1, Angio-2, Endothelin-1.

In some embodiments, the MSC secretome composition for use in the methods of treatment further comprises low levels for VEGF. In some embodiments, the MSC secretome for use in the methods of treatment comprises 1 pg/mL-400 pg/mL of VEGF. In some embodiments, the level of VEGF is 5-10 fold lower than the level of Serpin E1. In some embodiments, the MSC secretome composition for use in the methods of treatment comprises one or more anti-angiogenic factor, and wherein the sum of the concentration of the one or more anti-angiogenic factors relative to the concentration of VEGF is >2, >3, >4, or >5.

In some embodiments, the MSC secretome composition for use in the methods of treatment does not comprise or comprises very low levels of bFGF, PLGF, and PDGF.

In some embodiments, the MSC secretome composition for use in the methods of treatment comprises less than 1000 pg/mL of bFGF, PLGF, and PDGF.

In some embodiments, the MSC secretome composition for use in the methods of treatment has a pH of about 4.7 to about 7.5.

In some embodiments, the MSC secretome composition for use in the methods of treatment is formulated in a buffer system selected from the group consisting of di/mono sodium phosphate, sodium citrate/citric acid, boric acid/sodium citrate, boric acid/sodium tetraborate, and citric acid/disodium phosphate.

In some embodiments, the MSC secretome composition for use in the methods of treatment further comprises a tonicity modifying agent. In some embodiments, the tonicity modifying agent is selected from the group consisting of NaCl, KCl, mannitol, dextrose, sucrose, sorbitol, and glycerin.

In some embodiments, the MSC secretome composition for use in the methods of treatment further comprises mono/di-sodium phosphate, mannitol, and trehalose, and wherein the composition has a pH of about pH 7.4.

In some embodiments, the MSC secretome composition for use in the methods of treatment further comprises divalent cations. In some embodiments, the divalent cations are selected from the group consisting of Mg2+, Ca2+, and Zn2+.

In some embodiments, the MSC secretome composition for use in the methods of treatment further comprises di-sodium phosphate/citric acid, mannitol, and trehalose, and wherein the composition has a pH of about pH 6.4.

In some embodiments, the MSC secretome composition for use in the methods of treatment further comprises an adhesive agent. In some embodiments, the adhesive agent is selected from the group consisting of hypromellose, Poloxamer 407, Poloxamer 188, Poloxomer 237, Poloxomer 338, Hypromellose, (HPMC), polycarbophil, polyvinylpyrrolidone (PVP), Polyvinyl alcohol (PVA), polyimide, sodium hyaluronate, gellan gum, poly(lactic acid-co-glycolic acid) (PLGA), polysiloxane, polyimide, carboxymethylcellulose (CMC), or hydroxypropyl methylcellulose (HPMC), hydroxy methyl cellulose, hydroxy ethyl cellulose, sodium carboxy methyl cellulose, fibrin glue, polyethyelene glycol, and GelCORE.

In some embodiments, the MSC secretome composition for use in the methods of treatment does not comprise one or more components selected from the group consisting of: xenobiotic components; Phenol red; peptides and biomolecules <3 kDa; antibiotics; protein aggregates >200 nm; cells; non-exosome/non-Extracellular Vesicles cell debris; hormones; and L-glutamine.

In some embodiments, the MSC secretome composition for use in the methods of treatment comprises: HGF; Pentraxin-3 (TSG-14); VEGF; TIMP-1; Serpin E1; and <5 ng/mL IL-8.

In some embodiments, the MSC secretome for use in the methods of treatment composition comprises:
i. 0.3-4.5 ng/mL HGF;
ii. 0.5-20 ng/mL Pentraxin-3 (TSG-14);
iii. 100-600 pg/mL VEGF;
iv. 10-200 ng/mL TIMP-1;
v. 20-80 ng/mL Serpin E1; and
vi. <5 ng/mL IL-8.

In some embodiments, the MSC secretome composition for use in the methods of treatment comprise an anti-angiogenic MSC secretome or an anti-scarring MSC secretome.

In some embodiments, the present disclosure provides a method of treatment for an ocular condition in a subject in need thereof comprising administering to the subject a mesenchymal stem cell (MSC) secretome composition, wherein the MSC secretome composition is a stable mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg hypromellose per mL; and
wherein the pH is about 4.7 to about 7.5.

In some embodiments, the present disclosure provides a method of treatment for an ocular condition in a subject in need thereof comprising administering to the subject a mesenchymal stem cell (MSC) secretome composition, wherein the MSC secretome composition is a stable mesenchymal stem cell (MSC) secretome formulation comprising:
i. 0.004%-0.08% w/w of MSC secretome
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w hypromellose; and
wherein the pH is about 4.7 to about 7.5.

In some embodiments, the present disclosure provides a method of treatment for an ocular condition in a subject in need thereof comprising administering to the subject a mesenchymal stem cell (MSC) secretome composition, wherein the MSC secretome composition is a stable mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate;
vi. 0.5 mg-2 mg optionally hypromellose per mL; and
wherein the pH is about 4.7 to about 7.5.

In some embodiments, the present disclosure provides a method of treatment for an ocular condition in a subject in need thereof comprising administering to the subject a mesenchymal stem cell (MSC) secretome composition, wherein the MSC secretome composition is a stable mesenchymal stem cell (MSC) secretome formulation comprising:
i. 0.004%-0.08% w/w of MSC secretome
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate;
vi. 1%-3% w/w optionally hypromellose; and
wherein the pH is about 4.7 to about 7.5.

In some embodiments, the present disclosure provides a method of treatment for an ocular condition in a subject in need thereof comprising administering to the subject a mesenchymal stem cell (MSC) secretome composition, wherein the MSC secretome composition is a stable mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate; and
wherein the pH is about 4.7 to about 7.5.

In some embodiments, the present disclosure provides a method of treatment for an ocular condition in a subject in need thereof comprising administering to the subject a mesenchymal stem cell (MSC) secretome composition, wherein the MSC secretome composition is a stable mesenchymal stem cell (MSC) secretome formulation comprising:
i. 0.004%-0.08% w/w of MSC secretome
ii. 4%-5% w/w monobasic sodium phosphate;
iii. 21.5%-23% w/w dibasic sodium phosphate;
iv. 23%-25% w/w mannitol;
v. 46%-48% w/w trehalose dehydrate; and
wherein the pH is about 4.7 to about 7.5.

### F. Kit

A kit can include an MSC secretome in a container or the conditioned media for use in preparing an MSC secretome, also in a container, as disclosed herein, and instructions for use. Additionally, a kit can include components for mixing to prepare a solution for use in an ocular treatment, and instructions for mixing and use.

The container can include at least one vial, well, test tube, flask, bottle, syringe, or other container means, into which an MSC secretome in a container or the conditioned media for use in preparing an MSC secretome, and in some instances, suitably aliquoted. Where an additional component is provided, the kit can contain additional containers into which this component may be placed. Such containers may include injection or blow-molded plastic containers into which the desired vials are retained. Containers and/or kits can include labeling with instructions for use and/or warnings.

The present disclosure is further illustrated by the following examples, which should not be construed as further limiting. The contents of all figures and all references, Genbank sequences, patents and published patent applications cited throughout this application are expressly incorporated herein by reference.

The present invention can provide kits comprising a panel of tests and/or assays for characterizing a MSC secretome, wherein the panel comprises at least two characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, tissue explant survival and function assays, organoid development or survival/function assays, in vivo response to oxidative stress (for example, retinal ischemia reperfusion), epithelial barrier integrity assays, retinal degeneration assays, and/or assays of inherited retinal disease including human and animal retinal explants, neural protection/neurotrophic assays. In some embodiments, the panel of tests and/or assays identifies a MSC secretome as described herein.

The present invention can provide kits comprising a panel of tests and/or assays for determining consistency between MSC secretome lots, wherein the panel comprises one or more characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, tissue explant survival and function assays, organoid development or survival/function assays, epithelial barrier integrity assays, retinal degeneration assays, and/or assays of inherited retinal disease including human and animal retinal explants, neural protection/neurotrophic assays. In some embodiments, the panel of tests and/or assays identifies a MSC secretome as described herein.

### EXAMPLES

### Example 1: Cellular Response to Secretome

A. Evaluation of Oxidative Stress and Cell Survival

Stressors: 7-ketocholesterol (7-KC), sodium periodate (NaIO₄), hydrogen peroxide (H₂O₂), all-trans retinoic acid (ATRA), or tert-butyl hydroperoxide (t-BHP). Positive controls: N-acetylcysteine (NAC). Cells are selected from ARPE-19, primary human RPE, MIO-M1 human Müller cells, or 661 W cell lines.

General Procedure: in 96 well plates seed cells at 30% confluency and allow to adhere overnight. Cells are then loaded with chloromethyl derivative of H₂DCFDA (CM-H₂DCFDA) and MitoSOX Red for 30 minutes, washed, and treated with varying concentrations of 7-KC, NaIO₄, H₂O₂, ATRA, or t-BHP in the absence/presence of NAC for 1 hour. Cells are then washed and relative levels of ROS production are measured. In parallel, cell viability by MTT measurement is monitored 24 hours post treatment for the same experimental conditions.

Dose-dependent elevation in ROS is evaluated as indicated by CM-H₂DCFDA and MitoSOX Red, then correlated with cell survival at 24 hrs post insult. All data is normalized to the NAC positive control.

B. Evaluation of Oxidative Stress and Secretome Benefit

Based on the results and findings described under part A, two to three oxidative stress conditions will be selected to test secretome activity. The positive control used is NAC. Initial studies are run in parallel to monitor ROS levels achieved in the study. The selected endpoint is cell survival by Microtiter Tetrazolium (MTT) assay.

After exposure to selected oxidative insult, cells are treated with various concentrations of secretome, and cell viability is evaluated at 24 hours post insult by MTT. The negative controls are basal medium with 1% serum, as well as heat-denatured secretome. The positive control is NAC.

Prior to exposure to selected oxidative insult, cells are treated for 24 hours with various concentrations of secretome, then challenged with the selected oxidative insult; ROS levels are evaluated using CM-H₂DCFDA and MitoSOX Red, in parallel to cell viability as measured by MTT. The negative controls consist of a medium with 1% serum condition, and a heat-denatured secretome condition, as the positive control consists of NAC.

The impact of secretome on cellular ROS production and cell viability is evaluated under protective conditions.

In some embodiments, the secretome is added before the cells are treated with the oxidative stress inducers. In some embodiments, the secretome is added during the cells are treated with the oxidative stress inducers. In some embodiments, the secretome is added after the cells are treated with the oxidative stress inducers.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method for characterizing a MSC secretome, or for determining biopotency and stability of a MSC secretome, or for determining MSC secretome lot consistency between a plurality of MSC secretome lots, wherein the method comprises:
(i) subjecting an MSC secretome to one or more characterization assays, wherein characterization assays are selected from the group consisting of physical component characterizations, oxidative stress assays, misfolded protein response assays, ER stress assays, cellular response assays, safety analyses, stability assays, proliferation assays, migration assays, adhesion assays, neovascularization assays, differentiation/scarring assays, inflammation assays, tissue explant survival and function assays, organoid development or survival/function assays, epithelial barrier integrity assays, retinal degeneration assays, and/or assays of inherited retinal disease including human and animal retinal explants, neural protection/neurotrophic assays, wherein the MSC secretome is optionally preconditioned; and
(ii) determining the results from the one or more assays in (i).

2.-13. (canceled)

14. A panel of tests and/or assays for characterizing a MSC secretome, or for determining biopotency and stability of a MSC secretome, or for determining consistency between MSC secretome lots, wherein the panel comprises one or more characterization assays, wherein characterization assays are selected from the group consisting of physical component characterizations, oxidative stress assays, misfolded protein response assays, ER stress assays, safety analyses, stability assays, proliferation assays, migration assays, adhesion assays, neovascularization assays, differentiation/scarring assays, inflammation assays, epithelial barrier integrity assays, retinal degeneration assays, and/or assays of inherited retinal disease including human and animal retinal explants, neural protection/neurotrophic assays, wherein the secretome is optionally preconditioned.

15.-60. (canceled)

61. A bone marrow-derived mesenchymal stem cell (MSC) secretome composition comprising: HGF; Pentraxin-3 (TSG-14); VEGF; TIMP-1; Serpin E1; <5 ng/mL IL-8, and a tonicity modifying agent, wherein the MSC secretome is preconditioned.

62.-82. (canceled)

83. A stable bone marrow-derived mesenchymal stem cell (MSC) secretome formulation comprising:
i. 2 μg-20 μg of MSC secretome per mL;
ii. 2 mg-3 mg monobasic sodium phosphate per mL;
iii. 11 mg-12 mg dibasic sodium phosphate per mL;
iv. 11.5 mg-13 mg mannitol per mL;
v. 23 mg-24 mg trehalose dihydrate; and
vi. 0.5 mg-2 mg hypromellose per mL;
and wherein the pH is about 4.7 to about 7.5, wherein the MSC secretome is preconditioned.

84. A stable bone marrow-derived mesenchymal stem cell (MSC) secretome formulation comprising:
i. 0.004%-0.08% w/w of MSC secretome;
ii. 4%-5% w/w monobasic sodium phosphate;
iii. 21.5%-23% w/w dibasic sodium phosphate; iv. 23%-25% w/w mannitol;
v. 46%-48% w/w trehalose dehydrate; and
vi. 1%-3% w/w hypromellose; and
wherein the pH is about 4.7 to about 7.5, wherein the MSC secretome is preconditioned.

85. A stable bone marrow-derived mesenchymal stem cell (MSC) secretome formulation comprising:
i. sodium phosphate 10 mM;
ii. histidine HCL 10 mM;
iii. trehalose dihydrate 10%; and
iv. polysorbate 20 0.01%;
and wherein the pH is about 5.5, wherein the MSC secretome is preconditioned.

86. A stable bone marrow-derived mesenchymal stem cell (MSC) secretome formulation comprising:
i. sodium phosphate 10 mM;
ii. sucrose 5%; and
iii. sodium chloride 10 mM;
and wherein the pH is about 6.2, wherein the MSC secretome is preconditioned.

87. A stable bone marrow-derived mesenchymal stem cell (MSC) secretome formulation comprising:
i. sodium phosphate 10 mM;
ii. sucrose 5.8%; and
iii. polysorbate 80 0.02%;
and wherein the pH is about 6.2, wherein the MSC secretome is preconditioned.

88. A method of treatment for an ocular condition in a subject in need thereof comprising administering to the subject a bone marrow-derived mesenchymal stem cell (MSC) secretome composition, wherein the MSC secretome composition comprises: HGF; Pentraxin-3 (TSG-14); VEGF; TIMP-1; Serpin E1; and <5 ng/mL IL-8, wherein the MSC secretome is preconditioned.

89.-134. (canceled)

135. A composition that induces ocular wound healing comprising a mesenchymal stem cell (MSC) secretome and a tonicity modifying agent, wherein the MSC secretome is preconditioned, wherein the ability of the composition to promote ocular wound healing is indicated by a wound healing assay comprising:
a) providing a layer of corneal cells;
b) introducing a wound gap to the layer of corneal cells; and
c) determining whether the wound gap heals in the presence of the composition,
wherein the composition is administered to the corneal cells either before or after step b);
wherein closure of the wound gap is indicative of the ability of the composition to induce ocular wound healing.

136.-153. (canceled)

154. A method of preconditioning a MSC secretome, comprising affecting secretory profile for the MSCs comprising one or more of the following: change in culture format (e.g., 2D planar vs. 3D bioreactor), different biomaterial scaffolds, co-culture, addition of pharmacological compounds, growth factors, chemokines, addition of toll-like receptor agonists, inflammatory cytokines, advanced glycation end products (AGEs), oxidized phospholipids, Malondialdehyde, or carboxyethylpyrrole, agitation presence of ECM, culture under sheer stress, agitation or suspension as aggregate or within a matrix, induced misfolded protein response, ER stress, induction of differentiation of the MSCs, culture in the presence of conditioned media and hypoxia/anoxia.

155.-167. (canceled)
